(19) United States
(12) Patent Application Publication
(10) Pub. No.: US 2026/0009018 A1
(43) Pub. Date: Jan. 8, 2026

(54) REVERSE TRANSCRIPTASE FOR NUCLEIC ACID SEQUENCING

(71) Applicant: Element Biosciences, Inc., San Diego, CA (US)

(72) Inventors: Matthew KELLINGER, San Diego, CA (US); Molly HE, San Diego, CA (US); Tyler LOPEZ, San Diego, CA (US)

(21) Appl. No.: 19/066,925

(22) Filed: Feb. 28, 2025

Related U.S. Application Data
(63) Continuation of application No. 18/766,232, filed on Jul. 8, 2024, now abandoned.
Application No. 18/766,232 is a continuation of application No. 17/120,030, filed on Dec. 11, 2020, now Pat. No. 12,091,657.
Application No. 17/120,030 is a continuation of application No. PCT/US2019/036631, filed on Jun. 11, 2019.
(60) Provisional application No. 62/684,115, filed on Jun. 12, 2018.

(51) Int. Cl. C12N 15/10; C12N 9/12; C12P 21/00; C12Q 1/6874

(52) U.S. Cl. CPC C12N 15/102; C12N 9/1276; C12N 15/1068; C12Q 1/6874; C12Y 207/07049; C12N 2310/321; C12P 21/00

(57) ABSTRACT

Provided herein are compositions and methods for the incorporation of unnatural nucleotides using mutant polymerases, such as reverse transcriptases. Further provided herein are methods of detection and sequencing of polynucleotide sequences. In some aspects, the compositions and methods are used enhance the efficiency and speed of detecting nucleotide bases. The methods and compositions described herein may further reduce time, cost, or scale of devices for next generation sequencing platforms.

### CROSS-REFERENCE

This application is a continuation application of U.S. application Ser. No. 18/766,232, filed Jul. 8, 2024, which is a continuation application of U.S. application Ser. No. 17/120,030, filed Dec. 11, 2020, now U.S. Pat. No. 12,091,657, which is a continuation application of International Application No. PCT/US2019/036631, filed Jun. 11, 2019, which claims the benefit of U.S. Provisional Application No. 62/684,115, filed Jun. 12, 2018, which is incorporated herein by reference in its entirety.

### SEQUENCE LISTING

The instant application contains a Sequence Listing which has been submitted electronically in XML format and is hereby incorporated by reference in its entirety. Said XML copy, created Aug. 26, 2025, is named ELEM-045_C03US_sequence_listing.xml and is 408,990 bytes in size.

### BACKGROUND OF THE INVENTION

Next-generation sequencing (NGS) techniques have become a powerful tool for acquiring sequencing data used in molecular biology techniques, taxonomy, agriscience, medical diagnostics, and the development of new therapies. For example, sequencing-by-synthesis (SBS) methods are used to extend a growing polynucleotide chain with a polymerase, while analyzing the identity of matching complementary nucleotides that are incorporated. However, additional methods to increase the sensitivity, accuracy, scalability, and cost efficiency of these methods is needed.

### SUMMARY OF THE INVENTION

Provided herein are methods, compositions, and systems that facilitate the addition of 3′ modified nucleotides into a nucleotide chain. Provided herein are polypeptides comprising mutations that differ from wild-type polypeptides, such as at positions in a retrotranscriptase-like domain. Provided herein are polypeptides having at least 85% identity to SEQ ID NO: 2, and differing from SEQ ID NO: 2 at at least one of positions K152, D153, A154, F155, F156, and Q190 relative to SEQ ID NO: 1. Also provided herein are polypeptides wherein the polypeptide has at least 90% identity to SEQ ID NO: 2. Also provided herein are polypeptides wherein the polypeptide has at least 95% identity to SEQ ID NO: 2. Also provided herein are polypeptides wherein the polypeptide comprises a mutation at at least two of positions K152, D153, A154, F155, F156, and Q190. Also provided herein are polypeptides wherein the polypeptide comprises a mutation at at least three of positions K152, D153, A154, F155, F156, and Q190. Also provided herein are polypeptides wherein the polypeptide comprises a mutation at at least four of positions K152, D153, A154, F155, F156, and Q190. Also provided herein are polypeptides wherein the polypeptide comprises a mutation at at least five of positions K152, D153, A154, F155, F156, and Q190. Also provided herein are polypeptides wherein the polypeptide comprises a mutation at position K152. Also provided herein are polypeptides wherein the polypeptide comprises a substitution at K152. Also provided herein are polypeptides wherein the mutation is K152R. Also provided herein are polypeptides wherein the mutation is K152C. Also provided herein are polypeptides wherein the mutation is K152T. Also provided herein are polypeptides wherein the mutation is K152L. Also provided herein are polypeptides wherein the polypeptide comprises a mutation at position D153. Also provided herein are polypeptides wherein the polypeptide comprises a substitution at D153. Also provided herein are polypeptides wherein the mutation is D153Y. Also provided herein are polypeptides wherein the polypeptide comprises a mutation at position A154. Also provided herein are polypeptides wherein the polypeptide comprises a substitution at A154. Also provided herein are polypeptides wherein the mutation is A154Y. Also provided herein are polypeptides wherein the polypeptide comprises a mutation at position F155. Also provided herein are polypeptides wherein the polypeptide comprises a substitution at F155. Also provided herein are polypeptides wherein the mutation is F155A. Also provided herein are polypeptides wherein the polypeptide comprises a mutation at position F156. Also provided herein are polypeptides wherein the polypeptide comprises a substitution at F156. Also provided herein are polypeptides wherein the mutation is F156N. Also provided herein are polypeptides wherein the polypeptide comprises a mutation at position Q190. Also provided herein are polypeptides wherein the polypeptide comprises a substitution at Q190. Also provided herein are polypeptides wherein the mutation is Q190A or Q190S.

Provided herein are mutant nucleic acid polymerases, wherein the nucleic acid polymerase exhibits increased incorporation of 3′ methylazido nucleotide relative to a nearest related wild-type sequence. Also provided herein are mutant nucleic acid polymerases wherein

the nucleic acid polymerase sequence differs from a nearest related wild-type sequence by at least one position in an RT domain. Also provided herein are mutant nucleic acid polymerases wherein the nucleic acid polymerase exhibits at least 30% higher incorporation of 3′ methylazido-dUTP relative to a nearest wild type sequence under conditions comprising contacting a polynucleotide template with the nucleic acid polymerase and 10 uM 3′ methylazido-dUTP for 20 minutes. Also provided herein are mutant nucleic acid polymerases wherein the nucleic acid polymerase exhibits at least 20% higher incorporation of 3′ methylazido-dUTP relative to a nearest wild type sequence under conditions comprising contacting a polynucleotide template with the nucleic acid polymerase and 10 uM 3′ methylazido-dUTP for 20 minutes. Also provided herein are nucleic acid polymerases wherein the nucleic acid polymerase is a reverse transcriptase family polymerase, or comprises a reverse transcriptase domain. Also provided herein are nucleic acid polymerases wherein the nucleic acid polymerase comprises a sequence that has at least 85% identity to SEQ ID NO: 2. Also provided herein are nucleic acid polymerases wherein the nucleic acid polymerase comprises a sequence that has at least 90% identity to SEQ ID NO: 2.

Provided herein are nucleic acid polymerases exhibiting increased kcat for incorporation of 3′ methylazido-dUTP relative to a nearest related wild-type sequence.

Provided herein are nucleic acid polymerases exhibiting decreased Km of 3′ methylazido-dUTP relative to a nearest related wild-type sequence.

Provided herein are nucleic acid polymerases exhibiting increased kcat/Km of 3′ methylazido-dUTP relative to a nearest related wild-type sequence.

Provided herein are polypeptides comprising a fragment comprising the sequence of SEQ ID NO: 4, wherein the fragment further comprises an adjacent N-terminal aspartic acid, adjacent C-terminal arginine, or a combination thereof.

Provided herein are polypeptides comprising a fragment comprising the sequence of SEQ ID NO: 5, wherein the fragment further comprises an adjacent N-terminal glutamine, adjacent C-terminal histidine, or a combination thereof.

Provided herein are polypeptides comprising a fragment comprising the sequence of SEQ ID NO: 6, wherein the fragment further comprises an adjacent N-terminal valine, adjacent C-terminal cysteine, or a combination thereof.

Provided herein are polypeptides comprising a fragment comprising the sequence of SEQ ID NO: 7, wherein the fragment further comprises an adjacent N-terminal threonine, adjacent C-terminal cysteine, or a combination thereof.

Provided herein are polypeptides comprising a fragment comprising the sequence of SEQ ID NO: 8, wherein the fragment further comprises an adjacent N-terminal threonine, adjacent C-terminal cysteine, or a combination thereof.

Provided herein are polypeptides comprising a fragment comprising the sequence of SEQ ID NO: 9, wherein the fragment further comprises an adjacent N-terminal aspartic acid, adjacent C-terminal leucine, or a combination thereof.

Provided herein are polypeptides comprising a fragment comprising the sequence of SEQ ID NO: 10, wherein the fragment further comprises an adjacent N-terminal aspartic acid, adjacent C-terminal arginine, or a combination thereof.

Provided herein are polypeptides comprising a fragment comprising the sequence of SEQ ID NO: 11, wherein the fragment further comprises an adjacent N-terminal threonine, adjacent C-terminal threonine, or a combination thereof.

Provided herein are polypeptides comprising a fragment comprising the sequence of SEQ ID NO: 12, wherein the fragment further comprises an adjacent N-terminal threonine, adjacent C-terminal asparagine, or a combination thereof.

Provided herein are polypeptides comprising a fragment comprising the sequence of SEQ ID NO: 13, wherein the fragment further comprises an adjacent N-terminal threonine, adjacent C-terminal asparagine, or a combination thereof.

Provided herein are polypeptides comprising a fragment comprising the sequence of SEQ ID NO: 14, wherein the fragment further comprises an adjacent N-terminal threonine, adjacent C-terminal serine, or a combination thereof.

Provided herein are polypeptides comprising a reverse transcriptase domain, wherein the polypeptide comprises at least one mutation in an RNA/DNA binding site relative to a nearest related wild-type sequence. Also provided herein are polypeptides wherein the polypeptide comprises a sequence with at least 85% identity to any one of SEQ ID NOs: 1-4, 17, or 18. Also provided herein are polypeptides wherein the polypeptide comprises a sequence with at least 90% identity to any one of SEQ ID NOs: 1-4, 17, or 18.

Provided herein are polypeptides comprising a reverse transcriptase domain, wherein the polypeptide further comprises at least one mutation in a nucleotide binding site relative to a nearest related wild-type sequence. Also provided herein are polypeptides wherein the polypeptide comprises a sequence with at least 85% identity to any one of SEQ ID NOs: 1-4, 17, or 18. Also provided herein are polypeptides wherein the polypeptide comprises a sequence with at least 90% identity to any one of SEQ ID NOs: 1-4, 17, or 18.

Provided herein are polypeptides comprising a reverse transcriptase domain, wherein the polypeptide further comprises at least one of K152C, K152T, K152L, D153Y, A154Y, F155A, F156N, and Q190A, or Q190S as identified with reference to the residues of FIG. 4. Also provided herein are polypeptides wherein the polypeptide comprises a sequence with at least 85% identity to any one of SEQ ID NOs: 1-4, 17, or 18. Also provided herein are polypeptides wherein the polypeptide comprises a sequence with at least 90% identity to any one of SEQ ID NOs: 1-4, 17, or 18.

Provided herein are methods of incorporating a nucleotide analogue into a polynucleotide primer comprising contacting the nucleotide analogue to a polymerase described herein. Also provided herein are methods wherein the nucleotide analogue comprises a 3′ OH blocking group. Also provided herein are methods wherein the 3′ OH blocking group comprises at least one of an azido, aminoxy, nitro, and a disulfide radical. Also provided herein are methods wherein the nucleotide analogue comprises a modified base. Also provided herein are methods wherein the modified base comprises a detectable moiety. Also provided herein are methods wherein the detectable moiety comprises a fluorescent label.

Provided herein are methods of polynucleotide extension, comprising incorporating a nucleotide analogue into a polynucleotide primer using a nucleic acid polymerase, wherein the nucleic acid polymerase exhibits an increased incorporation of 3′ methylazido-dUTP relative to a nearest related wild-type sequence. Also provided herein are methods wherein the nucleic acid polymerase is a reverse transcriptase. Also provided herein are methods wherein the nucleic acid polymerase exhibits at least 10% higher incorporation of 3′ methylazido-dUTP relative to a nearest related wild-type sequence under conditions comprising contacting the nucleic acid polymerase with a polynucleotide primer and 10 uM 3′ methylazido-dUTP for 20 minutes. Also provided herein are methods wherein the nucleic acid polymerase exhibits at least 30% higher incorporation of 3′ methylazido-dUTP relative to a nearest related wild-type sequence under conditions comprising contacting the nucleic acid polymerase with a polynucleotide primer and 10 uM 3′ methylazido-dUTP for 20 minutes. Also provided herein are methods wherein the nucleic acid polymerase exhibits at least 50% higher incorporation of 3′ methylazido-dUTP relative to a nearest related wild-type sequence under conditions comprising contacting the nucleic acid polymerase with a polynucleotide primer and 10 uM 3′ methylazido-dUTP for 20 minutes. Also provided herein are methods wherein the nucleic acid polymerase exhibits at least 30% higher incorporation of 3′ methylazido-dUTP relative to SEQ ID NO: 2 under conditions comprising contacting the nucleic acid polymerase with a polynucleotide primer and 10 uM 3′ methylazido-dUTP for 20 minutes.

Provided herein are methods of polynucleotide extension, comprising incorporating a nucleotide analogue into a polynucleotide primer using a nucleic acid polymerase, wherein the nucleic acid polymerase exhibits a decreased Km for 3′ methylazido-dUTP relative to the nearest related wild-type sequence. Also provided herein are methods wherein the nucleic acid polymerase having at least 90% identity to SEQ ID NO:2 and a mutation at at least one of positions K152, D153, A154, F155, F156, and Q190. Also provided herein are methods wherein the polypeptide comprises a mutation at at least two of positions K152, D153, A154, F155, F156, and Q190. Also provided herein are methods wherein the polypeptide comprises a mutation at at least three of positions K152, D153, A154, F155, F156, and Q190. Also provided herein are methods wherein the polypeptide comprises a mutation at at least four of positions K152, D153, A154, F155, F156, and Q190. Also provided herein are methods wherein the polypeptide comprises a mutation at at least five of positions K152, D153, A154, F155, F156, and Q190. Also provided herein are methods wherein the polypeptide comprises a mutation at position K152. Also provided herein are methods wherein the polypeptide comprises a substitution at K152. Also provided herein are methods wherein the mutation is K152R. Also provided herein are methods wherein the mutation is K152C. Also provided herein are methods wherein the mutation is K152T. Also provided herein are methods wherein the mutation is K152L. Also provided herein are methods wherein the polypeptide comprises a mutation at position D153. Also provided herein are methods wherein the polypeptide comprises a substitution at D153. Also provided herein are methods wherein the mutation is D153Y. Also provided herein are methods wherein the polypeptide comprises a mutation at position A154. Also provided herein are methods wherein the polypeptide comprises a substitution at A154. Also provided herein are methods wherein the mutation is A154Y. Also provided herein are methods wherein the polypeptide comprises a mutation at position F155. Also provided herein are methods wherein the polypeptide comprises a substitution at F155. Also provided herein are methods wherein the mutation is F155A. Also provided herein are methods wherein the polypeptide comprises a mutation at position F156. Also provided herein are methods wherein the polypeptide comprises a substitution at F156. Also provided herein are methods wherein the mutation is F156N. Also provided herein are methods wherein the polypeptide comprises a mutation at position Q190. Also provided herein are methods wherein the polypeptide comprises a substitution at Q190. Also provided herein are methods wherein the mutation is Q190A or Q190S. Also provided herein are methods wherein incorporating further comprises contact with a polynucleotide template.

Provided herein are methods of polynucleotide extension, comprising incorporating a nucleotide analogue into a polynucleotide primer using a nucleic acid polymerase, wherein the nucleic acid polymerase exhibits an increased kcat for incorporation of 3′ methylazido-dfUTP relative to the nearest related wild-type sequence. Also provided herein are methods wherein the nucleic acid polymerase having at least 90% identity to SEQ ID NO:2 and a mutation at at least one of positions K152, D153, A154, F155, F156, and Q190. Also provided herein are methods wherein the polypeptide comprises a mutation at at least two of positions K152, D153, A154, F155, F156, and Q190. Also provided herein are methods wherein the polypeptide comprises a mutation at at least three of positions K152, D153, A154, F155, F156, and Q190. Also provided herein are methods wherein the polypeptide comprises a mutation at at least four of positions K152, D153, A154, F155, F156, and Q190. Also provided herein are methods wherein the polypeptide comprises a mutation at at least five of positions K152, D153, A154, F155, F156, and Q190. Also provided herein are methods wherein the polypeptide comprises a mutation at position K152. Also provided herein are methods wherein the polypeptide comprises a substitution at K152. Also provided herein are methods wherein the mutation is K152R. Also provided herein are methods wherein the mutation is K152C. Also provided herein are methods wherein the mutation is K152T. Also provided herein are methods wherein the mutation is K152L. Also provided herein are methods wherein the polypeptide comprises a mutation at position D153. Also provided herein are methods wherein the polypeptide comprises a substitution at D153. Also provided herein are methods wherein the mutation is D153Y. Also provided herein are methods wherein the polypeptide comprises a mutation at position A154. Also provided herein are methods wherein the polypeptide comprises a substitution at A154. Also provided herein are methods wherein the mutation is A154Y. Also provided herein are methods wherein the polypeptide comprises a mutation at position F155. Also provided herein are methods wherein the polypeptide comprises a substitution at F155. Also provided herein are methods wherein the mutation is F155A. Also provided herein are methods wherein the polypeptide comprises a mutation at position F156. Also provided herein are methods wherein the polypeptide comprises a substitution at F156. Also provided herein are methods wherein the mutation is F156N. Also provided herein are methods wherein the polypeptide comprises a mutation at position Q190. Also provided herein are methods wherein the polypeptide comprises a substitution at Q190. Also provided herein are methods wherein the mutation is Q190A or Q190S. Also provided herein are methods wherein incorporating further comprises contact with a polynucleotide template.

Provided herein are methods of polynucleotide extension, comprising incorporating a nucleotide analogue into a polynucleotide primer using a nucleic acid polymerase, wherein the nucleic acid polymerase exhibits an increased kcat/Km for incorporation of 3′ methylazido-dfUTP relative to the nearest related wild-type sequence. Also provided herein are methods wherein the nucleic acid polymerase having at least 90% identity to SEQ ID NO:2 and a mutation at at least one of positions K152, D153, A154, F155, F156, and Q190. Also provided herein are methods wherein the polypeptide comprises a mutation at at least two of positions K152, D153, A154, F155, F156, and Q190. Also provided herein are methods wherein the polypeptide comprises a mutation at at least three of positions K152, D153, A154, F155, F156, and Q190. Also provided herein are methods wherein the polypeptide comprises a mutation at at least four of positions K152, D153, A154, F155, F156, and Q190. Also provided herein are methods wherein the polypeptide comprises a mutation at at least five of positions K152, D153, A154, F155, F156, and Q190. Also provided herein are methods wherein the polypeptide comprises a mutation at position K152. Also provided herein are methods wherein the polypeptide comprises a substitution at K152. Also provided herein are methods wherein the mutation is K152R. Also provided herein are methods wherein the mutation is K152C. Also provided herein are methods wherein the mutation is K152T. Also provided herein are methods wherein the mutation is K152L. Also provided herein are methods wherein the polypeptide comprises a mutation at position D153. Also provided herein are methods wherein the polypeptide comprises a substitution at D153. Also provided herein are methods wherein the mutation is D153Y. Also provided herein are methods wherein the polypeptide comprises a mutation at position A154. Also provided herein are methods wherein the polypeptide comprises a substitution at A154. Also provided herein are methods wherein the mutation is A154Y. Also provided herein are methods wherein the polypeptide comprises a mutation at position F155. Also provided herein are methods wherein the polypeptide comprises a substitution at F155. Also provided herein are methods wherein the mutation is F155A. Also provided herein are methods wherein the polypeptide comprises a mutation at position F156. Also provided herein are methods wherein the polypeptide comprises a substitution at F156. Also provided herein are methods wherein the mutation is F156N. Also provided herein are methods wherein the polypeptide comprises a mutation at position Q190. Also provided herein are methods wherein the polypeptide comprises a substitution at Q190. Also provided herein are methods wherein the mutation is Q190A or Q190S. Also provided herein are methods wherein incorporating further comprises contact with a polynucleotide template.

Provided herein are methods of sequencing a nucleic acid comprising: contacting the polypeptide of described herein with a nucleotide analogue, a template polynucleotide, and a polynucleotide primer; incorporating the nucleotide analogue into the polynucleotide primer; and detecting the incorporation of the nucleotide analogue.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

The invention has other advantages and features which will be more readily apparent from the following detailed description of the invention and the appended claims, when taken in conjunction with the accompanying drawings, in which:

FIG. 1 illustrates a plot of polynucleotide extension rates obtained by contacting wild-type or mutated reverse transcriptase MMLV and 3′methylazido dUTP.

FIG. 2 illustrates a plot comparing extension product formation of wild-type and mutant reverse transcriptases in the presence of 3′methylazido dUTP.

FIG. 3 illustrates an annotated MMLV sequence (SEQ ID NO: 317).

FIG. 4A illustrates an annotated sequence alignment of SEQ ID NOs: 1-4, 17, and 18 for residues 1-384 of SEQ ID NO:1.

FIG. 4B illustrates an annotated sequence alignment of SEQ ID NOs: 1-4, 17, and 18 for residues 385-672 of SEQ ID NO: 1.

FIG. 5 illustrates relative activity of further additional variants of MMLV in incorporation of 3′methylazido nucleotides at the N+1 position of an extending polynucleotide chain at 42° C. Variants are present in cleared lysates from expression strains. Leftmost bar: WT-MMLV-RT. Mutants 1-86 are listed along the X axis.

FIG. 6 shows fluorescence traces and fluorescence quantification of clarified lysates showing the activity of variants of MMLV in incorporation of 3′methylazido nucleotides at the N+1 position of an extending polynucleotide chain at 42° C. Mutations are listed in Table 4.

FIG. 7 illustrates relative activity of additional variants of MMLV in incorporation of 3′methylazido nucleotides at the N+1 position of an extending polynucleotide chain at 42° C. Variants are present in cleared lysates from expression strains. Leftmost bar: WT-MMLV-RT. Variants shown are, from left to right, Wild-Type, 1_41, 2_2, 4_40, 42_57, 1_28, 1_8, L18_3, 45_93, 1_25, 2_33, 2_28, 3_46, 1_15, 2_90, 2_34, 4_19, 2_20, 1_67, 1_48, L61_2, 3_19, 4_28, 2_72, 5_13, L05_1, 1_44, 2_46, 45_95, 42_27, 3_96, 42_2, 42_33, 42_34, L53_1, L69_1, 45_65, 5_68, L26, 4_1, 4_26, 4_44, 2_8, 45_29, L71_4, 4_10, 3_44, 4_12, 2_36, 2_29, L38_2, L64_3, 3_7, 42_25, 45_97, 3_91, 5_14, 2_41, 4_38, 4_7, 1_16, 2_55, 45_96, 45_80, 2_81, 45_75, 4_32, 2_96, 4_93, 1_94, L07, 2_48, 1_1, L88_1, 2_1, 1_2, 1_18, 2_42, 2_31, L10, L89_1, 3_14, 4_16, 42_59, 42_22, 3_6, 3_24, 3_40, 2_87, 1_87, 1_34, 2_10, 4_11, 42_9, L24_5, L27_3, 1_24, L72_1, 2_63, 2_88, 42_88, 3_1, L32_4, L81_5, 42_26, 4_31, 4_91, 1_51, 45_16, L62_2, 4_9, 3_12, 2_30, 2_38, 3_39, 2_32, L08, L86_5, L20_4, L45_4, L63, 1_10, 1_29, 1_40, L34, L59, 2_17, 3_18, 4_47, 2_23, 45_72, 2_27, 1_6, 45_17, 45_33, 45_91, L48_4, L44_1, L52, 2_25, 1_19, 1_27, 2_4, 2_24, 42_98, 42_80, 42_61, 45_5, 45_6, 45_76, L04_3, 4_33, 1_35, 1_23, 2_7, 42_93, L47_4, 3_23, 2_85, 45_31, 1_31, L78_2, 2_54, 1_54, L02_2, L60, 3_27, 2_43, 3_15, L28_5, L51, 45_51, 42_51, L80_5, 45_82, L85_2, 45_38, H13_4, L13_4, 42_37, 42_23, 42_31, L68_4, 1_17, 4_35, _4_4, 3_38, 45_66, 3_48, 42_42, 42_54, L43_2, 4_36, 2_45, 4_39, 2_47, 42_1, 42_70, 42_74, 3_10, 45_68, L03_5, L46_3, L95_3, 1_13, 1_22, 2_6, 42_48, 45_89, 42_36, 1_45, 3_56, 5_95, L54_5, 3_33, 4_41, _3_4, 3_47, L33_3, 3_8, 5_46, 5_35, 45_85, 45_27, 5_45, 42_16, 45_52, 1_14, 1_46, L06_4, 3_3, 5_75, 2_75, 2_35, 2_15, 42_91, 45_1, 45_2, 4_42, 1_20, 4_20, 1_39, 2_39, 42_15, 42_66, 42_4, 42_62, 42_47, 2_22, 45_77, 4_8, L23_4, 5_90, 5_34, L15_3, 1_9, 4_3, 4_43, L83_1, 1_37, 42_86, 45_81, 3_25, 3_41, 3_11, L87, 3_32, 42_75, L82_2, L30, L41_2, 3_28, 4_6, 5_92, 5_87, L79_4, 3_26, 45_60, 45_7, L29_2, 4_25, 3_34, 3_35, 4_23, 2_18, 2_19, L19_1, 1_33, 3_42, 4_34, 5_23, 5_69, L09, L49_5, 45_69, 5_88, 5_65, 42_39, 3_22, 4_24, 42_58, L17_5, 45_12, 42_41, 42_81, 42_69, 5_44, 5_15, 3_9, 1_26, 2_26, 1_30, 1_47, 42_87, 1_11, 2_37, 42_19, 42_52, 5_12, 5_83, 42_95, 2_5, 2_16, 45_83, L77_3, 42_76, 4_22, 1_4, 42_40, 4_2, 4_14, 45_73, 45_61, 45_63, 42_10, 42_94, 42_46, 45_37, 4_46, 42_55, 42_43, 45_41, 45_57, 45_94, 42_28, L90_3, L92_2, 45_34, 45_19, 42_89, 45_25, 45_49, 45_62, 42_85, 42_20, 3_2, 42_82, 1_42, 2_14, 4_17, 4_27, 1_21, 4_30, 5_96, 5_24, 42_90, 5_78, 5_16, 5_26, L57, 42_72, 42_96, 45_47, 5_93, 5_43, L75_3, 42_6, 45_39, 45_43, L21_1, L22_5, L76, 3_17, L36_4, 2_13, 45_10, 3_36, L25_1, 45_24, L96, L93, L67_5, 1_7, 45_14, L35, 45_78, 42_68, 45_59, 42_92, L50_3, 2_3, 45_11, 42_38, L12, 45_90, 42_30, 45_54, 45_20, 42_78, 45_74, 45_18, 45_86, 45_45, 1_5, 45_26, 45_30, 45_56, 2_12, 42_83, 2_21, L16_4, 4_13, 45_42, L65_3, L74, 5_28, 45_28, 42_79, 1_3, 45_50, 42_67, 45_35, L11_2, 5_98, L73, 42_45, 45_71, L31, 5_48, 45_48, L55, 3_20, 3_31, L91_5, 45_23, 45_36, 3_30, 42_63, 42_84, 45_44, 45_55, 1_12, and 5_18.

### DETAILED DESCRIPTION OF THE INVENTION

Provided herein are compositions and methods for the incorporation of modified nucleotides into a nucleic acid chain. Polymerases variously comprise DNA polymerases, RNA polymerases, template-independent polymerases, reverse transcriptases, or other enzyme capable of nucleotide extension. Reverse transcriptases are RNA/DNA-dependent DNA polymerases that have been shown to tolerate certain types of nucleotide modifications, such as modifications to the 3′ position of the sugar. This property makes reverse transcriptases a desirable protein engineering target to further enhance reversible terminator (removable chemical groups which prevent nucleic acid extension) incorporation for applications such as sequencing. Further provided herein are methods of sequencing employing mutant polymerases that incorporate modified nucleotides.

### Polypeptide Engineering

Provided herein are compositions and methods comprising mutant polypeptides relating to enzymes exhibiting increased incorporation of 3′ modified nucleotides. Mutations in the polymerases described herein variously comprise one or more changes to amino acid residues present in the polypeptide. Additions, substitutions, or deletions are all examples of mutations that are used to generate mutant polypeptides. Substitutions in some instances comprise the exchange of one amino acid for an alternative amino acid, and such alternative amino acids differ from the original amino acid with regard to size, shape, conformation, or chemical structure. Mutations in some instances are conservative or non-conservative. Conservative mutations comprise the substitution of an amino acid with an amino acid that possesses similar chemical properties. Additions often comprise the insertion of one or more amino acids at the N-terminal, C-terminal, or internal positions of the polypeptide. In some cases, additions comprise fusion polypeptides, wherein one or more additional polypeptides is connected to the polypeptide. Such additional polypeptides in some instances comprise domains with additional activity, or sequences with additional function (e.g., improve expression, aid purification, improve solubility, attach to a solid support, or other function). Often a polypeptide described herein comprises one or more non-amino acid groups. Fusion polypeptides optionally comprise an amino acid or other chemical linker that connects the one or more proteins. Any number of mutations is introduced into a polypeptide or portion of a polypeptide described herein, such as 1, 2, 3, 4, 5, 6, 7, 8, 9, 10, 20, 50, or more than 50 mutations.

In some cases, entire domains (portions of the polypeptide with a defined function) are added, deleted or substituted with domains from other polypeptides. Exemplary domains include DNA/RNA binding domains, nucleotide binding domains, nuclease domains, subcellular localization domains such as nuclear localization domains, or other domain. In some instances, one or more mutations are present in a catalytic site or binding domain. For example, a polypeptide comprises a reverse transcriptase-like binding domain comprising SEQ ID NO: 14 or a functional equivalent thereof. A domain in some cases comprises a DNA/RNA binding site, for example comprising residues at positions 63, 64, 97, 112-115, 118, 119, and 190 of SEQ ID NO: 14. Such sites are optionally found in analogous positions after alignment of other sequences to SEQ ID NO: 14. In other instances, a domain comprises an RNase (ribonuclease) domain comprising residues at SEQ ID NO: 15, or functional equivalent thereof. In some cases, a polypeptide comprises an active site. The active site of a polypeptide often comprises residues 149-154, 190, 191, 221, 223, 224, 168, and 169 of SEQ ID NO: 14. Such sites are often found at analogous positions in other domains (identified by aligning the two or more sequences for comparison), and polypeptides that comprise such domains are consistent with methods and compositions described herein.

Wild type sequences are often starting points for protein or enzyme engineering to generate mutant polypeptides. In some embodiments, a polypeptide differs from a wild-type sequence (naturally occurring) by at least one amino acid. Often a polypeptide differs by at least one amino acid from the nearest wild-type polypeptide. In some embodiments, a polypeptide differs from a wild-type sequence (naturally occurring) by at least two amino acids. In some embodiments, a polypeptide differs from a wild-type sequence (naturally occurring) by at least three, four, five, or at least six amino acids. Often, a wild type sequence is the closest wild type sequence, identified by aligning the polypeptide comprising at least one mutation with a wild type sequences.

Polypeptides described herein include but are not limited to polypeptides possessing enzymatic activity, such as polymerase activity, and are often described as families. Often, polymerases are DNA polymerases, RNA polymerases, template-independent polymerases, reverse transcriptases, or other enzyme capable of nucleotide extension. Many reverse transcriptases are known in the art, and such enzymes in some instances are mutated to generate the compositions described herein. Members of the reverse transcriptase family are often defined in terms of polymerase activity, active site structure, domain homology/function, or sequence homology to other known reverse transcriptase family members. For example, reverse transcriptases include but are not limited to HIVRT, MMLV, AVN or other member of the reverse transcriptase family. In some instances, SEQ ID Nos.: 1, 2, 3, 4, 17, and 18 are members of the reverse transcriptase family. Wild-type reverse transcriptases are obtained from any number of origins, such as eukaryotic, prokaryotic, or viral origins.

Further provided herein are polypeptides comprising a sequence that has at least 85% identity with SEQ ID NO: 1 or 2 and at least one mutation at K152, D153, A154, F155, F156, Q190 (SEQ ID NO: 1 numbering). In some cases, a polypeptide described herein comprises a sequence that has at least 70%, 75%, 80%, 85%, 90%, 95%, or at least 97% identity with SEQ ID NO: 1 or 2 and at least one mutation at K152, D153, A154, F155, F156, Q190 (SEQ ID NO: 1 numbering). Often a polypeptide described herein will comprise at least one mutation not found in the wild type polypeptide. A polypeptide that has at least 70%, 75%, 80%, 85%, 90%, 95%, or at least 97% identity with SEQ ID NOs: 1, 2, 3, 17, or 18 and having at least one mutation at a position analogous to K152, D153, A154, F155, F156, Q190 is further described herein.

Exemplary amino acid sequences corresponding to the compositions and methods described herein are shown in Table 1.

TABLE 1

| SEQ | | |
|---|---|---|
| ID NO | Name | Amino Acid Sequence |
| 1 | MMLV | MLNIEDEHRLHETSKEPDVSLGSTWLSDFPQAWAETGG |
| | (Murine Moloney | MGLAVRQAPLIIPLKATSTPVSIKQYPMSQEARLGIKPHIQ |
| | Reverse | RLLDQGILVPCQSPWNTPLLPVKKPGTNDYRPVQDLREV |
| | Transcriptase) | NKRVEDIHPTVPNPYNLLSGLPPSHQWYTVLDLKDAFFC |
| | | LRLHPTSQPLFAFEWRDPEMGISGQLTWTRLPQGFKNSP |
| | | TLFDEALHRDLADFRIQHPDLILLQYVDDLLLAATSELDC |
| | | QQGTRALLQTLGNLGYRASAKKAQICQKQVKYLGYLLK |
| | | EGQRWLTEARKETVMGQPTPKTPRQLREFLGTAGFCRL |
| | | WIPGFAEMAAPLYPLTKTGTLFNWGPDQQKAYQEIKQA |
| | | LLTAPALGLPDLTKPFELFVDEKQGYAKGVLTQKLGPW |
| | | RRPVAYLSKKLDPVAAGWPPCLRMVAAIAVLTKDAGKL |
| | | TMGQPLVILAPHAVEALVKQPPDRWLSNARMTHYQALL |
| | | LDTDRVQFGPVVALNPATLLPLPEEGLQHNCLDILAEAH |
| | | GTRPDLTDQPLPDADHTWYTDGSSLLQEGQRKAGAAVT |
| | | TETEVIWAKALPAGTSAQRAELIALTQALKMAEGKKLN |
| | | VYTDSRYAFATAHIHGEIYRRRGLLTSEGKEIKNKDEILA |
| | | LLKALFLPKRLSIIHCPGHQKGHSAEARGNRMADQAARK |
| | | AAITETPDTSTLLI (SEQ ID NO: 1) |
| 2 | MMLV-ΔN23 | MSTWLSDFPQAWAETGGMGLAVRQAPLIIPLKATSTPVSIKQYPMSQ |
| | | EARLGIKPHIQRLLDQGILVPCQSPWNTPLLPVKKPGTNDYRPVQDL |
| | | REVNKRVEDIHPTVPNPYNLLSGLPPSHQWYTVLDLKDAFFCLRLHP |
| | | TSQPLFAFEWRDPEMGISGQLTWTRLPQGFKNSPTLFDEALHRDLAD |
| | | FRIQHPDLILLQYVDDLLLAATSELDCQQGTRALLQTLGNLGYRASA |
| | | KKAQICQKQVKYLGYLLKEGQRWLTEARKETVMGQPTPKTPRQLR |
| | | EFLGTAGFCRLWIPGFAEMAAPLYPLTKTGTLFNWGPDQQKAYQEI |
| | | KQALLTAPALGLPDLTKPFELFVDEKQGYAKGVLTQKLGPWRRPVA |
| | | YLSKKLDPVAAGWPPCLRMVAAIAVLTKDAGKLTMGQPLVILAPHA |
| | | VEALVKQPPDRWLSNARMTHYQALLLDTDRVQFGPVVALNPATLLP |
| | | LPEEGLQHNCLDILAEAHGTRPDLTDQPLPDADHTWYTDGSSLLQEG |
| | | QRKAGAAVTTETEVIWAKALPAGTSAQRAELIALTQALKMAEGKKL |
| | | NVYTDSRYAFATAHIHGEIYRRRGLLTSEGKEIKNKDEILALLKALFL |
| | | PKRLSIIHCPGHQKGHSAEARGNRMADQAARKAAITETPDTSTLLI |
| | | (SEQ ID NO: 2) |
| 3 | HIVRT | PISPIETVPVKLKPGMDGPKVKQWPLTEEKIKALVEICIEMEKEGKIS |
| | (HIV Reverse | KIGPENPYNTPVFAIKKKDSTKWRKLVDFRELNKRTQDFWEVQLGIP |
| | Transcriptase) | HPAGLKKKKSVTVLDVGDAYFSVPLDEDFRKYTAFTIPSINNETPGIR |
| | | YQYNVLPQGWKGSPAIFQSSMTKILEPFRKQNPDIVIYQYMDDLYVG |
| | | SDLEIGQHRTKIEELRQHLLRWGLTTPDKKHQKEPPFLWMGYELHPD |
| | | KWTVQPIVLPEKDSWTVNDIQKLVGKLNWASQTYPGIKVRQLCKLL |
| | | RGTKALTEVIPLTEEAELELAENREILKEPVHGVYYDPSKDLIAEIQK |
| | | QGQGQWTYQIYQEPFKNLKTGKYARMRGAITINDVKQLIEAVQKIT |
| | | TESIVIWGKTPKFKLPIQKETWETWWTEYWQATWIPEWEFVNTPPLV |
| | | KLWYQLEKEPIVGAETF (SEQ ID NO: 3) |
| 4 | AMVRT | TVALHLAIPLKWKPNHTPVWIDQWPLPEGKLVALTQLV |
| | (Avian | EKELQLGHIEPSLSCWNTPVFVIRKASGSYRLLHDLRAV |
| | Myeoblastosis | NAKLVPFGAVQQGAPVLSALPRGWPLMVLDLKDCFFSIP |
| | Virus Reverse | LAEQDREAFAFTLPSVNNQAPARRFQWKVLPQGMTCSP |
| | Transcriptase) | TICQLIVGQILEPLRLKHPSLRMLHYMDDLLLAASSHDGL |
| | | EAAGEEVISTLERAGFTISPDKVQREPGVQYLGYKLGSTY |
| | | VAPVGLVAEPRIATLWDVQKLVGSLQWLRPALGIPPRL |
| | | MGPFYEQLRGSDPNEAREWNLDMKMAWREIVQLSTTA |
| | | ALERWDPALPLEGAVARCEQGAIGVLGQGLSTHPRPCL |
| | | WLFSTQPTKAFTAWLEVLTLLITKLRASAVRTFGKEVDIL |
| | | LLPACFREDLPLPEGILLALRGFAGKIRSSDTPSIFDIARPL |
| | | HVSLKVRVTDHPVPGPTVFTDASSSTHKGVVVWREGPR |
| | | WEIKEIADLGASVQQLEARAVAMALLLWPTTPTNVVTD |
| | | SAFVAKMLLKMGQEGVPSTAAAFILEDALSQRSAMAAV |
| | | LHVRSHSEVPGFFTEGNDVADSQATFQAYPLREAKDLHT |
| | | ALHIGPRALSKACNISMQQAREVVQTCPHCNSAPALEAG |
| | | VNPRGLGPLQIWQTDFTLEPRMAPRSWLAVTVDTASSAI |
| | | VVTQHGRVTSVAAQHHWATAIAVLGRPKAIKTDNGSCF |
| | | TSKSTREWLARWGIAHTTGIPGNSQGQAMVERANRLLK |
| | | DKIRVLAEGDGFMKRIPTSKQGELLAKAMYALNHFERG |
| | | ENTKTPIQKHWRPTVLTEGPPVKIRIETGEWEKGWNVLV |
| | | WGRGYAAVKNRDTDKVIWVPSRKVKPDIAQKDEVTKK |
| | | DEASPLFA (SEQ ID NO: 4) |
| 5 | MMLV-K152R | WYTVLDLRDAFFCLRL (SEQ ID NO: 5) |
| 6 | MMLV-K152C | LDLCDAFF (SEQ ID NO: 6) |
| 7 | MMLV-K152S | VLDLSDAFF (SEQ ID NO: 7) |
| 8 | MMLV-K152L | VLDLLDAFF (SEQ ID NO: 8) |
| 9 | MMLV-D153Y | TVLDLKYAFFCLRL (SEQ ID NO: 9) |
| 10 | MMLV-A154Y | LKDYFFC (SEQ ID NO: 10) |
| 11 | MMLV-F155A | LKDAAFCL (SEQ ID NO: 11) |
| 12 | MMLV-F156N | VLDLKDAFNCLRLHP (SEQ ID NO: 12) |
| 13 | MMLV-Q190A | RLPAGFK (SEQ ID NO: 13) |
| 14 | MMLV-Q190S | RLPSGFKN (SEQ ID NO: 14) |
| 15 | MMLV-ZFREV | PVSIKQYPMSQEARLGIKPHIQRLLDQGILVPCQSPWNTPLLPVKKPG |
| | | TNDYRPVQDLREVNKRVEDIHPTVPNPYNLLSGLPPSHQWYTVLDL |
| | | KDAFFCLRLHPTSQPLFAFEWRDPEMGISGQLTWTRLPQGFKNSPTL |
| | | FDEALHRDLADFRIQHPDLILLQYVDDLLLAATSELDCQQGTRALLQ |
| | | TLGNLGYRASAKKAQICQKQVKYLGYLL (SEQ ID NO: 15) |
| 16 | MMLV- | TWYTDGSSLLQEGQRKAGAAVTTETEVIWAKALPAGTSAQRAELIA |
| | RNase_HI_RT_Be1 | LTQALKMAEGKKLNVYTDSRYAFATAHIHGEIYRRRGLLTSEGKEIK |
| | | NKDEILALLKALFLPKRLSIIHCPGHQKGHSAEARGNRMADQAARKA |
| | | A (SEQ ID NO: 16) |
| 17 | TFRT | SSGLVPRGSHMTLNIEDEHRLHETSKEPDVSLGSTWLSDFPQAWAET |
| | (Tumebacillus | GGMGLAVRQAPLIIPLKATSTPVSIKQYPMSQKARLGIKPHIQRLLDQ |
| | flagellates_RT) | GILVPCQSPWNTPLLPVKKPGTNDYRPVQDLREVNKRVEDIHPTVPN |
| | | PYNLLSGLPPSHQWYTVLDLKDAFFCLRLHPTSQPLFAFEWRDPEMG |
| | | ISGQLTWTRLPQGFKNSPTLFDEALHRDLADFRIQHPDLILLQYVDDL |
| | | LLAATSELDCQQGTRALLQTLGNLGYRASAKKAQICQKQVKYLGYL |
| | | LKEGQRWLTEARKETVMGQPTPKTPRQLRRFLGTAGFCRLFIPGFAE |
| | | MAAPLYPLTKTGTLFNWGPDQQKAYQEIKQALLTAPALGLPDLTKP |
| | | FELFVDEKQGYAKGVLTQKLGPWRRPVAYLSKKLDPVAAGWPPCL |
| | | RMVAAIAVLTKDAGKLTMGQPLVIGAPHAVEALVKQPPDRWLSKA |
| | | RMTHYQALLLDTDRVQFGPVVALNPATLLPLPEEGLQHNCLDILAEA |
| | | HGTRPDLTDQPLPDADHTWYTNGSSLLQEGQRKAGAAVTTETEVIW |
| | | AKALPAGTSAQRAELIALTQALKMAEGKKLNVYTDSRYAFATAHIH |
| | | GEIYRRRGLLTSEGKEIKNKDEILALLKALFLPKRLSIIHCPGHQKGHS |
| | | AEARGNRMADQAARKAAITET (SEQ ID NO: 17) |
| 18 | FLV (feline | TLQLEEEYRLFEPESTQKQEMDIWLKNFPQAWAETGGMGTAHCQAP |
| | leukemia virus) | VLIQLKATATPISIRQYPMPHEAYQGIKPHIRRMLDQGILKPCQSPWN |
| | | TPLLPVKKPGTEDYRPVQDLREVNKRVEDIHPTVPNPYNLLSTLPPSH |
| | | PWYTVLDLKDAFFCLRLHSESQLLFAFEWRDPEIGLSGQLTWTRLPQ |
| | | GFKNSPTLFDEALHSDLADFRVRYPALVLLQYVDDLLLAAATRTECL |
| | | EGTKALLETLGNKGYRASAKKAQICLQEVTYLGYSLKDGQRWLTK |
| | | ARKEAILSIPVPKNSRQVREFLGTAGYCRLWIPGFAELAAPLYPLTRP |
| | | GTLFQWGTEQQLAFEDIKKALLSSPALGLPDITKPFELFIDENSGFAK |
| | | GVLVQKLGPWKRPVAYLSKKLDTVASGWPPCLRMVAAIAILVKDA |
| | | GKLTLGQPLTILTSHPVEALVRQPPNKWLSNARMTHYQAMLLDAER |
| | | VHFGPTVSLNPATLLPLPSGGNHHDCLQILAETHGTRPDLTDQPLPDA |
| | | DLTWYTDGSSFIRNGEREAGAAVTTESEVIWAAPLPPGTSAQRAELI |
| | | ALTQALKMAEGKKLTVYTDSRYAFATTHVHGEIYRRRGLLTSEGKEI |
| | | KNKNEILALLEALFLPKRLSIIHCPGHQKGDSPQAKGNRLADDTAKK |
| | | AATETHSSLTVL (SEQ ID NO: 18) |
| 19 | MMLV | MTWLSDFPQAWAETGGMGLAVRQAPLIIPLKATSTPVSIKQYPMSQE |
| | | ARLGIKPHIQRLLDQGILVPCQSPWNTPLLPVKKPGTNDYRPVQDLR |
| | | EVNKRVEDIHPTVPNPYNLLSGLPPSHQWYTVLDLKDAFFCLRLHPT |
| | | SQPLFAFEWRDPEMGISGQLTWTRLPQGFKNSPTLFDEALHRDLADF |
| | | RIQHPDLILLQYVDDLLLAATSELDCQQGTRALLQTLGNLGYRASAK |
| | | KAQICQKQVKYLGYLLKEGQRWLTEARKETVMGQPTPKTPRQLREF |
| | | LGTAGFCRLWIPGFAEMAAPLYPLTKTGTLFNWGPDQQKAYQEIKQ |
| | | ALLTAPALGLPDLTKPFELFVDEKQGYAKGVLTQKLGPWRRPVAYL |
| | | SKKLDPVAAGWPPCLRMVAAIAVLTKDAGKLTMGQPLVIKAPHAV |
| | | EALVKQPPDRWLSNARMTHYQALLLDTDRVQFGPVVALNPATLLPL |
| | | PEEGLQHNCLDILAEAHGTRPDLTDQPLPDADHTWYTDGSSLLQEG |
| | | QRKAGAAVTTETEVIWAKALPAGTSAQRAALIALTQALKMAEGKKL |
| | | NVYTASRYAFATAHIHGEIYRRRGLLTSEGKEIKNKDEILALLKALFL |
| | | PKRLSIIHCPGHQKGHSAEARGNRMADQAARKAAITETPDTSTLLI |
| | | (SEQ ID NO: 19) |
| 20 | XMRV (Xenotropic | DFPQAWAETGGMGLAVRQAPLIIPLKATSTPVSIKQYPMSQEARLGI |
| | murine leukemia | KPHIQRLLDQGILVPCQSPWNTPLLPVKKDYRPVQDLREVNKRVEDI |
| | virus-related virus) | HPTVPNPYNLLSGLPPSHQWYTVLDLKDAFFCLRLHPTSQPLFAFEW |
| | | RDQLTWTRLPQGFKNSPTLFDEALHRDLADFRIQHPDLILLQYVDDL |
| | | LLAATSEQDCQRGTRALLQTLGNLGYRASAKKAQICQKQVKYLGYL |
| | | LKEGQRWLTEARKETVMGQPTPKTPRQLREFLGTAGFCRLWIPGFA |
| | | EMAAPLYPLTKTLFNWGPDQQKAYQEIKQALLTAPALGLPDLTKPFE |
| | | LFVDEKQGYAKGVLTQKLGPWRRPVAYLSKKLDPVAAGWPPCLRM |
| | | VAAIAVLTKDAGKLTMGQPLVILAPHAVEALVKQPPARMTHYQAM |
| | | LLDTDRVQFGPVVALNPATLLPLP (SEQ ID NO: 20) |
| 21 | 4mh8 | TWLSDFPQAWAETGGMGLAVRQAPLIIPLKATSTPVSIKQYPMSQEA |
| | | RLGIKPHIQRLLDQGILVPCQSPWNTPLLPVKTNDYRPVQDLREVNK |
| | | RVEDIHPTVPNPYNLLSGLPPSHQWYTVLDLKDAFFCLRLHPTSQPLF |
| | | AFEWRDPEMGISGQLTWTRLPQGFKNSPTLFDEALHRDLADFRIQHP |
| | | DLILLQYVDDLLLAATSELDCQQGTRALLQTLGNLGYRASAKKAQIC |
| | | QKQVKYLGYLLKEGQRWLTEARKETVMGQPTPKTPRQLREFLGTA |
| | | GFCRLWIPGFAEMAAPLYPLTKTLFNWGPDQQKAYQEIKQALLTAP |
| | | ALGLPDLTKPFELFVDEKQGYAKGVLTQKLGPWRRPVAYLSKKLDP |
| | | VAAGWPPCLRMVAAIAVLTKDAGKLTMGQPLVIKAPHAVEALVKQ |
| | | PPARMTHYQALLLDTDRVQFGPVVALNPATLLPLP (SEQ ID NO: 21) |
| 22 | FIV (feline | QIKQWPLTNEKIEALTEIVERLEREGKVKRADPNNPWNTPVFAIKKK |
| | immunodeficiency | SGKWRMLIDFRELNKLIEKGAQLGLPHPAGLQIKKQVTVLDIGDAY |
| | virus) | FTIPLDPDYAPYTAFTLPRKNNAGPGRRFVWCSLPQGWILSPLIYQST |
| | | LDNIIQPFIRQNPQLDIYQYMDDIYIGSNLSKKEHKEKVEELRKLLLW |
| | | WGFETPEDKLQEEPPYTWMGYELHPLTWTIQQKQLDIPEQPTLNELQ |
| | | KLAGKINWASQAIPDLSIKALTNMMRGNQNLNSTRQWTKEARLEVQ |
| | | KAKKAIEEQVQLGYYDPSKELYAKLSLVGPHQISYQVYQKDPEKIL |
| | | WYGKMSRQKKKAENTCDIALRACYKIREESIIRIGKEPRYEIPTSREA |
| | | WESNLINSPYLKAPPPEVEYIHAALNIKRALSMIKDAPIPGAETWYID |
| | | GGRKLGKAAKAAYWTDTGKWQVMELEGSNQKAEIQALLLALKAG |
| | | SEEMNIITDSQYVINIILQQPDMMEGIWQEVLEELEKKTAIFIDWVPG |
| | | HKGIPGNEEVDKLCSDKIPVVKVKMKDPNKGPQIKQWPLTNEKIEAL |
| | | TEIVERLEREGKVKRADPNNPWNTPVFAIKKKSGKWRMLIDFRELN |
| | | KLIEKLGLPHPAGLQIKKQVTVLDIGDAYFTIPLDPDYAPYTAFTLPR |
| | | KNNAGPGRRFVWCSLPQGWILSPLIYQSTLDNIIQPFIRQNPQLDIYQY |
| | | MDDIYIGSNLSKKEHKEKVEELRKLLLWPEDKLQEETWTIQQKQLDI |
| | | PEQPTLNELQKLAGKINWASQAIPDLSIKALTNMMRGNQNLNSTRQ |
| | | WTKEARLEVQKAKKAIEEQVQLGYYDPSKELYAKLSLVGPHQISYQ |
| | | VYQKDPEKILWYGKMSRQKKKAENTCDIALRACYKIREESIIRIGKEP |
| | | RYEIPTSREAWESNLINSPYLKAPPPEVEYIHAALNIKRALS (SEQ ID |
| | | NO: 22) |
| 23 | HIV2 | AKVEPIKIMLKPGKDGPKLRQWPLTKEKIEALKEICEKMEKEGQLEE |
| | | APPTNPYNTPTFAIKKKDRMLIDFRELNKVTQDFTEIQLGIPHPAGLA |
| | | KKRRITVLDVGDAYFSIPLHEDFRPYTAFTLKRYIYKVLPQGWKGSP |
| | | AIFQHTMRQVLEPFRKANKDVIIIQYMDDILIASDRTDLEHDRVVLQL |
| | | KELLNGLGFSTPDEKFQKDPPYHWMGYELWPTKWKLQKIQLPQKEI |
| | | WTVNDIQKLVGVLNWAAQLYPGIKTKHLCRLISGKMTLTEEVQWTE |
| | | LAEAELEENRIILSQEQEGHYYQEEKELEATVQKDQDNQWTYKIHQE |
| | | EKILKVGKYAKVTHTNGIRLLAQVVQKIGKEALVIWGRIPKFHLPVE |
| | | REIWEQWWDNYWQVTWIPDWDFVSTPPLVRLAFNLVGDPIPGAETF |
| | | YTDGSCNRQSKEGKAGYVTDRGKDKVKKLEQTTNQQAELEAFAMA |
| | | LTDSGPKVNIIVDSQYVMGIVASQPIESESKIVNQIIEEMIKKEAIYVA |
| | | WVPAHKGIGGNQEVDHLVSQGIEPIKIMLKPGKDGPKLRQWPLTKE |
| | | KIEALKEICEKMEKEGQLEEAPPTNPYNTPTFAIKNKWRMLIDFRELN |
| | | KVTQDFTEIQPHPAGLAKKRRITVLDVGDAYFSIPLHEDFRPYTAFTL |
| | | PSVNNAEPGKRYIYKVLPQGWKGSPAIFQHTMRQVLEPFRKANKDVI |
| | | IIQYMDDILIASDRTDLEHDRVVLQLKELLNGWMGYELWPTKWKLQ |
| | | KIQLPQKEIWTVNDIQKLVGVLNWAAQLYPGIKTKHLCRLISGKMTL |
| | | IEEVQWIELAEAELEENRIILSQEQEGHYYQEEKELEATVQKDQDNQ |
| | | WTYKIHQEEKILKVGKYAKVKNTHTNGIRLLAQVVQKIGKEALVIW |
| | | GRIPKFHLPVEREIWEQWWDNYWQVTWIPDWDFVSTPPLVRLAFNL |
| | | VGD (SEQ ID NO: 23) |
| 24 | HIV Type O | PISPIAPVPVKLKPGMDGPKVKQWPLSKEKIEALTAICQEMEQEGKIS |
| | Reverse | RIGPENPYNTPIFAIKKKDSTKWRKLVDFRELNKRTQDFWEVQLGIP |
| | Transcriptase | HPGGLKQKRSVTVLDVGDAYFSCPLDPDFRKYTAFTIPSVNNETPGIR |
| | | YQYNVLPQGWKGSPAIFQSSMTKILDPFRKDNPELEICQYMDDLYVG |
| | | SDLPLAEHRKRVELLREHLYQWGFTTPDKKHQKEPPFMWMGYELH |
| | | PDKWTVQPIKLPNKDVWTVNDIQKLIGKLNWASQIYQGIRVRELCKL |
| | | IRGTKSLIEVVPLSKEAEMELEENREKLKEPMHGVYYQPDKDLWVN |
| | | IQKQGEGQWTYQIYQDEHKNLKTGKYTRQRGAFFINDIRQLAEVIQK |
| | | VSQESIVIWGKLPKFKLPVTRETWETWWADYWQATWIPEWDYVST |
| | | PPLIKLWYRLESEPIMGAETYYVDGAANRDTKLGKAGYVTEQGKQK |
| | | IIKLNETTNQKAELMAVLLALQDSKEKVNIVTDSQYVLGIISSQPTQS |
| | | ESPIVQQIIEELTKKEQVYLTWVPAHKGIGGNEKIDKLVSKDIRRVL |
| | | (SEQ ID NO: 24) |
| 25 | HIV1 | MVPISPIETVPVKLKPGMDGPKVKQWPLIEEKIKALVEICIEMEKEG |
| | | KISKIGPENPYNTPVFAIKKKDSTKWRKLVDFRELNKRTQDFWEVQL |
| | | GIPHPAGLKKKKSVTVLDVGDAYFSVPLDEDFRKYTAFTIPSINNETP |
| | | GIRYQYNVLPQGWKGSPAIFQSSMTKILEPFKKQNPDIVIYQYMDDL |
| | | YVGSDLEIGQHRTKIEELRQHLLRWGLTTPDKKHQKEPPFLWMGYE |
| | | LHPDKWTVQPIVLPEKDSWTVNDICKLVGKLNWASQIYPGIKVRQLS |
| | | KLLRGTKALIEVIPLTEEAELELAENREILKEPVHGVYYDPSKDLIAEI |
| | | QKQGQGQWTYQIYQEPFKNLKTGKYARMRGAHTNDVKQLTEAVQ |
| | | KITTESIVIWGKTPKFKLPIQKETWETWWIEYWQATWIPEWEFVNTP |
| | | PLVKLWYQLEKEPIVGAETFYVDGAANRETKLGKAGYVTNKGRQK |
| | | VVPLTNTTNQKIELQATYLALQDSGLEVNIVTNSQYALGIIQAQPDKS |
| | | ESELVNQIIEQLIKKEKVYLAWVPAHKGIGGNEQVDKLVSAPIETVPV |
| | | KLKPGMDGPKVKQWPLIEEKIKALVEICIEMEKEGKISKIGPENPYN |
| | | TPVFAIKKKDSTKWRKLVDFRELNKRTQDFWEVQLGIPHPAGLKKK |
| | | KSVTVLDVGDAYFSVPLDEDFRKYTAFTIPSINNETPGIRYQYNVLPQ |
| | | GWKGSPAIFQSSMTKILEPFKKQNPDIVIYQYMDDLYVGSDLEIGQH |
| | | RTKIEELRQHLLRWGLTTPGYELHPDKWTVQPIVLPEKDSWTVNDIQ |
| | | KLVGKLNWASQIYPGIKVRQLSKLLRGTKALTEVIPLTEEAELELAEN |
| | | REILKEPVHGVYYDPSKDLIAEIQKQGQGQWTYQIYQEPFKNLKTGK |
| | | YARMRGAHTNDVKQLTEAVQKITTESIVIWGKTPKFKLPIQKETWET |
| | | WWIEYWQATWIPEWEFVNTPPLVKLWYQ (SEQ ID NO: 25) |

Further described herein are segments, or portions of a larger polypeptide. Optionally, segments have catalytic activity such as nucleic acid extension activity, particularly in the context of a reverse transcriptase domain or polymerase domain as described herein. Described herein are polypeptides comprising any one of the segments of SEQ ID NOs: 4-16, and at least one additional residue at the N or C terminus (+1 residue). In some instances both the N and C terminus has at least an additional residue, two, three four five, six seven, eight, nine, ten 11, 12, 13, 14, 15, 16, 17, 18, 19, 20, 21, 22, 23, 24, 25, 26, 27, 28, 29, 30, 35, 40, 45, 50, 60, 70, 80, 90, 100, or more than 100 additional residues. For example, described herein are polypeptides comprising SEQ ID NO: 4(+1 residue), such as an adjacent N-terminal aspartic acid, an adjacent C-terminal arginine, or a combination thereof, or additional residues such as residues identified through an alignment of SEQ ID NO: 4 to SEQ ID NO:1, accounting for a single mutated residue or other residues contributed to a polypeptide comprising the SEQ ID NO. Described herein are polypeptides comprising SEQ ID NO: 5(+1 residue), such as an adjacent N-terminal glutamine, an adjacent C-terminal histidine, or a combination thereof, or additional residues such as residues identified through an alignment of SEQ ID NO: 5 to SEQ ID NO: 1, accounting for a single mutated residue, or other residues contributed to a polypeptide comprising the SEQ ID NO. Described herein are polypeptides comprising SEQ ID NO: 6(+1 residue), such as an adjacent N-terminal valine, an adjacent C-terminal cysteine, or a combination thereof, or additional residues such as residues identified through an alignment of SEQ ID NO: 6 to SEQ ID NO:1, accounting for a single mutated residue, or other residues contributed to a polypeptide comprising the SEQ ID NO. Described herein are polypeptides comprising SEQ ID NO: 7(+1 residue), such as an adjacent N-terminal threonine, an adjacent C-terminal cysteine, or a combination thereof, or additional residues such as residues identified through an alignment of SEQ ID NO: 7 to SEQ ID NO: 1, accounting for a single mutated residue, or other residues contributed to a polypeptide comprising the SEQ ID NO. Described herein are polypeptides comprising SEQ ID NO: 8(+1 residue), such as an adjacent N-terminal threonine, an adjacent C-terminal cysteine, or a combination thereof, or additional residues such as residues identified through an alignment of SEQ ID NO: 8 to SEQ ID NO:1, accounting for a single mutated residue, or other residues contributed to a polypeptide comprising the SEQ ID NO. Described herein are polypeptides comprising SEQ ID NO: 9(+1 residue), such as an adjacent N-terminal aspartic acid, an adjacent C-terminal leucine, or a combination thereof, or additional residues such as residues identified through an alignment of SEQ ID NO: 9 to SEQ ID NO:1, accounting for a single mutated residue, or other residues contributed to a polypeptide comprising the SEQ ID NO. Described herein are polypeptides comprising SEQ ID NO: 10(+1 residue), such as an adjacent N-terminal aspartic acid, an adjacent C-terminal arginine, or a combination thereof, or additional residues such as residues identified through an alignment of SEQ ID NO:10 to SEQ ID NO:1, accounting for a single mutated residue, or other residues contributed to a polypeptide comprising the SEQ ID NO. Described herein are polypeptides comprising SEQ ID NO: 11(+1 residue), such as an adjacent N-terminal threonine, an adjacent C-terminal threonine, or a combination thereof, or additional residues such as residues identified through an alignment of SEQ ID NO:11 to SEQ ID NO: 1, accounting for a single mutated residue, or other residues contributed to a polypeptide comprising the SEQ ID NO. Described herein are polypeptides comprising SEQ ID NO: 12(+1 residue), such as an adjacent N-terminal threonine, an adjacent C-terminal asparagine, or a combination thereof, or additional residues such as residues identified through an alignment of SEQ ID NO: 12 to SEQ ID NO:1, accounting for a single mutated residue, or other residues contributed to a polypeptide comprising the SEQ ID NO. Described herein are polypeptides comprising SEQ ID NO: 13(+1 residue), such as an adjacent N-terminal threonine, an adjacent C-terminal asparagine, or a combination thereof, or additional residues such as residues identified through an alignment of SEQ ID NO:13 to SEQ ID NO:1, accounting for a single mutated residue, or other residues contributed to a polypeptide comprising the SEQ ID NO. Described herein are polypeptides comprising SEQ ID NO: 14(+1 residue), such as an adjacent N-terminal threonine, an adjacent C-terminal serine, or a combination thereof, or additional residues such as residues identified through an alignment of SEQ ID NO: 14 to SEQ ID NO:1, accounting for a single mutated residue, or other residues contributed to a polypeptide comprising the SEQ ID NO.

Exemplary polypeptide mutants described herein are shown in Table 2. In some instances, a polypeptide described herein has a sequence that has at least 85% identity to SEQ ID NO: 1 or 2, and that further exhibits at least one of the mutations shown in Table 2. In some cases, a polypeptide described herein has a sequence that has at least 70%, 75%, 80%, 85%, 90%, 91%, 92%, 93%, 94%, 95%, 96%, 97%, 98%, 99%, or greater than 99% identity to SEQ ID NO: 1 or 2, and that further exhibits at least one of the mutations shown in Table 2. Some polypeptides described herein has a sequence exhibiting substantial identity, such as at least 70%, 75%, 80%, 85%, 90%, 91%, 92%, 93%, 94%, 95%, 96%, 97%, 98%, 99%, or greater than 99% identity to a polypeptide of SEQ ID NO: 1, 2, 3, 4, 17, or 18 and exhibits at least one of the mutations shown in Table 2. Additional polypeptides contemplated and disclosed herein comprise a reverse transcriptase domain having at least one mutation at a position analogous to at least one of the positions in Table 2, up to and including all of the positions indicated in Table 2, in some cases to come to polypeptides having one or more of the mutations indicated in Table 2 at a homologous position.

TABLE 2

| | Position relative to SEQ ID NO: 1 | | | | | | |
|---|---|---|---|---|---|---|---|
| # | K152 | D153 | A154 | F155 | F156 | Q190 | Mutations relative to SEQ ID NO: 1 |
| — | K | D | A | F | F | Q | Wildtype |
| 1 | K | D | A | F | F | A | Q190A |
| 2 | K | D | A | F | F | S | Q190S |
| 3 | K | D | A | F | N | Q | F156N |
| 4 | K | D | A | F | N | A | F156N; Q190A |
| 5 | K | D | A | F | N | S | F156N; Q190S |
| 6 | K | D | A | A | F | Q | F155A |
| 7 | K | D | A | A | F | A | F155A; Q190A |
| 8 | K | D | A | A | F | S | F155A; Q190S |
| 9 | K | D | A | A | N | Q | F155A; F156N |
| 10 | K | D | A | A | N | A | F155A; F156N; Q190A |
| 11 | K | D | A | A | N | S | F155A; F156N; Q190S |
| 12 | K | D | Y | F | F | Q | A154Y |
| 13 | K | D | Y | F | F | A | A154Y; Q190A |
| 14 | K | D | Y | F | F | S | A154Y; Q190S |
| 15 | K | D | Y | F | N | Q | A154Y; F156N |
| 16 | K | D | Y | F | N | A | A154Y; F156N; Q190A |
| 17 | K | D | Y | F | N | S | A154Y; F156N; Q190S |
| 18 | K | D | Y | A | F | Q | A154Y; F155A |
| 19 | K | D | Y | A | F | A | A154Y; F155A; Q190A |
| 20 | K | D | Y | A | F | S | A154Y; F155A; Q190S |
| 21 | K | D | Y | A | N | Q | A154Y; F155A; F156N |
| 22 | K | D | Y | A | N | A | A154Y; F155A; F156N; Q190A |
| 23 | K | D | Y | A | N | S | A154Y; F155A; F156N; Q190S |
| 24 | K | Y | A | F | F | Q | D153Y |
| 25 | K | Y | A | F | F | A | D153Y; Q190A |
| 26 | K | Y | A | F | F | S | D153Y; Q190S |
| 27 | K | Y | A | F | N | Q | D153Y; F156N |
| 28 | K | Y | A | F | N | A | D153Y; F156N; Q190A |
| 29 | K | Y | A | F | N | S | D153Y; F156N; Q190S |
| 30 | K | Y | A | A | F | Q | D153Y; F155A |
| 31 | K | Y | A | A | F | A | D153Y; F155A; Q190A |
| 32 | K | Y | A | A | F | S | D153Y; F155A; Q190S |
| 33 | K | Y | A | A | N | Q | D153Y; F155A; F156N |
| 34 | K | Y | A | A | N | A | D153Y; F155A; F156N; Q190A |
| 35 | K | Y | A | A | N | S | D153Y; F155A; F156N; Q190S |
| 36 | K | Y | Y | F | F | Q | D153Y; A154Y |
| 37 | K | Y | Y | F | F | A | D153Y; A154Y; Q190A |
| 38 | K | Y | Y | F | F | S | D153Y; A154Y; Q190S |
| 39 | K | Y | Y | F | N | Q | D153Y; A154Y; F156N |
| 40 | K | Y | Y | F | N | A | D153Y; A154Y; F156N; Q190A |
| 41 | K | Y | Y | F | N | S | D153Y; A154Y; F156N; Q190S |
| 42 | K | Y | Y | A | F | Q | D153Y; A154Y; F155A |
| 43 | K | Y | Y | A | F | A | D153Y; A154Y; F155A; Q190A |
| 44 | K | Y | Y | A | F | S | D153Y; A154Y; F155A; Q190S |
| 45 | K | Y | Y | A | N | Q | D153Y; A154Y; F155A; F156N |
| 46 | K | Y | Y | A | N | A | D153Y; A154Y; F155A; F156N; Q190A |
| 47 | K | Y | Y | A | N | S | D153Y; A154Y; F155A; F156N; Q190S |
| 48 | R | D | A | F | F | Q | K152R |
| 49 | R | D | A | F | F | A | K152R; Q190A |
| 50 | R | D | A | F | F | S | K152R; Q190S |
| 51 | R | D | A | F | N | Q | K152R; F156N |
| 52 | R | D | A | F | N | A | K152R; F156N; Q190A |
| 53 | R | D | A | F | N | S | K152R; F156N; Q190S |
| 54 | R | D | A | A | F | Q | K152R; F155A |
| 55 | R | D | A | A | F | A | K152R; F155A; Q190A |
| 56 | R | D | A | A | F | S | K152R; F155A; Q190S |
| 57 | R | D | A | A | N | Q | K152R; F155A; F156N |
| 58 | R | D | A | A | N | A | K152R; F155A; F156N; Q190A |
| 59 | R | D | A | A | N | S | K152R; F155A; F156N; Q190S |
| 60 | R | D | Y | F | F | Q | K152R; A154Y |
| 61 | R | D | Y | F | F | A | K152R; A154Y; Q190A |
| 62 | R | D | Y | F | F | S | K152R; A154Y; Q190S |
| 63 | R | D | Y | F | N | Q | K152R; A154Y; F156N |
| 64 | R | D | Y | F | N | A | K152R; A154Y; F156N; Q190A |
| 65 | R | D | Y | F | N | S | K152R; A154Y; F156N; Q190S |
| 66 | R | D | Y | A | F | Q | K152R; A154Y; F155A |
| 67 | R | D | Y | A | F | A | K152R; A154Y; F155A; Q190A |
| 68 | R | D | Y | A | F | S | K152R; A154Y; F155A; Q190S |
| 69 | R | D | Y | A | N | Q | K152R; A154Y; F155A; F156N |
| 70 | R | D | Y | A | N | A | K152R; A154Y; F155A; F156N; Q190A |
| 71 | R | D | Y | A | N | S | K152R; A154Y; F155A; F156N; Q190S |
| 72 | R | Y | A | F | F | Q | K152R; D153Y |
| 73 | R | Y | A | F | F | A | K152R; D153Y; Q190A |
| 74 | R | Y | A | F | F | S | K152R; D153Y; Q190S |
| 75 | R | Y | A | F | N | Q | K152R; D153Y; F156N |
| 76 | R | Y | A | F | N | A | K152R; D153Y; F156N; Q190A |
| 77 | R | Y | A | F | N | S | K152R; D153Y; F156N; Q190S |
| 78 | R | Y | A | A | F | Q | K152R; D153Y; F155A |
| 79 | R | Y | A | A | F | A | K152R; D153Y; F155A; Q190A |
| 80 | R | Y | A | A | F | S | K152R; D153Y; F155A; Q190S |
| 81 | R | Y | A | A | N | Q | K152R; D153Y; F155A; F156N |
| 82 | R | Y | A | A | N | A | K152R; D153Y; F155A; F156N; Q190A |
| 83 | R | Y | A | A | N | S | K152R; D153Y; F155A; F156N; Q190S |
| 84 | R | Y | Y | F | F | Q | K152R; D153Y; A154Y |
| 85 | R | Y | Y | F | F | A | K152R; D153Y; A154Y; Q190A |
| 86 | R | Y | Y | F | F | S | K152R; D153Y; A154Y; Q190S |
| 87 | R | Y | Y | F | N | Q | K152R; D153Y; A154Y; F156N |
| 88 | R | Y | Y | F | N | A | K152R; D153Y; A154Y; F156N; Q190A |
| 89 | R | Y | Y | F | N | S | K152R; D153Y; A154Y; F156N; Q190S |
| 90 | R | Y | Y | A | F | Q | K152R; D153Y; A154Y; F155A |
| 91 | R | Y | Y | A | F | A | K152R; D153Y; A154Y; F155A; Q190A |
| 92 | R | Y | Y | A | F | S | K152R; D153Y; A154Y; F155A; Q190S |
| 93 | R | Y | Y | A | N | Q | K152R; D153Y; A154Y; F155A; F156N |
| 94 | R | Y | Y | A | N | A | K152R; D153Y; A154Y; F155A; F156N; Q190A |
| 95 | R | Y | Y | A | N | S | K152R; D153Y; A154Y; F155A; F156N; Q190S |
| 96 | C | D | A | F | F | Q | K152C |
| 97 | C | D | A | F | F | A | K152C; Q190A |
| 98 | C | D | A | F | F | S | K152C; Q190S |
| 99 | C | D | A | F | N | Q | K152C; F156N |
| 100 | C | D | A | F | N | A | K152C; F156N; Q190A |
| 101 | C | D | A | F | N | S | K152C; F156N; Q190S |
| 102 | C | D | A | A | F | Q | K152C; F155A |
| 103 | C | D | A | A | F | A | K152C; F155A; Q190A |
| 104 | C | D | A | A | F | S | K152C; F155A; Q190S |
| 105 | C | D | A | A | N | Q | K152C; F155A; F156N |
| 106 | C | D | A | A | N | A | K152C; F155A; F156N; Q190A |
| 107 | C | D | A | A | N | S | K152C; F155A; F156N; Q190S |
| 108 | C | D | Y | F | F | Q | K152C; A154Y |
| 109 | C | D | Y | F | F | A | K152C; A154Y; Q190A |
| 110 | C | D | Y | F | F | S | K152C; A154Y; Q190S |
| 111 | C | D | Y | F | N | Q | K152C; A154Y; F156N |
| 112 | C | D | Y | F | N | A | K152C; A154Y; F156N; Q190A |
| 113 | C | D | Y | F | N | S | K152C; A154Y; F156N; Q190S |
| 114 | C | D | Y | A | F | Q | K152C; A154Y; F155A |
| 115 | C | D | Y | A | F | A | K152C; A154Y; F155A; Q190A |
| 116 | C | D | Y | A | F | S | K152C; A154Y; F155A; Q190S |
| 117 | C | D | Y | A | N | Q | K152C; A154Y; F155A; F156N |
| 118 | C | D | Y | A | N | A | K152C; A154Y; F155A; F156N; Q190A |
| 119 | C | D | Y | A | N | S | K152C; A154Y; F155A; F156N; Q190S |
| 120 | C | Y | A | F | F | Q | K152C; D153Y |
| 121 | C | Y | A | F | F | A | K152C; D153Y; Q190A |
| 122 | C | Y | A | F | F | S | K152C; D153Y; Q190S |
| 123 | C | Y | A | F | N | Q | K152C; D153Y; F156N |
| 124 | C | Y | A | F | N | A | K152C; D153Y; F156N; Q190A |
| 125 | C | Y | A | F | N | S | K152C; D153Y; F156N; Q190S |
| 126 | C | Y | A | A | F | Q | K152C; D153Y; F155A |
| 127 | C | Y | A | A | F | A | K152C; D153Y; F155A; Q190A |
| 128 | C | Y | A | A | F | S | K152C; D153Y; F155A; Q190S |
| 129 | C | Y | A | A | N | Q | K152C; D153Y; F155A; F156N |
| 130 | C | Y | A | A | N | A | K152C; D153Y; F155A; F156N; Q190A |
| 131 | C | Y | A | A | N | S | K152C; D153Y; F155A; F156N; Q190S |
| 132 | C | Y | Y | F | F | Q | K152C; D153Y; A154Y |
| 133 | C | Y | Y | F | F | A | K152C; D153Y; A154Y; Q190A |
| 134 | C | Y | Y | F | F | S | K152C; D153Y; A154Y; Q190S |
| 135 | C | Y | Y | F | N | Q | K152C; D153Y; A154Y; F156N |
| 136 | C | Y | Y | F | N | A | K152C; D153Y; A154Y; F156N; Q190A |
| 137 | C | Y | Y | F | N | S | K152C; D153Y; A154Y; F156N; Q190S |
| 138 | C | Y | Y | A | F | Q | K152C; D153Y; A154Y; F155A |
| 139 | C | Y | Y | A | F | A | K152C; D153Y; A154Y; F155A; Q190A |
| 140 | C | Y | Y | A | F | S | K152C; D153Y; A154Y; F155A; Q190S |
| 141 | C | Y | Y | A | N | Q | K152C; D153Y; A154Y; F155A; F156N |
| 142 | C | Y | Y | A | N | A | K152C; D153Y; A154Y; F155A; F156N; Q190A |
| 143 | C | Y | Y | A | N | S | K152C; D153Y; A154Y; F155A; F156N; Q190S |
| 144 | S | D | A | F | F | Q | K152S |
| 145 | S | D | A | F | F | A | K152S; Q190A |
| 146 | S | D | A | F | F | S | K152S; Q190S |
| 147 | S | D | A | F | N | Q | K152S; F156N |
| 148 | S | D | A | F | N | A | K152S; F156N; Q190A |
| 149 | S | D | A | F | N | S | K152S; F156N; Q190S |
| 150 | S | D | A | A | F | Q | K152S; F155A |
| 151 | S | D | A | A | F | A | K152S; F155A; Q190A |
| 152 | S | D | A | A | F | S | K152S; F155A; Q190S |
| 153 | S | D | A | A | N | Q | K152S; F155A; F156N |
| 154 | S | D | A | A | N | A | K152S; F155A; F156N; Q190A |
| 155 | S | D | A | A | N | S | K152S; F155A; F156N; Q190S |
| 156 | S | D | Y | F | F | Q | K152S; A154Y |
| 157 | S | D | Y | F | F | A | K152S; A154Y; Q190A |
| 158 | S | D | Y | F | F | S | K152S; A154Y; Q190S |
| 159 | S | D | Y | F | N | Q | K152S; A154Y; F156N |
| 160 | S | D | Y | F | N | A | K152S; A154Y; F156N; Q190A |
| 161 | S | D | Y | F | N | S | K152S; A154Y; F156N; Q190S |
| 162 | S | D | Y | A | F | Q | K152S; A154Y; F155A |
| 163 | S | D | Y | A | F | A | K152S; A154Y; F155A; Q190A |
| 164 | S | D | Y | A | F | S | K152S; A154Y; F155A; Q190S |
| 165 | S | D | Y | A | N | Q | K152S; A154Y; F155A; F156N |
| 166 | S | D | Y | A | N | A | K152S; A154Y; F155A; F156N; Q190A |
| 167 | S | D | Y | A | N | S | K152S; A154Y; F155A; F156N; Q190S |
| 168 | S | Y | A | F | F | Q | K152S; D153Y |
| 169 | S | Y | A | F | F | A | K152S; D153Y; Q190A |
| 170 | S | Y | A | F | F | S | K152S; D153Y; Q190S |
| 171 | S | Y | A | F | N | Q | K152S; D153Y; F156N |
| 172 | S | Y | A | F | N | A | K152S; D153Y; F156N; Q190A |
| 173 | S | Y | A | F | N | S | K152S; D153Y; F156N; Q190S |
| 174 | S | Y | A | A | F | Q | K152S; D153Y; F155A |
| 175 | S | Y | A | A | F | A | K152S; D153Y; F155A; Q190A |
| 176 | S | Y | A | A | F | S | K152S; D153Y; F155A; Q190S |
| 177 | S | Y | A | A | N | Q | K152S; D153Y; F155A; F156N |
| 178 | S | Y | A | A | N | A | K152S; D153Y; F155A; F156N; Q190A |
| 179 | S | Y | A | A | N | S | K152S; D153Y; F155A; F156N; Q190S |
| 180 | S | Y | Y | F | F | Q | K152S; D153Y; A154Y |
| 181 | S | Y | Y | F | F | A | K152S; D153Y; A154Y; Q190A |
| 182 | S | Y | Y | F | F | S | K152S; D153Y; A154Y; Q190S |
| 183 | S | Y | Y | F | N | Q | K152S; D153Y; A154Y; F156N |
| 184 | S | Y | Y | F | N | A | K152S; D153Y; A154Y; F156N; Q190A |
| 185 | S | Y | Y | F | N | S | K152S; D153Y; A154Y; F156N; Q190S |
| 186 | S | Y | Y | A | F | Q | K152S; D153Y; A154Y; F155A |
| 187 | S | Y | Y | A | F | A | K152S; D153Y; A154Y; F155A; Q190A |
| 188 | S | Y | Y | A | F | S | K152S; D153Y; A154Y; F155A; Q190S |
| 189 | S | Y | Y | A | N | Q | K152S; D153Y; A154Y; F155A; F156N |
| 190 | S | Y | Y | A | N | A | K152S; D153Y; A154Y; F155A; F156N; Q190A |
| 191 | S | Y | Y | A | N | S | K152S; D153Y; A154Y; F155A; F156N; Q190S |
| 192 | T | D | A | F | F | Q | K152T |
| 193 | T | D | A | F | F | A | K152T; Q190A |
| 194 | T | D | A | F | F | S | K152T; Q190S |
| 195 | T | D | A | F | N | Q | K152T; F156N |
| 196 | T | D | A | F | N | A | K152T; F156N; Q190A |
| 197 | T | D | A | F | N | S | K152T; F156N; Q190S |
| 198 | T | D | A | A | F | Q | K152T; F155A |
| 199 | T | D | A | A | F | A | K152T; F155A; Q190A |
| 200 | T | D | A | A | F | S | K152T; F155A; Q190S |
| 201 | T | D | A | A | N | Q | K152T; F155A; F156N |
| 202 | T | D | A | A | N | A | K152T; F155A; F156N; Q190A |
| 203 | T | D | A | A | N | S | K152T; F155A; F156N; Q190S |
| 204 | T | D | Y | F | F | Q | K152T; A154Y |
| 205 | T | D | Y | F | F | A | K152T; A154Y; Q190A |
| 206 | T | D | Y | F | F | S | K152T; A154Y; Q190S |
| 207 | T | D | Y | F | N | Q | K152T; A154Y; F156N |
| 208 | T | D | Y | F | N | A | K152T; A154Y; F156N; Q190A |
| 209 | T | D | Y | F | N | S | K152T; A154Y; F156N; Q190S |
| 210 | T | D | Y | A | F | Q | K152T; A154Y; F155A |
| 211 | T | D | Y | A | F | A | K152T; A154Y; F155A; Q190A |
| 212 | T | D | Y | A | F | S | K152T; A154Y; F155A; Q190S |
| 213 | T | D | Y | A | N | Q | K152T; A154Y; F155A; F156N |
| 214 | T | D | Y | A | N | A | K152T; A154Y; F155A; F156N; Q190A |
| 215 | T | D | Y | A | N | S | K152T; A154Y; F155A; F156N; Q190S |
| 216 | T | Y | A | F | F | Q | K152T; D153Y |
| 217 | T | Y | A | F | F | A | K152T; D153Y; Q190A |
| 218 | T | Y | A | F | F | S | K152T; D153Y; Q190S |
| 219 | T | Y | A | F | N | Q | K152T; D153Y; F156N |
| 220 | T | Y | A | F | N | A | K152T; D153Y; F156N; Q190A |
| 221 | T | Y | A | F | N | S | K152T; D153Y; F156N; Q190S |
| 222 | T | Y | A | A | F | Q | K152T; D153Y; F155A |
| 223 | T | Y | A | A | F | A | K152T; D153Y; F155A; Q190A |
| 224 | T | Y | A | A | F | S | K152T; D153Y; F155A; Q190S |
| 225 | T | Y | A | A | N | Q | K152T; D153Y; F155A; F156N |
| 226 | T | Y | A | A | N | A | K152T; D153Y; F155A; F156N; Q190A |
| 227 | T | Y | A | A | N | S | K152T; D153Y; F155A; F156N; Q190S |
| 228 | T | Y | Y | F | F | Q | K152T; D153Y; A154Y |
| 229 | T | Y | Y | F | F | A | K152T; D153Y; A154Y; Q190A |
| 230 | T | Y | Y | F | F | S | K152T; D153Y; A154Y; Q190S |
| 231 | T | Y | Y | F | N | Q | K152T; D153Y; A154Y; F156N |
| 232 | T | Y | Y | F | N | A | K152T; D153Y; A154Y; F156N; Q190A |
| 233 | T | Y | Y | F | N | S | K152T; D153Y; A154Y; F156N; Q190S |
| 234 | T | Y | Y | A | F | Q | K152T; D153Y; A154Y; F155A |
| 235 | T | Y | Y | A | F | A | K152T; D153Y; A154Y; F155A; Q190A |
| 236 | T | Y | Y | A | F | S | K152T; D153Y; A154Y; F155A; Q190S |
| 237 | T | Y | Y | A | N | Q | K152T; D153Y; A154Y; F155A; F156N |
| 238 | T | Y | Y | A | N | A | K152T; D153Y; A154Y; F155A; F156N; Q190A |
| 239 | T | Y | Y | A | N | S | K152T; D153Y; A154Y; F155A; F156N; Q190S |
| 240 | L | D | A | F | F | Q | K152L |
| 241 | L | D | A | F | F | A | K152L; Q190A |
| 242 | L | D | A | F | F | S | K152L; Q190S |
| 243 | L | D | A | F | N | Q | K152L; F156N |
| 244 | L | D | A | F | N | A | K152L; F156N; Q190A |
| 245 | L | D | A | F | N | S | K152L; F156N; Q190S |
| 246 | L | D | A | A | F | Q | K152L; F155A |
| 247 | L | D | A | A | F | A | K152L; F155A; Q190A |
| 248 | L | D | A | A | F | S | K152L; F155A; Q190S |
| 249 | L | D | A | A | N | Q | K152L; F155A; F156N |
| 250 | L | D | A | A | N | A | K152L; F155A; F156N; Q190A |
| 251 | L | D | A | A | N | S | K152L; F155A; F156N; Q190S |
| 252 | L | D | Y | F | F | Q | K152L; A154Y |
| 253 | L | D | Y | F | F | A | K152L; A154Y; Q190A |
| 254 | L | D | Y | F | F | S | K152L; A154Y; Q190S |
| 255 | L | D | Y | F | N | Q | K152L; A154Y; F156N |
| 256 | L | D | Y | F | N | A | K152L; A154Y; F156N; Q190A |
| 257 | L | D | Y | F | N | S | K152L; A154Y; F156N; Q190S |
| 258 | L | D | Y | A | F | Q | K152L; A154Y; F155A |
| 259 | L | D | Y | A | F | A | K152L; A154Y; F155A; Q190A |
| 260 | L | D | Y | A | F | S | K152L; A154Y; F155A; Q190S |
| 261 | L | D | Y | A | N | Q | K152L; A154Y; F155A; F156N |
| 262 | L | D | Y | A | N | A | K152L; A154Y; F155A; F156N; Q190A |
| 263 | L | D | Y | A | N | S | K152L; A154Y; F155A; F156N; Q190S |
| 264 | L | Y | A | F | F | Q | K152L; D153Y |
| 265 | L | Y | A | F | F | A | K152L; D153Y; Q190A |
| 266 | L | Y | A | F | F | S | K152L; D153Y; Q190S |
| 267 | L | Y | A | F | N | Q | K152L; D153Y; F156N |
| 268 | L | Y | A | F | N | A | K152L; D153Y; F156N; Q190A |
| 269 | L | Y | A | F | N | S | K152L; D153Y; F156N; Q190S |
| 270 | L | Y | A | A | F | Q | K152L; D153Y; F155A |
| 271 | L | Y | A | A | F | A | K152L; D153Y; F155A; Q190A |
| 272 | L | Y | A | A | F | S | K152L; D153Y; F155A; Q190S |
| 273 | L | Y | A | A | N | Q | K152L; D153Y; F155A; F156N |
| 274 | L | Y | A | A | N | A | K152L; D153Y; F155A; F156N; Q190A |
| 275 | L | Y | A | A | N | S | K152L; D153Y; F155A; F156N; Q190S |
| 276 | L | Y | Y | F | F | Q | K152L; D153Y; A154Y |
| 277 | L | Y | Y | F | F | A | K152L; D153Y; A154Y; Q190A |
| 278 | L | Y | Y | F | F | S | K152L; D153Y; A154Y; Q190S |
| 279 | L | Y | Y | F | N | Q | K152L; D153Y; A154Y; F156N |
| 280 | L | Y | Y | F | N | A | K152L; D153Y; A154Y; F156N; Q190A |
| 281 | L | Y | Y | F | N | S | K152L; D153Y; A154Y; F156N; Q190S |
| 282 | L | Y | Y | A | F | Q | K152L; D153Y; A154Y; F155A |
| 283 | L | Y | Y | A | F | A | K152L; D153Y; A154Y; F155A; Q190A |
| 284 | L | Y | Y | A | F | S | K152L; D153Y; A154Y; F155A; Q190S |
| 285 | L | Y | Y | A | N | Q | K152L; D153Y; A154Y; F155A; F156N |
| 286 | L | Y | Y | A | N | A | K152L; D153Y; A154Y; F155A; F156N; Q190A |
| 287 | L | Y | Y | A | N | S | K152L; D153Y; A154Y; F155A; F156N; Q190S |

Provided herein are compositions and methods for the incorporation of modified nucleotides into a nucleic acid chain. Polymerases variously comprise DNA polymerases, RNA polymerases, template-independent polymerases, reverse transcriptases, or other enzymes capable of nucleotide extension. Reverse transcriptases are RNA/DNA-dependent DNA polymerases that have been shown to tolerate certain types of nucleotide modifications, such as modifications to the 3′ position of the sugar. This property makes reverse transcriptases a desirable protein engineering target to further enhance reversible terminator (removable chemical groups which prevent nucleic acid extension) incorporation for applications such as sequencing. Further provided herein are methods of sequencing employing mutant polymerases that incorporate modified nucleotides. Further, the use of engineered reverse transcriptases allows the incorporation of mutations that enhance the thermostability of the enzyme or the ability of the enzyme to function at higher temperatures. Thermostable reverse transcriptases may be useful in isothermal sequencing or elongation techniques. Isothermal techniques include SDA, LAMP, SMAP, ICAN, SMART. In these techniques, the elongation reaction proceeds at a constant temperature, for example using strand displacement reactions. Amplification can be completed in a single step, by incubating the mixture of samples, primers, DNA polymerase with strand displacement activity, and substrates at a constant temperature. This reduces the number of steps required, eliminating thermal ramping steps and reducing the total cycle time for each sequencing or elongation cycle, while simultaneously decreasing the reaction time required for each cycle. Reverse transcriptases which may be used according to the methods and compositions of the present disclosure include viral, bacterial, and eukaryotic reverse transcriptases and homologs and orthologs thereof. Exemplary reverse transcriptases include but are not limited to Murine Moloney Leukemia Virus (MMLV) reverse transcriptase and engineered and/or truncated variants thereof; xenotropic murine leukemia virus (XMLV) reverse transcriptase and engineered and/or truncated variants thereof; HIV-1 reverse transcriptase and engineered and/or truncated variants thereof; HIV Type M reverse transcriptase and engineered and/or truncated variants thereof; HIV Type O reverse transcriptase and engineered and/or truncated variants thereof; HIV-2 reverse transcriptase and engineered and/or truncated variants thereof; Feline immunodeficiency Virus (FIV) reverse transcriptase and engineered and/or truncated variants thereof, and Sunscript® reverse transcriptase and engineered and/or truncated variants thereof. Other reverse transcriptases and homologous or orthologous polymerases are known in the art and are expressly contemplated within this disclosure. Provided herein are compositions and methods comprising mutant polypeptides which have enhanced thermostability. In some case, such mutant polypeptides possess polymerase activity (e.g., mutant nucleic acid polymerase). Thermostability in some instances includes increased Tm, resistance to degradation, and/or the ability to maintain functional activity (e.g., incorporation of nucleotides) at elevated temperatures relative to a nearest wild-type enzyme, such as a wild-type enzyme comprising a nearest wild-type enzyme sequence. Mutant polymerases in some cases comprise Tm that are increased about 1, 2, 5, 10, 15, 20, 25, or about 30 degrees C. relative to a nearest wild-type enzyme. Mutant polypeptides in some instances comprise a Tm that are increased at least 1, 2, 5, 10, 15, 20, 25, or at least 30 degrees C. relative to a nearest wild-type enzyme. Mutant polymerases often comprise a Tm value that are increased at least 1-10, 5-15, 4-20, 2-10, 4-15, 20-30, 10-60, or 25-35 degrees C. relative to a nearest wild-type enzyme. Polymerase activity, in some instances, comprises k_{cat}, k_{cat}/Kₘ, or yields of incorporated nucleotides for a given time period. In some cases, mutant polymerases functioning at an elevated temperature maintain at least 99%, 98%, 95%, 90%, 85%, or at least 80% of the optimal activity of a nearest wild-type enzyme functioning at a lower temperature. For example, mutant polymerases functioning at about 37 degrees C. maintain at least 99%, 98%, 95%, 90%, 85%, or at least 80% of the optimal activity of a nearest wild-type enzyme. In some instances, mutant polymerases functioning at about 42 degrees C. maintain at least 99%, 98%, 95%, 90%, 85%, or at least 80% of the optimal activity of a nearest wild-type enzyme. In some instances, mutant polymerases functioning at about 55 degrees C. maintain at least 99%, 98%, 95%, 90%, 85%, or at least 80% of the optimal activity of a nearest wild-type enzyme. In some instances, mutant polymerases functioning at about 60 degrees C. maintain at least 99%, 98%, 95%, 90%, 85%, or at least 80% of the optimal activity of a nearest wild-type enzyme. In some instances, mutant polymerases functioning at least at 50 degrees C. maintain at least 99%, 98%, 95%, 90%, 85%, or at least 80% of the optimal activity of a nearest wild-type enzyme. In some instances, mutant polymerases functioning at least at 60 degrees C. maintain at least 99%, 98%, 95%, 90%, 85%, or at least 80% of the optimal activity of a nearest wild-type enzyme. In some instances, mutant polymerases functioning at 37-95 degrees C. maintain at least 99%, 98%, 95%, 90%, 85%, or at least 80% of the optimal activity of a nearest wild-type enzyme. In some instances, mutant polymerases functioning at 37-95, 37-60, 37-55, 37-42, 40-60, 50-80, 42-55, 55-60, 55-95, 60-95, or 40-80 degrees C. maintain at least 99%, 98%, 95%, 90%, 85%, or at least 80% of the optimal activity of a nearest wild-type enzyme. In some instances, mutant polymerases functioning at 42-95 degrees C. maintain at least 99%, 98%, 95%, 90%, 85%, or at least 80% of the optimal activity of a nearest wild-type enzyme. In some instances, mutant polymerases functioning at 40-80 degrees C. maintain at least 99%, 98%, 95%, 90%, 85%, or at least 80% of the optimal activity of a nearest wild-type enzyme. In some instances, mutant polymerases functioning at 37-55 degrees C. maintain at least 99%, 98%, 95%, 90%, 85%, or at least 80% of the optimal activity of a nearest wild-type enzyme. In some instances, mutant polymerases functioning at 50-95 degrees C. maintain at least 99%, 98%, 95%, 90%, 85%, or at least 80% of the optimal activity of a nearest wild-type enzyme. In some instances, Mutant polymerases functioning at 60-95 degrees C. maintain at least 99%, 98%, 95%, 90%, 85%, or at least 80% of the optimal activity of a nearest wild-type enzyme. In some cases a mutant polymerase has an increased k_{cat} relative to a nearest related wild-type sequence functioning at a temperature of at least 37 degrees C. In some cases a mutant polymerase has an increased k_{cat} relative to a nearest related wild-type sequence functioning at a temperature of at least 42 degrees C. In some cases a mutant polymerase has an increased kcat relative to a nearest related wild-type sequence functioning at a temperature of at least 55 degrees C. In some cases a mutant polymerase has an increased k_{cat} relative to a nearest related wild-type sequence functioning at a temperature of at least 60 degrees C. In some cases a mutant polymerase has an increased kcat relative to a nearest related wild-type sequence functioning at a temperature of at least 80 degrees C. In some cases a mutant polymerase has an increased k_{cat} relative to a nearest related wild-type sequence functioning at a temperature of at least 90 degrees C. In some cases a mutant polymerase has an increased kcat relative to a nearest related wild-type sequence functioning at a temperature of 37-95, 37-60, 37-55, 37-42, 40-60, 50-80, 42-55, 55-60, 55-95, 60-95, or 40-80 degrees C. In some cases a mutant polymerase has an increased kcat relative to a nearest related wild-type sequence functioning at a temperature of 37-55 degrees C. In some cases a mutant polymerase has an increased kcat relative to a nearest related wild-type sequence functioning at a temperature of 35-80 degrees C.

Provided herein are compositions and methods comprising mutant polypeptides relating to enzymes exhibiting increased incorporation of 3′ modified nucleotides. Mutations in the polymerases described herein variously comprise one or more changes to amino acid residues present in the polypeptide. Additions, substitutions, or deletions are all examples of mutations that are used to generate mutant polypeptides. Substitutions in some instances comprise the exchange of one amino acid for an alternative amino acid, and such alternative amino acids differ from the original amino acid with regard to size, shape, conformation, or chemical structure. Mutations in some instances are conservative or non-conservative. Conservative mutations comprise the substitution of an amino acid with an amino acid that possesses similar chemical properties. Additions often comprise the insertion of one or more amino acids at the N-terminal, C-terminal, or internal positions of the polypeptide. In some cases, additions comprise fusion polypeptides, wherein one or more additional polypeptides is connected to the polypeptide. Such additional polypeptides in some instances comprise domains with additional activity, or sequences with additional function (e.g., improve expression, aid purification, improve solubility, attach to a solid support, or other function). Often a polypeptide described herein comprises one or more non-amino acid groups. Fusion polypeptides optionally comprise an amino acid or other chemical linker that connects the one or more proteins. Any number of mutations is introduced into a polypeptide or portion of a polypeptide described herein, such as 1, 2, 3, 4, 5, 6, 7, 8, 9, 10, 20, 50, or more than 50 mutations.

In some cases, entire domains (portions of the polypeptide with a defined function) are added, deleted or substituted with domains from other polypeptides. Exemplary domains include DNA/RNA binding domains, nucleotide binding domains, nuclease domains, subcellular localization domains such as nuclear localization domains, or other domains. In some embodiments, the methods and compositions of the present disclosure comprise the attachment of a domain serving as a spacer or label, and/or providing for the attachment of a linker such as a SNAP tag, an avidin moiety, a streptavidin moiety, an epitope tag, a fluorescent protein, an affinity tag, a metal binding (i.e., a His6 or polyhistidine tag) or the like. In some instances, one or more mutations are present in a catalytic site or binding domain. For example, a polypeptide comprises a reverse transcriptase-like binding domain comprising SEQ ID NO:14 or a functional equivalent thereof. A domain in some cases comprises a DNA/RNA binding site, for example comprising residues at positions 63, 64, 97, 112-115, 118, 119, and 190 of SEQ ID NO: 14. Such sites are optionally found in analogous positions after alignment of other sequences to SEQ ID NO: 14. In other instances, a domain comprises an RNase (ribonuclease) domain comprising residues at SEQ ID NO: 15, or functional equivalent thereof. In some cases, a polypeptide comprises an active site. The active site of a polypeptide may comprise, for example, residues 149-154, 190, 191, 221, 223, 224, 168, and 169 of SEQ ID NO: 14. Such sites are often found at analogous positions in other domains (identified by aligning the two or more sequences for comparison), and polypeptides that comprise such domains are consistent with methods and compositions described herein.

As used herein, the term “surrounding” an amino acid residue or sequence position has its ordinary meaning in the art, including and incorporating modifications such as substitutions, deletions, insertions, or post-translational modifications at residues from 1, 2, 3, 4, 5, 6, 7, 8, 9, 10, 11, or 12 or more residues distant from the named residue, i.e., N-terminal or C-terminal from the named residue. In some contexts, a residue greater than 12 residues or sequence positions N or C terminal from the named residue can be considered “surrounding” a named residue based on the sequence or structural (i.e., 3-dimensional) context as would be understood by one of ordinary skill in the art.

It is understood that substitutions or modifications of the residues described herein also may incorporate or may include nonstandard amino acids as are known in the art, including but not limited to hydroxyproline, N-formylmethionine, selenomethionine, selenocysteine, phosphotyrosine, phosphohistidine, and the like. The mutations, modifications, truncations, substitutions and the like as described herein may be made by any method as is known in the art, particularly the art of molecular biology and/or protein engineering. Such methods may include site directed mutagenesis using mutagenic and/or partially degenerate primers, in vitro gene assembly, gene editing (such as by CRISPR or related methods) and the like. The mutant or engineered proteins described herein may additionally be expressed, isolated, and/or purified by any such means as is known in the art. Relevant methods are described in: Green, M. and Sambrook, J., Molecular Cloning: A Laboratory Manual (Fourth Edition) which is hereby incorporated by reference in its entirety and especially with respect to its disclosure of methods for modifying, transferring, and expressing, recombinant, modified, and engineered gene sequences as well as extracting, isolating, and/or purifying engineered proteins.

In some embodiments, the compositions and methods of the present disclosure comprise one or more mutations that may affect thermal stability of the enzyme. In some embodiments, said mutation or mutations may comprise one or more substitutions, deletions, or insertions at, or at a position or location surrounding, positions 47, 86, 95, 117, 178, 280, 291, 308, 432, 502, 581, and/or 585 of SEQ ID NO 19, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue E47 of SEQ ID NO: 19 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E47 with aspartic acid, lysine, glutamine, arginine, histidine, valine, or alanine. In some embodiments, said mutation or mutations may comprise substitution of residue E47 with proline or glycine. In some embodiments, said mutation or mutations may comprise the substitution E47K. In some embodiments, said mutation or mutations may comprise the deletion of residue E47.

In some embodiments, said mutation or mutations may comprise substitution of residue D86 of SEQ ID NO: 19 with any of the 19 other natural amino acids (i.e., W, I, M, P, F, G, A, V, L, H, E, R, K, N, Y, C, S, T, or Q) or with non-natural amino acids as are known to those of skill in the art. In some embodiments, said mutation or mutations may comprise substitution of residue D86 with glutamic acid, asparagine, glutamine, valine, alanine, serine, or threonine. In some embodiments, said mutation or mutations may comprise substitution of residue D86 with proline or glycine. In some embodiments, said mutation or mutations may comprise the substitution D86A. In some embodiments, said mutation or mutations may comprise the deletion of residue D86.

In some embodiments, said mutation or mutations may comprise substitution of residue E95 of SEQ ID NO: 19 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E95 with aspartic acid, asparagine, glutamine, valine, alanine, serine, or threonine. In some embodiments, said mutation or mutations may comprise substitution of residue E95 with proline or glycine. In some embodiments, said mutation or mutations may comprise the substitution E95A. In some embodiments, said mutation or mutations may comprise the deletion of residue E95.

In some embodiments, said mutation or mutations may comprise substitution of residue L117 of SEQ ID NO: 19 with any of the 19 other natural amino acids (i.e., W, I, M, P, F, G, A, V, H, E, R, K, D, N, Y, C, S, T, or Q) or with non-natural amino acids as are known to those of skill in the art. In some embodiments, said mutation or mutations may comprise substitution of residue L117 with proline, glycine, alanine, serine, valine, or threonine. In some embodiments, said mutation or mutations may comprise substitution of residue L117 with proline or glycine. In some embodiments, said mutation or mutations may comprise the substitution L117P. In some embodiments, said mutation or mutations may comprise the deletion of residue L117.

In some embodiments, said mutation or mutations may comprise substitution of residue D178 of SEQ ID NO: 19 with any of the 19 other natural amino acids (i.e., W, I, M, P, F, G, A, V, L, H, E, R, K, N, Y, C, S, T, or Q) or with non-natural amino acids as are known to those of skill in the art. In some embodiments, said mutation or mutations may comprise substitution of residue D178 with asparagine, glutamic acid, glutamine, serine, threonine, or valine. In some embodiments, said mutation or mutations may comprise substitution of residue D178 with proline or glycine. In some embodiments, said mutation or mutations may comprise the substitution D178N. In some embodiments, said mutation or mutations may comprise the deletion of residue D178.

In some embodiments, said mutation or mutations may comprise substitution of residue E280 of SEQ ID NO: 19 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E280 with aspartic acid, lysine, arginine, glutamine, histidine, valine, or alanine. In some embodiments, said mutation or mutations may comprise substitution of residue E280 with proline or glycine. In some embodiments, said mutation or mutations may comprise the substitution E280K. In some embodiments, said mutation or mutations may comprise the substitution E280R. In some embodiments, said mutation or mutations may comprise the deletion of residue E280.

In some embodiments, said mutation or mutations may comprise substitution of residue W291 of SEQ ID NO: 19 with any of the 19 other natural amino acids (i.e., I, M, P, F, G, A, V, L, H, E, R, K, D, N, Y, C, S, T, or Q) or with non-natural amino acids as are known to those of skill in the art. In some embodiments, said mutation or mutations may comprise substitution of residue W291 with phenylalanine, tyrosine, histidine, leucine, isoleucine, methionine, or valine. In some embodiments, said mutation or mutations may comprise substitution of residue W291 with proline or glycine. In some embodiments, said mutation or mutations may comprise the substitution W291F. In some embodiments, said mutation or mutations may comprise the deletion of residue W291.

In some embodiments, said mutation or mutations may comprise substitution of residue T308 of SEQ ID NO: 19 with any of the 19 other natural amino acids (i.e., W, I, M, P, F, G, A, V, L, H, E, R, K, D, N, Y, C, S, or Q) or with non-natural amino acids as are known to those of skill in the art. In some embodiments, said mutation or mutations may comprise substitution of residue T308 with proline, glycine, alanine, serine, valine, or threonine. In some embodiments, said mutation or mutations may comprise substitution of residue T308 with proline or glycine. In some embodiments, said mutation or mutations may comprise the substitution T308P. In some embodiments, said mutation or mutations may comprise the deletion of residue T308.

In some embodiments, said mutation or mutations may comprise substitution of residue N432 of SEQ ID NO: 19 with any of the 19 other natural amino acids (i.e., W, I, M, P, F, G, A, V, L, H, E, R, K, D, Y, C, S, T, or Q) or with non-natural amino acids as are known to those of skill in the art. In some embodiments, said mutation or mutations may comprise substitution of residue N432 with aspartic acid, lysine, arginine, glutamic acid, glutamine, histidine, valine, or alanine. In some embodiments, said mutation or mutations may comprise substitution of residue N432 with proline or glycine. In some embodiments, said mutation or mutations may comprise the substitution N432K. In some embodiments, said mutation or mutations may comprise the deletion of residue N432.

In some embodiments, said mutation or mutations may comprise substitution of residue D502 of SEQ ID NO: 19 with any of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said mutation or mutations may comprise substitution of residue D502 with glutamic acid, asparagine, glutamine, valine, alanine, serine, or threonine. In some embodiments, said mutation or mutations may comprise substitution of residue D502 with proline or glycine. In some embodiments, said mutation or mutations may comprise the substitution D502A. In some embodiments, said mutation or mutations may comprise the deletion of residue D502.

In some embodiments, said mutation or mutations may comprise substitution of residue L581 of SEQ ID NO: 19 with any of the 19 other natural amino acids (i.e., W, I, M, P, F, G, A, V, H, E, R, K, D, N, Y, C, S, T, or Q) or with non-natural amino acids as are known to those of skill in the art. In some embodiments, said mutation or mutations may comprise substitution of residue L581 with phenylalanine, tyrosine, histidine, tryptophan, isoleucine, methionine, or valine. In some embodiments, said mutation or mutations may comprise substitution of residue L581 with proline or glycine. In some embodiments, said mutation or mutations may comprise the substitution L581W. In some embodiments, said mutation or mutations may comprise the deletion of residue L581.

In some embodiments, said mutation or mutations may comprise substitution of residue E585 of SEQ ID NO: 19 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E585 with aspartic acid, lysine, arginine, glutamine, histidine, valine, or alanine. In some embodiments, said mutation or mutations may comprise substitution of residue E585 with proline or glycine. In some embodiments, said mutation or mutations may comprise the substitution E585K. In some embodiments, said mutation or mutations may comprise the deletion of residue E585 of SEQ ID NO: 19.

In some embodiments, the mutation or mutations according to the methods and compositions of the present disclosure may comprise any 1, 2, 3, 4, 5, 6, 7, 8, 9, 10, 11, or 12 of E47K, D86A, E95A, L117P, D178N, E280K, E280R, W291F, T308P, N432K, D502A, L581W, and/or E585K, of SEQ ID NO: 19 or any subset or combination thereof, or any homolog or ortholog thereof.

In some embodiments, said mutation or mutations may comprise one or more substitutions, deletions, or insertions at, or at a position or location surrounding, positions 42, 77, 78, 86, 108, 162, 264, 285, 291, 292, 408, and/or 409 of SEQ ID NO: 20, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue E42 of SEQ ID NO: 20 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E47 with aspartic acid, lysine, glutamine, arginine, histidine, valine, or alanine. In some embodiments, said mutation or mutations may comprise substitution of residue E47 with proline or glycine. In some embodiments, said mutation or mutations may comprise the substitution E47K. In some embodiments, said mutation or mutations may comprise the deletion of residue E47.

In some embodiments, said mutation or mutations may comprise substitution of residue D77 of SEQ ID NO: 20 with any of the 19 other natural amino acids (i.e., W, I, M, P, F, G, A, V, L, H, E, R, K, N, Y, C, S, T, or Q) or with non-natural amino acids as are known to those of skill in the art. In some embodiments, said mutation or mutations may comprise substitution of residue D77 with glutamic acid, asparagine, glutamine, valine, alanine, serine, or threonine. In some embodiments, said mutation or mutations may comprise substitution of residue D77 with proline or glycine. In some embodiments, said mutation or mutations may comprise the substitution D77A. In some embodiments, said mutation or mutations may comprise the deletion of residue D77.

In some embodiments, said mutation or mutations may comprise substitution of residue Y78 of SEQ ID NO: 20 with any of the 19 other natural amino acids (i.e., W, I, M, P, F, G, A, V, L, H, E, R, K, D, N, C, S, T, or Q) or with non-natural amino acids as are known to those of skill in the art. In some embodiments, said mutation or mutations may comprise substitution of residue Y78 with glutamic acid, asparagine, glutamine, valine, alanine, serine, or threonine. In some embodiments, said mutation or mutations may comprise substitution of residue Y78 with proline or glycine. In some embodiments, said mutation or mutations may comprise the substitution Y78A. In some embodiments, said mutation or mutations may comprise the deletion of residue Y78.

In some embodiments, said mutation or mutations may comprise insertion of one or more residues between residues D77 and Y78 of SEQ ID NO: 20 with any one or more of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of glutamic acid, asparagine, glutamine, valine, alanine, serine, and/or threonine. In some embodiments, said mutation or mutations may comprise insertion of proline or glycine. In some embodiments, said mutation or mutations may comprise insertion of an alanine between residues D77 and Y78.

In some embodiments, said mutation or mutations may comprise substitution of residue E86 of SEQ ID NO: 20 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E86 with aspartic acid, asparagine, glutamine, valine, alanine, serine, or threonine. In some embodiments, said mutation or mutations may comprise substitution of residue E86 with proline or glycine. In some embodiments, said mutation or mutations may comprise the substitution E86A. In some embodiments, said mutation or mutations may comprise the deletion of residue E86.

In some embodiments, said mutation or mutations may comprise substitution of residue L108 of SEQ ID NO: 20 with any of the 19 other natural amino acids (i.e., W, I, M, P, F, G, A, V, H, E, R, K, D, N, Y, C, S, T, or Q) or with non-natural amino acids as are known to those of skill in the art. In some embodiments, said mutation or mutations may comprise substitution of residue L108 with proline, glycine, alanine, serine, valine, or threonine. In some embodiments, said mutation or mutations may comprise substitution of residue L108 with proline or glycine. In some embodiments, said mutation or mutations may comprise the substitution L108P. In some embodiments, said mutation or mutations may comprise the deletion of residue L108.

In some embodiments, said mutation or mutations may comprise substitution of residue D162 of SEQ ID NO: 20 with any of the 19 other natural amino acids (i.e., W, I, M, P, F, G, A, V, L, H, E, R, K, N, Y, C, S, T, or Q) or with non-natural amino acids as are known to those of skill in the art. In some embodiments, said mutation or mutations may comprise substitution of residue D162 with asparagine, glutamic acid, glutamine, serine, threonine, or valine. In some embodiments, said mutation or mutations may comprise substitution of residue D162 with proline or glycine. In some embodiments, said mutation or mutations may comprise the substitution D162N. In some embodiments, said mutation or mutations may comprise the deletion of residue D162.

In some embodiments, said mutation or mutations may comprise substitution of residue E264 of SEQ ID NO: 20 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E264 with aspartic acid, lysine, arginine, glutamine, histidine, valine, or alanine. In some embodiments, said mutation or mutations may comprise substitution of residue E264 with proline or glycine. In some embodiments, said mutation or mutations may comprise the substitution E264K. In some embodiments, said mutation or mutations may comprise the substitution E264R. In some embodiments, said mutation or mutations may comprise the deletion of residue E264.

In some embodiments, said mutation or mutations may comprise substitution of residue W285 of SEQ ID NO: 20 with any of the 19 other natural amino acids (i.e., I, M, P, F, G, A, V, L, H, E, R, K, D, N, Y, C, S, T, or Q) or with non-natural amino acids as are known to those of skill in the art. In some embodiments, said mutation or mutations may comprise substitution of residue W285 with phenylalanine, tyrosine, histidine, leucine, isoleucine, methionine, or valine. In some embodiments, said mutation or mutations may comprise substitution of residue W285 with proline or glycine. In some embodiments, said mutation or mutations may comprise the substitution W285F. In some embodiments, said mutation or mutations may comprise the deletion of residue W285.

In some embodiments, said mutation or mutations may comprise substitution of residue K291 of SEQ ID NO: 20 with any of the 19 other natural amino acids (i.e., W, I, M, P, F, G, A, V, L, H, E, R, D, N, Y, C, S, T, or Q) or with non-natural amino acids as are known to those of skill in the art. In some embodiments, said mutation or mutations may comprise substitution of residue K291 with proline, glycine, alanine, serine, valine, or threonine. In some embodiments, said mutation or mutations may comprise substitution of residue K291 with proline or glycine. In some embodiments, said mutation or mutations may comprise the substitution K291P. In some embodiments, said mutation or mutations may comprise the deletion of residue K291.

In some embodiments, said mutation or mutations may comprise substitution of residue T292 of SEQ ID NO: 20 with any of the 19 other natural amino acids (i.e., W, I, M, P, F, G, A, V, L, H, E, R, K, D, N, Y, C, S, or Q) or with non-natural amino acids as are known to those of skill in the art. In some embodiments, said mutation or mutations may comprise substitution of residue T292 with proline, glycine, alanine, serine, valine, or threonine. In some embodiments, said mutation or mutations may comprise substitution of residue T292 with proline or glycine. In some embodiments, said mutation or mutations may comprise the substitution T292P. In some embodiments, said mutation or mutations may comprise the deletion of residue T292.

In some embodiments, said mutation or mutations may comprise insertion of one or more residues between residues K291 and T292 of SEQ ID NO: 20 with any one or more of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of proline, glycine, alanine, serine, valine, glutamic acid, aspartic acid, and/or threonine. In some embodiments, said mutation or mutations may comprise insertion of proline or glycine. In some embodiments, said mutation or mutations may comprise insertion of a proline between residues K291 and T292.

In some embodiments, said mutation or mutations may comprise substitution of residue P408 of SEQ ID NO: 20 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P408 with aspartic acid, lysine, arginine, glutamic acid, glutamine, histidine, valine, or alanine. In some embodiments, said mutation or mutations may comprise substitution of residue P408 with proline or glycine. In some embodiments, said mutation or mutations may comprise the substitution P408K. In some embodiments, said mutation or mutations may comprise the deletion of residue P408.

In some embodiments, said mutation or mutations may comprise substitution of residue A409 of SEQ ID NO: 20 with any of the 19 other natural amino acids (i.e., W, I, M, P, F, G, V, L, H, E, R, K, D, N, Y, C, S, T, or Q) or with non-natural amino acids as are known to those of skill in the art. In some embodiments, said mutation or mutations may comprise substitution of residue A409 with aspartic acid, lysine, arginine, glutamic acid, glutamine, histidine, valine, or alanine. In some embodiments, said mutation or mutations may comprise substitution of residue A409 with proline or glycine. In some embodiments, said mutation or mutations may comprise the substitution A409K. In some embodiments, said mutation or mutations may comprise the deletion of residue A409.

In some embodiments, said mutation or mutations may comprise insertion of one or more residues between residues P408 and A409 of SEQ ID NO: 20 with any one or more of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of aspartic acid, lysine, arginine, glutamic acid, glutamine, histidine, tryptophan, leucine, valine, and/or alanine. In some embodiments, said mutation or mutations may comprise insertion of proline or glycine. In some embodiments, said mutation or mutations may comprise insertion of a lysine between residues P408 and A409 of SEQ ID NO: 20.

In some embodiments, the mutation or mutations according to the methods and compositions of the present disclosure may comprise any 1, 2, 3, 4, 5, 6, 7, 8, 9, 10, 11, or 12 of E42K, D77A, Y78A, E86A, L108P, D162N, E264K, E264R, W285F, K291P, T292P, P408K, and/or P409K, of SEQ ID NO: 20 or any subset or combination thereof, or any homolog or ortholog thereof.

In some embodiments, said mutation or mutations may comprise one or more substitutions, deletions, or insertions at, or at a position or location surrounding, positions 47, 83, 84, 92, 114, 175, 277, 288, 324, 325, 421, and/or 422 of SEQ ID NO: 21, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue E47 of SEQ ID NO: 21 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E47 with aspartic acid, lysine, glutamine, arginine, histidine, valine, or alanine. In some embodiments, said mutation or mutations may comprise substitution of residue E47 with proline or glycine. In some embodiments, said mutation or mutations may comprise the substitution E47K. In some embodiments, said mutation or mutations may comprise the deletion of residue E47.

In some embodiments, said mutation or mutations may comprise substitution of residue D83 of SEQ ID NO: 21 with any of the 19 other natural amino acids (i.e., W, I, M, P, F, G, A, V, L, H, E, R, K, N, Y, C, S, T, or Q) or with non-natural amino acids as are known to those of skill in the art. In some embodiments, said mutation or mutations may comprise substitution of residue D83 with glutamic acid, asparagine, glutamine, valine, alanine, serine, or threonine. In some embodiments, said mutation or mutations may comprise substitution of residue D83 with proline or glycine. In some embodiments, said mutation or mutations may comprise the substitution D83A. In some embodiments, said mutation or mutations may comprise the deletion of residue D83.

In some embodiments, said mutation or mutations may comprise substitution of residue Y84 of SEQ ID NO: 21 with any of the 19 other natural amino acids (i.e., W, I, M, P, F, G, A, V, L, H, E, R, K, D, N, C, S, T, or Q) or with non-natural amino acids as are known to those of skill in the art. In some embodiments, said mutation or mutations may comprise substitution of residue Y84 with glutamic acid, asparagine, glutamine, valine, alanine, serine, or threonine. In some embodiments, said mutation or mutations may comprise substitution of residue Y84 with proline or glycine. In some embodiments, said mutation or mutations may comprise the substitution Y84A. In some embodiments, said mutation or mutations may comprise the deletion of residue Y84.

In some embodiments, said mutation or mutations may comprise insertion of one or more residues between residues D83 and Y84 of SEQ ID NO: 21 with any of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of glutamic acid, asparagine, glutamine, valine, alanine, serine, and/or threonine. In some embodiments, said mutation or mutations may comprise insertion of proline or glycine. In some embodiments, said mutation or mutations may comprise insertion of an alanine between residues D83 and Y84.

In some embodiments, said mutation or mutations may comprise substitution of residue E92 of SEQ ID NO: 21 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E92 with aspartic acid, asparagine, glutamine, valine, alanine, serine, or threonine. In some embodiments, said mutation or mutations may comprise substitution of residue E92 with proline or glycine. In some embodiments, said mutation or mutations may comprise the substitution E92A. In some embodiments, said mutation or mutations may comprise the deletion of residue E92.

In some embodiments, said mutation or mutations may comprise substitution of residue L114 of SEQ ID NO: 21 with any of the 19 other natural amino acids (i.e., W, I, M, P, F, G, A, V, H, E, R, K, D, N, Y, C, S, T, or Q) or with non-natural amino acids as are known to those of skill in the art. In some embodiments, said mutation or mutations may comprise substitution of residue L114 with proline, glycine, alanine, serine, valine, or threonine. In some embodiments, said mutation or mutations may comprise substitution of residue L114 with proline or glycine. In some embodiments, said mutation or mutations may comprise the substitution L114P. In some embodiments, said mutation or mutations may comprise the deletion of residue L114.

In some embodiments, said mutation or mutations may comprise substitution of residue D175 of SEQ ID NO: 21 with any of the 19 other natural amino acids (i.e., W, I, M, P, F, G, A, V, L, H, E, R, K, N, Y, C, S, T, or Q) or with non-natural amino acids as are known to those of skill in the art. In some embodiments, said mutation or mutations may comprise substitution of residue D175 with asparagine, glutamic acid, glutamine, serine, threonine, or valine. In some embodiments, said mutation or mutations may comprise substitution of residue D175 with proline or glycine. In some embodiments, said mutation or mutations may comprise the substitution D175N. In some embodiments, said mutation or mutations may comprise the deletion of residue D175.

In some embodiments, said mutation or mutations may comprise substitution of residue E277 of SEQ ID NO: 21 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E277 with aspartic acid, lysine, arginine, glutamine, histidine, valine, or alanine. In some embodiments, said mutation or mutations may comprise substitution of residue E277 with proline or glycine. In some embodiments, said mutation or mutations may comprise the substitution E277K. In some embodiments, said mutation or mutations may comprise the substitution E277R. In some embodiments, said mutation or mutations may comprise the deletion of residue E277.

In some embodiments, said mutation or mutations may comprise substitution of residue W288 of SEQ ID NO: 21 with any of the 19 other natural amino acids (i.e., I, M, P, F, G, A, V, L, H, E, R, K, D, N, Y, C, S, T, or Q) or with non-natural amino acids as are known to those of skill in the art. In some embodiments, said mutation or mutations may comprise substitution of residue W288 with phenylalanine, tyrosine, histidine, leucine, isoleucine, methionine, or valine. In some embodiments, said mutation or mutations may comprise substitution of residue W288 with proline or glycine. In some embodiments, said mutation or mutations may comprise the substitution W288F. In some embodiments, said mutation or mutations may comprise the deletion of residue W288.

In some embodiments, said mutation or mutations may comprise substitution of residue K324 of SEQ ID NO: 21 with any of the 19 other natural amino acids (i.e., W, I, M, P, F, G, A, V, L, H, E, R, D, N, Y, C, S, T, or Q) or with non-natural amino acids as are known to those of skill in the art. In some embodiments, said mutation or mutations may comprise substitution of residue K324 with proline, glycine, alanine, serine, valine, or threonine. In some embodiments, said mutation or mutations may comprise substitution of residue K324 with proline or glycine. In some embodiments, said mutation or mutations may comprise the substitution K324P. In some embodiments, said mutation or mutations may comprise the deletion of residue K324.

In some embodiments, said mutation or mutations may comprise substitution of residue T325 of SEQ ID NO: 21 with any of the 19 other natural amino acids (i.e., W, I, M, P, F, G, A, V, L, H, E, R, K, D, N, Y, C, S, or Q) or with non-natural amino acids as are known to those of skill in the art. In some embodiments, said mutation or mutations may comprise substitution of residue T325 with proline, glycine, alanine, serine, valine, or threonine. In some embodiments, said mutation or mutations may comprise substitution of residue T325 with proline or glycine. In some embodiments, said mutation or mutations may comprise the substitution T325P. In some embodiments, said mutation or mutations may comprise the deletion of residue T325.

In some embodiments, said mutation or mutations may comprise insertion of one or more residues between residues K324 and T325 of SEQ ID NO: 21 with any one or more of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of proline, glycine, alanine, serine, valine, glutamic acid, aspartic acid, and/or threonine. In some embodiments, said mutation or mutations may comprise insertion of proline or glycine. In some embodiments, said mutation or mutations may comprise insertion of a proline between residues K324 and T325.

In some embodiments, said mutation or mutations may comprise substitution of residue P421 of SEQ ID NO: 21 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P421 with aspartic acid, lysine, arginine, glutamic acid, glutamine, histidine, valine, or alanine. In some embodiments, said mutation or mutations may comprise substitution of residue P421 with glycine. In some embodiments, said mutation or mutations may comprise the substitution P421K. In some embodiments, said mutation or mutations may comprise the deletion of residue P421.

In some embodiments, said mutation or mutations may comprise substitution of residue A422 of SEQ ID NO: 21 with any of the 19 other natural amino acids (i.e., W, I, M, P, F, G, V, L, H, E, R, K, D, N, Y, C, S, T, or Q) or with non-natural amino acids as are known to those of skill in the art. In some embodiments, said mutation or mutations may comprise substitution of residue A422 with aspartic acid, lysine, arginine, glutamic acid, glutamine, histidine, valine, or alanine. In some embodiments, said mutation or mutations may comprise substitution of residue A422 with proline or glycine. In some embodiments, said mutation or mutations may comprise the substitution A422K. In some embodiments, said mutation or mutations may comprise the deletion of residue A422.

In some embodiments, said mutation or mutations may comprise insertion of one or more residues between residues P421 and A422 of SEQ ID NO: 21 with any one or more of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of aspartic acid, lysine, arginine, glutamic acid, glutamine, histidine, tryptophan, leucine, valine, and/or alanine. In some embodiments, said mutation or mutations may comprise insertion of proline or glycine. In some embodiments, said mutation or mutations may comprise insertion of a lysine between residues P421 and A422 of SEQ ID NO: 21.

In some embodiments, the mutation or mutations according to the methods and compositions of the present disclosure may comprise any 1, 2, 3, 4, 5, 6, 7, 8, 9, 10, 11, 12, or 13 of E47K, D83A, Y84A, E92A, L114P, D175N, E277K, E277R, W288F, K324P, T325P, P421K, and/or A422K, of SEQ ID NO: 21 or any subset or combination thereof, or any homolog or ortholog thereof.

In some embodiments, said mutation or mutations may comprise one or more substitutions, deletions, or insertions at, or at a position or location surrounding, positions 10, 50, 59, 78 139, 296, 390, 391, 418, 701, 702, 791, 866, 868, and/or 870 of SEQ ID NO: 22, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue E10 of SEQ ID NO: 22 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E10 with aspartic acid, lysine, glutamine, arginine, histidine, valine, or alanine. In some embodiments, said mutation or mutations may comprise substitution of residue E10 with proline or glycine. In some embodiments, said mutation or mutations may comprise the substitution E10K. In some embodiments, said mutation or mutations may comprise the deletion of residue E10.

In some embodiments, said mutation or mutations may comprise substitution of residue K50 of SEQ ID NO: 22 with any of the 19 other natural amino acids (i.e., W, I, M, P, F, G, A, V, L, H, E, R, D, N, Y, C, S, T, or Q) or with non-natural amino acids as are known to those of skill in the art. In some embodiments, said mutation or mutations may comprise substitution of residue K50 with glutamic acid, asparagine, glutamine, valine, alanine, serine, or threonine. In some embodiments, said mutation or mutations may comprise substitution of residue K50 with proline or glycine. In some embodiments, said mutation or mutations may comprise the substitution K50A. In some embodiments, said mutation or mutations may comprise the deletion of residue K50.

In some embodiments, said mutation or mutations may comprise substitution of residue E59 of SEQ ID NO: 22 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E59 with aspartic acid, asparagine, glutamine, valine, alanine, serine, or threonine. In some embodiments, said mutation or mutations may comprise substitution of residue E59 with proline or glycine. In some embodiments, said mutation or mutations may comprise the substitution E59A. In some embodiments, said mutation or mutations may comprise the deletion of residue E59.

In some embodiments, said mutation or mutations may comprise substitution of residue L78 of SEQ ID NO: 22 with any of the 19 other natural amino acids (i.e., W, I, M, P, F, G, A, V, H, E, R, K, D, N, Y, C, S, T, or Q) or with non-natural amino acids as are known to those of skill in the art. In some embodiments, said mutation or mutations may comprise substitution of residue L78 with proline, glycine, alanine, serine, valine, or threonine. In some embodiments, said mutation or mutations may comprise substitution of residue L78 with proline or glycine. In some embodiments, said mutation or mutations may comprise the substitution L78P. In some embodiments, said mutation or mutations may comprise the deletion of residue L78.

In some embodiments, said mutation or mutations may comprise substitution of residue Q139 of SEQ ID NO: 22 with any of the 19 other natural amino acids (i.e., W, I, M, P, F, G, A, V, L, H, E, R, K, N, Y, C, S, T, or D) or with non-natural amino acids as are known to those of skill in the art. In some embodiments, said mutation or mutations may comprise substitution of residue Q139 with asparagine, glutamic acid, glutamine, serine, threonine, or valine. In some embodiments, said mutation or mutations may comprise substitution of residue Q139 with proline or glycine. In some embodiments, said mutation or mutations may comprise the substitution Q139N. In some embodiments, said mutation or mutations may comprise the deletion of residue Q139.

In some embodiments, said mutation or mutations may comprise substitution of residue Y296 of SEQ ID NO: 22 with any of the 19 other natural amino acids (i.e., W, I, M, P, F, G, A, V, L, H, E, R, K, D, N, C, S, T, or Q) or with non-natural amino acids as are known to those of skill in the art. In some embodiments, said mutation or mutations may comprise substitution of residue Y296 with aspartic acid, lysine, arginine, glutamine, histidine, valine, or alanine. In some embodiments, said mutation or mutations may comprise substitution of residue Y296 with proline or glycine. In some embodiments, said mutation or mutations may comprise the substitution Y296K. In some embodiments, said mutation or mutations may comprise the substitution Y296R. In some embodiments, said mutation or mutations may comprise the deletion of residue Y296.

In some embodiments, said mutation or mutations may comprise substitution of residue P390 of SEQ ID NO: 22 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P390 with phenylalanine, tyrosine, histidine, leucine, isoleucine, methionine, or valine. In some embodiments, said mutation or mutations may comprise substitution of residue P390 with glycine. In some embodiments, said mutation or mutations may comprise the substitution P390F. In some embodiments, said mutation or mutations may comprise the deletion of residue P390.

In some embodiments, said mutation or mutations may comprise substitution of residue P391 of SEQ ID NO: 22 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P391 with phenylalanine, tyrosine, histidine, leucine, isoleucine, methionine, or valine. In some embodiments, said mutation or mutations may comprise substitution of residue P391 with glycine. In some embodiments, said mutation or mutations may comprise the substitution P391F. In some embodiments, said mutation or mutations may comprise the deletion of residue P391.

In some embodiments, said mutation or mutations may comprise insertion of one or more residues between residues P390 and P391 of SEQ ID NO: 22 with any one or more of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of tryptophan, phenylalanine, tyrosine, histidine, leucine, isoleucine, serine, asparagine, methionine, and/or valine. In some embodiments, said mutation or mutations may comprise insertion of proline or glycine. In some embodiments, said mutation or mutations may comprise insertion of a phenylalanine between residues P390 and P391. In some embodiments, said mutation or mutations may comprise insertion of a tryptophan between residues P390 and P391.

In some embodiments, said mutation or mutations may comprise substitution of residue A418 of SEQ ID NO: 22 with any of the 19 other natural amino acids (i.e., W, I, M, P, F, G, V, L, H, E, R, K, D, N, Y, C, S, T, or Q) or with non-natural amino acids as are known to those of skill in the art. In some embodiments, said mutation or mutations may comprise substitution of residue A418 with proline, glycine, alanine, serine, valine, or threonine. In some embodiments, said mutation or mutations may comprise substitution of residue A418 with proline or glycine. In some embodiments, said mutation or mutations may comprise the substitution A418P. In some embodiments, said mutation or mutations may comprise the deletion of residue A418.

In some embodiments, said mutation or mutations may comprise substitution of residue L701 of SEQ ID NO: 22 with any of the 19 other natural amino acids (i.e., W, I, M, P, F, G, A, V, H, E, R, K, D, N, Y, C, S, T, or Q) or with non-natural amino acids as are known to those of skill in the art. In some embodiments, said mutation or mutations may comprise substitution of residue L701 with aspartic acid, lysine, arginine, glutamic acid, glutamine, histidine, valine, or alanine. In some embodiments, said mutation or mutations may comprise substitution of residue L701 with proline or glycine. In some embodiments, said mutation or mutations may comprise the substitution L701K. In some embodiments, said mutation or mutations may comprise the deletion of residue L701.

In some embodiments, said mutation or mutations may comprise substitution of residue D702 of SEQ ID NO: 22 with any of the 19 other natural amino acids (i.e., W, I, M, P, F, G, A, V, H, E, R, K, L, N, Y, C, S, T, or Q) or with non-natural amino acids as are known to those of skill in the art. In some embodiments, said mutation or mutations may comprise substitution of residue D702 with lysine, arginine, glutamic acid, glutamine, histidine, valine, or alanine. In some embodiments, said mutation or mutations may comprise substitution of residue D702 with proline or glycine. In some embodiments, said mutation or mutations may comprise the substitution D702K. In some embodiments, said mutation or mutations may comprise the deletion of residue D702.

In some embodiments, said mutation or mutations may comprise insertion of one or more residues between residues L701 and D702 of SEQ ID NO: 22 with any one or more of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of tryptophan, phenylalanine, tyrosine, histidine, leucine, isoleucine, serine, asparagine, methionine, and/or valine. In some embodiments, said mutation or mutations may comprise insertion of proline or glycine. In some embodiments, said mutation or mutations may comprise insertion of a lysine between residues L701 and D702.

In some embodiments, said mutation or mutations may comprise substitution of residue N791 of SEQ ID NO: 22 with any of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said mutation or mutations may comprise substitution of residue N791 with glutamic acid, asparagine, glutamine, valine, alanine, serine, or threonine. In some embodiments, said mutation or mutations may comprise substitution of residue N791 with proline or glycine. In some embodiments, said mutation or mutations may comprise the substitution N791A. In some embodiments, said mutation or mutations may comprise the deletion of residue N791.

In some embodiments, said mutation or mutations may comprise substitution of residue K866 of SEQ ID NO: 22 with any of the 19 other natural amino acids (i.e., W, I, M, P, F, G, A, V, H, E, R, L, D, N, Y, C, S, T, or Q) or with non-natural amino acids as are known to those of skill in the art. In some embodiments, said mutation or mutations may comprise substitution of residue K866 with phenylalanine, tyrosine, histidine, tryptophan, isoleucine, methionine, or valine. In some embodiments, said mutation or mutations may comprise substitution of residue K866 with proline or glycine. In some embodiments, said mutation or mutations may comprise the substitution K866W. In some embodiments, said mutation or mutations may comprise the deletion of residue K866.

In some embodiments, said mutation or mutations may comprise substitution of residue E868 of SEQ ID NO: 22 with any of the 19 other natural amino acids (i.e., W, I, M, P, F, G, A, V, H, L, R, K, D, N, Y, C, S, T, or Q) or with non-natural amino acids as are known to those of skill in the art. In some embodiments, said mutation or mutations may comprise substitution of residue E868 with phenylalanine, tyrosine, histidine, tryptophan, isoleucine, methionine, or valine. In some embodiments, said mutation or mutations may comprise substitution of residue E868 with proline or glycine. In some embodiments, said mutation or mutations may comprise the substitution E868W. In some embodiments, said mutation or mutations may comprise the deletion of residue E868.

In some embodiments, said mutation or mutations may comprise substitution of residue T870 of SEQ ID NO: 22 with any of the 19 other natural amino acids (i.e., W, I, M, P, F, G, A, V, L, H, R, K, D, N, Y, C, S, E, or Q) or with non-natural amino acids as are known to those of skill in the art. In some embodiments, said mutation or mutations may comprise substitution of residue T870 with aspartic acid, lysine, arginine, glutamine, histidine, valine, or alanine. In some embodiments, said mutation or mutations may comprise substitution of residue T870 with proline or glycine. In some embodiments, said mutation or mutations may comprise the substitution T870K. In some embodiments, said mutation or mutations may comprise the deletion of residue T870 of SEQ ID NO: 22.

In some embodiments, the mutation or mutations according to the methods and compositions of the present disclosure may comprise any 1, 2, 3, 4, 5, 6, 7, 8, 9, 10, 11, 12, 13, 14, 15, or 16 of E10K, K50A, E59A, L87P, Q139N, Y296K, Y296R, P390F, P391F, A418P, L701K, D702K, N791A, K866W, E868W and/or T870K, of SEQ ID NO: 22 or any subset or combination thereof, or any homolog or ortholog thereof.

In some embodiments, said mutation or mutations may comprise one or more substitutions, deletions, or insertions at, or at a position or location surrounding, positions 27, 65, 66, 73, 94, 146, 303, 392, 419, 704, 705, 795, 868, and/or 872 of SEQ ID NO: 23, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue E27 of SEQ ID NO: 23 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E27 with aspartic acid, lysine, glutamine, arginine, histidine, valine, or alanine. In some embodiments, said mutation or mutations may comprise substitution of residue E27 with proline or glycine. In some embodiments, said mutation or mutations may comprise the substitution E27K. In some embodiments, said mutation or mutations may comprise the deletion of residue E27.

In some embodiments, said mutation or mutations may comprise substitution of residue D65 of SEQ ID NO: 23 with any of the 19 other natural amino acids (i.e., W, I, M, P, F, G, A, V, L, H, E, R, K, N, Y, C, S, T, or Q) or with non-natural amino acids as are known to those of skill in the art. In some embodiments, said mutation or mutations may comprise substitution of residue D65 with glutamic acid, asparagine, glutamine, valine, alanine, serine, or threonine. In some embodiments, said mutation or mutations may comprise substitution of residue D65 with proline or glycine. In some embodiments, said mutation or mutations may comprise the substitution D65A. In some embodiments, said mutation or mutations may comprise the deletion of residue D65.

In some embodiments, said mutation or mutations may comprise substitution of residue R66 of SEQ ID NO: 23 with any of the 19 other natural amino acids (i.e., W, I, M, P, F, G, A, V, L, H, E, K, D, N, Y, C, S, T, or Q) or with non-natural amino acids as are known to those of skill in the art. In some embodiments, said mutation or mutations may comprise substitution of residue R66 with glutamic acid, asparagine, glutamine, valine, alanine, serine, or threonine. In some embodiments, said mutation or mutations may comprise substitution of residue R66 with proline or glycine. In some embodiments, said mutation or mutations may comprise the substitution R66A. In some embodiments, said mutation or mutations may comprise the deletion of residue R66.

In some embodiments, said mutation or mutations may comprise insertion of one or more residues between residues D65 and R66 of SEQ ID NO: 23 with any one or more of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of lysine, arginine, tryptophan, glutamic acid, asparagine, glutamine, valine, alanine, serine, and/or threonine. In some embodiments, said mutation or mutations may comprise insertion of proline or glycine. In some embodiments, said mutation or mutations may comprise insertion of an alanine between residues D65 and R66.

In some embodiments, said mutation or mutations may comprise substitution of residue E73 of SEQ ID NO: 23 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E73 with aspartic acid, asparagine, glutamine, valine, alanine, serine, or threonine. In some embodiments, said mutation or mutations may comprise substitution of residue E73 with proline or glycine. In some embodiments, said mutation or mutations may comprise the substitution E73A. In some embodiments, said mutation or mutations may comprise the deletion of residue E73.

In some embodiments, said mutation or mutations may comprise substitution of residue L94 of SEQ ID NO: 23 with any of the 19 other natural amino acids (i.e., W, I, M, P, F, G, A, V, H, E, R, K, D, N, Y, C, S, T, or Q) or with non-natural amino acids as are known to those of skill in the art. In some embodiments, said mutation or mutations may comprise substitution of residue L94 with proline, glycine, alanine, serine, valine, or threonine. In some embodiments, said mutation or mutations may comprise substitution of residue L94 with proline or glycine. In some embodiments, said mutation or mutations may comprise the substitution L94P. In some embodiments, said mutation or mutations may comprise the deletion of residue L94.

In some embodiments, said mutation or mutations may comprise substitution of residue Q146 of SEQ ID NO: 23 with any of the 19 other natural amino acids (i.e., W, I, M, P, F, G, A, V, L, H, E, R, K, N, Y, C, S, T, or D) or with non-natural amino acids as are known to those of skill in the art. In some embodiments, said mutation or mutations may comprise substitution of residue Q146 with asparagine, glutamic acid, glutamine, serine, threonine, or valine. In some embodiments, said mutation or mutations may comprise substitution of residue Q146 with proline or glycine. In some embodiments, said mutation or mutations may comprise the substitution Q146N. In some embodiments, said mutation or mutations may comprise the deletion of residue Q146.

In some embodiments, said mutation or mutations may comprise substitution of residue Y303 of SEQ ID NO: 23 with any of the 19 other natural amino acids (i.e., W, I, M, P, F, G, A, V, L, H, R, K, D, N, E, C, S, T, or Q) or with non-natural amino acids as are known to those of skill in the art. In some embodiments, said mutation or mutations may comprise substitution of residue Y303 with aspartic acid, lysine, arginine, glutamine, histidine, valine, or alanine. In some embodiments, said mutation or mutations may comprise substitution of residue Y303 with proline or glycine. In some embodiments, said mutation or mutations may comprise the substitution Y303K. In some embodiments, said mutation or mutations may comprise the substitution Y303R. In some embodiments, said mutation or mutations may comprise the deletion of residue Y303.

In some embodiments, said mutation or mutations may comprise substitution of residue W392 of SEQ ID NO: 23 with any of the 19 other natural amino acids (i.e., I, M, P, F, G, A, V, L, H, E, R, K, D, N, Y, C, S, T, or Q) or with non-natural amino acids as are known to those of skill in the art. In some embodiments, said mutation or mutations may comprise substitution of residue W392 with phenylalanine, tyrosine, histidine, leucine, isoleucine, methionine, or valine. In some embodiments, said mutation or mutations may comprise substitution of residue W392 with proline or glycine. In some embodiments, said mutation or mutations may comprise the substitution W392F. In some embodiments, said mutation or mutations may comprise the deletion of residue W392.

In some embodiments, said mutation or mutations may comprise substitution of residue A419 of SEQ ID NO: 23 with any of the 19 other natural amino acids (i.e., W, I, M, P, F, G, T, V, L, H, E, R, K, D, N, Y, C, S, or Q) or with non-natural amino acids as are known to those of skill in the art. In some embodiments, said mutation or mutations may comprise substitution of residue A419 with proline, glycine, serine, valine, or threonine. In some embodiments, said mutation or mutations may comprise substitution of residue A419 with proline or glycine. In some embodiments, said mutation or mutations may comprise the substitution A419P. In some embodiments, said mutation or mutations may comprise the deletion of residue A419.

In some embodiments, said mutation or mutations may comprise substitution of residue V704 of SEQ ID NO: 23 with any of the 19 other natural amino acids (i.e., W, I, M, P, F, G, A, L, H, E, R, K, D, Y, C, S, T, or Q) or with non-natural amino acids as are known to those of skill in the art. In some embodiments, said mutation or mutations may comprise substitution of residue V704 with aspartic acid, lysine, arginine, glutamic acid, glutamine, histidine, valine, or alanine. In some embodiments, said mutation or mutations may comprise substitution of residue V704 with proline or glycine. In some embodiments, said mutation or mutations may comprise the substitution V704K. In some embodiments, said mutation or mutations may comprise the deletion of residue V704.

In some embodiments, said mutation or mutations may comprise substitution of residue 1705 of SEQ ID NO: 23 with any of the 19 other natural amino acids (i.e., W, I, M, P, F, G, A, V, L, H, E, R, K, D, Y, C, S, T, or Q) or with non-natural amino acids as are known to those of skill in the art. In some embodiments, said mutation or mutations may comprise substitution of residue 1705 with aspartic acid, lysine, arginine, glutamic acid, glutamine, histidine, valine, or alanine. In some embodiments, said mutation or mutations may comprise substitution of residue 1705 with proline or glycine. In some embodiments, said mutation or mutations may comprise the substitution 1705K. In some embodiments, said mutation or mutations may comprise the deletion of residue 1705.

In some embodiments, said mutation or mutations may comprise insertion of one or more residues between residues V704 and 1705 of SEQ ID NO: 23 with any one or more of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of tryptophan, leucine, serine, aspartic acid, lysine, arginine, glutamic acid, glutamine, histidine, valine, and/or alanine. In some embodiments, said mutation or mutations may comprise insertion of proline or glycine. In some embodiments, said mutation or mutations may comprise insertion of an alanine between residues V704 and I705.

In some embodiments, said mutation or mutations may comprise substitution of residue K795 of SEQ ID NO: 23 with any of the 19 other natural amino acids (i.e., W, I, M, P, F, G, A, V, L, H, E, R, D, D, N, Y, C, S, T, or Q) or with non-natural amino acids as are known to those of skill in the art. In some embodiments, said mutation or mutations may comprise substitution of residue K795 with glutamic acid, asparagine, glutamine, valine, alanine, serine, or threonine. In some embodiments, said mutation or mutations may comprise substitution of residue K795 with proline or glycine. In some embodiments, said mutation or mutations may comprise the substitution K795A. In some embodiments, said mutation or mutations may comprise the deletion of residue K795.

In some embodiments, said mutation or mutations may comprise substitution of residue T868 of SEQ ID NO: 23 with any of the 19 other natural amino acids (i.e., W, I, M, P, F, G, A, V, H, E, R, K, D, N, Y, C, S, L, or Q) or with non-natural amino acids as are known to those of skill in the art. In some embodiments, said mutation or mutations may comprise substitution of residue T868 with phenylalanine, tyrosine, histidine, tryptophan, isoleucine, methionine, or valine. In some embodiments, said mutation or mutations may comprise substitution of residue T868 with proline or glycine. In some embodiments, said mutation or mutations may comprise the substitution T868W. In some embodiments, said mutation or mutations may comprise the deletion of residue T868.

In some embodiments, said mutation or mutations may comprise substitution of residue E872 of SEQ ID NO: 23 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E872 with aspartic acid, lysine, arginine, glutamine, histidine, valine, or alanine. In some embodiments, said mutation or mutations may comprise substitution of residue E872 with proline or glycine. In some embodiments, said mutation or mutations may comprise the substitution E872K. In some embodiments, said mutation or mutations may comprise the deletion of residue E872 of SEQ ID NO: 23.

In some embodiments, the mutation or mutations according to the methods and compositions of the present disclosure may comprise any 1, 2, 3, 4, 5, 6, 7, 8, 9, 10, 11, 12, 13, 14, or 15 of E27K, D65A, R66A, E73A, L94P, Q146N, Y303K, Y303R, W392F, A419P, V704K, 1705K, K795A, T868W, and/or E872K of SEQ ID NO: 23 or any subset or combination thereof, or any homolog or ortholog thereof.

In some embodiments, said mutation or mutations may comprise one or more substitutions, deletions, or insertions at, or at a position or location surrounding, positions 29, 70, 79, 100, 161, 318, 410, 443, 437, and/or 478 of SEQ ID NO: 24, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue E29 of SEQ ID NO: 24 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E29 with aspartic acid, lysine, glutamine, arginine, histidine, valine, or alanine. In some embodiments, said mutation or mutations may comprise substitution of residue E29 with proline or glycine. In some embodiments, said mutation or mutations may comprise the substitution E29K. In some embodiments, said mutation or mutations may comprise the deletion of residue E29.

In some embodiments, said mutation or mutations may comprise substitution of residue K70 of SEQ ID NO: 24 with any of the 19 other natural amino acids (i.e., W, I, M, P, F, G, A, V, L, H, E, R, D, N, Y, C, S, T, or Q) or with non-natural amino acids as are known to those of skill in the art. In some embodiments, said mutation or mutations may comprise substitution of residue K70 with glutamic acid, asparagine, glutamine, valine, alanine, serine, or threonine. In some embodiments, said mutation or mutations may comprise substitution of residue K70 with proline or glycine. In some embodiments, said mutation or mutations may comprise the substitution K70A. In some embodiments, said mutation or mutations may comprise the deletion of residue K70.

In some embodiments, said mutation or mutations may comprise substitution of residue E79 of SEQ ID NO: 24 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E79 with aspartic acid, asparagine, glutamine, valine, alanine, serine, or threonine. In some embodiments, said mutation or mutations may comprise substitution of residue E79 with proline or glycine. In some embodiments, said mutation or mutations may comprise the substitution E79A. In some embodiments, said mutation or mutations may comprise the deletion of residue E79.

In some embodiments, said mutation or mutations may comprise substitution of residue L100 of SEQ ID NO: 24 with any of the 19 other natural amino acids (i.e., W, I, M, P, F, G, A, V, H, E, R, K, D, N, Y, C, S, T, or Q) or with non-natural amino acids as are known to those of skill in the art. In some embodiments, said mutation or mutations may comprise substitution of residue L100 with proline, glycine, alanine, serine, valine, or threonine. In some embodiments, said mutation or mutations may comprise substitution of residue L100 with proline or glycine. In some embodiments, said mutation or mutations may comprise the substitution L100P. In some embodiments, said mutation or mutations may comprise the deletion of residue L100.

In some embodiments, said mutation or mutations may comprise substitution of residue Q161 of SEQ ID NO: 24 with any of the 19 other natural amino acids (i.e., W, I, M, P, F, G, A, V, L, H, E, R, K, N, Y, C, S, T, or D) or with non-natural amino acids as are known to those of skill in the art. In some embodiments, said mutation or mutations may comprise substitution of residue Q161 with asparagine, glutamic acid, glutamine, serine, threonine, or valine. In some embodiments, said mutation or mutations may comprise substitution of residue Q161 with proline or glycine. In some embodiments, said mutation or mutations may comprise the substitution Q161N. In some embodiments, said mutation or mutations may comprise the deletion of residue Q161.

In some embodiments, said mutation or mutations may comprise substitution of residue Y318 of SEQ ID NO: 24 with any of the 19 other natural amino acids (i.e., W, I, M, P, F, G, A, V, L, H, R, K, D, N, E C, S, T, or Q) or with non-natural amino acids as are known to those of skill in the art. In some embodiments, said mutation or mutations may comprise substitution of residue Y318 with aspartic acid, lysine, arginine, glutamine, histidine, valine, or alanine. In some embodiments, said mutation or mutations may comprise substitution of residue Y318 with proline or glycine. In some embodiments, said mutation or mutations may comprise the substitution Y318K. In some embodiments, said mutation or mutations may comprise the substitution Y318R. In some embodiments, said mutation or mutations may comprise the deletion of residue Y318.

In some embodiments, said mutation or mutations may comprise substitution of residue W410 of SEQ ID NO: 24 with any of the 19 other natural amino acids (i.e., I, M, P, F, G, A, V, L, H, E, R, K, D, N, Y, C, S, T, or Q) or with non-natural amino acids as are known to those of skill in the art. In some embodiments, said mutation or mutations may comprise substitution of residue W410 with phenylalanine, tyrosine, histidine, leucine, isoleucine, methionine, or valine. In some embodiments, said mutation or mutations may comprise substitution of residue W410 with proline or glycine. In some embodiments, said mutation or mutations may comprise the substitution W410F. In some embodiments, said mutation or mutations may comprise the deletion of residue W410.

In some embodiments, said mutation or mutations may comprise substitution of residue A437 of SEQ ID NO: 24 with any of the 19 other natural amino acids (i.e., W, I, M, P, F, G, A, V, L, H, E, R, K, D, N, Y, C, S, or Q) or with non-natural amino acids as are known to those of skill in the art. In some embodiments, said mutation or mutations may comprise substitution of residue A437 with proline, glycine, alanine, serine, valine, or threonine. In some embodiments, said mutation or mutations may comprise substitution of residue A437 with proline or glycine. In some embodiments, said mutation or mutations may comprise the substitution A437P. In some embodiments, said mutation or mutations may comprise the deletion of residue A437.

In some embodiments, said mutation or mutations may comprise substitution of any of residues 355-361 of SEQ ID NO: 24 with any of the 19 other natural amino acids (i.e., W, I, M, P, F, G, A, V, L, H, E, R, K, D, Y, C, S, T, or Q) or with non-natural amino acids as are known to those of skill in the art.

In some embodiments, said mutation or mutations may comprise substitution of residue K65 of SEQ ID NO: 24 with any of the 19 other natural amino acids (i.e., W, I, M, P, F, G, A, V, L, H, E, R, D, N, Y, C, S, T, or Q) or with non-natural amino acids as are known to those of skill in the art. In some embodiments, said mutation or mutations may comprise substitution of residue K65 with aspartic acid, lysine, arginine, glutamine, histidine, valine, or alanine. In some embodiments, said mutation or mutations may comprise substitution of residue K65 with proline or glycine. In some embodiments, said mutation or mutations may comprise the substitution K65R. In some embodiments, said mutation or mutations may comprise the deletion of residue K65.

In some embodiments, said mutation or mutations may comprise substitution of residue V75 of SEQ ID NO: 24 with any of the 19 other natural amino acids (i.e., W, I, M, P, F, G, A, V, H, E, R, K, D, N, Y, C, S, T, or Q) or with non-natural amino acids as are known to those of skill in the art. In some embodiments, said mutation or mutations may comprise substitution of residue V75 with phenylalanine, tryptophan, tyrosine, histidine, leucine, isoleucine, methionine, or valine. In some embodiments, said mutation or mutations may comprise substitution of residue V75 with proline or glycine. In some embodiments, said mutation or mutations may comprise the substitution V75I. In some embodiments, said mutation or mutations may comprise the deletion of residue V75. In some embodiments, said mutation or mutations comprise the combination of K65R and V75I.

In some embodiments, said mutation or mutations may comprise substitution of residue D443 of SEQ ID NO: 24 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue D443 with aspartic acid, asparagine, glutamic acid, glutamine, serine, threonine, or valine. In some embodiments, said mutation or mutations may comprise substitution of residue D443 with proline or glycine. In some embodiments, said mutation or mutations may comprise the substitution D443N. In some embodiments, said mutation or mutations may comprise the deletion of residue D443.

In some embodiments, said mutation or mutations may comprise substitution of residue E478 of SEQ ID NO: 24 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E478 with aspartic acid, asparagine, glutamic acid, glutamine, serine, threonine, or valine. In some embodiments, said mutation or mutations may comprise substitution of residue E478 with proline or glycine. In some embodiments, said mutation or mutations may comprise the substitution E478Q. In some embodiments, said mutation or mutations may comprise the deletion of residue E478 of SEQ ID NO: 24.

In some embodiments, the mutation or mutations according to the methods and compositions of the present disclosure may comprise any 1, 2, 3, 4, 5, 6, 7, 8, or 9 of E29K, K70A, E79A, L100P, Q161N, Y318K, Y318R, W410F, A437P, D443N, and/or E478Q of SEQ ID NO: 24 or any subset or combination thereof, or any homolog or ortholog thereof.

In some embodiments, said mutation or mutations may comprise one or more substitutions, deletions, or insertions at, or at a position or location surrounding, positions 31, 72, 81, 102, 163, 320, 412, 439, 730, 731, 828, 829, 832, 834, 900, and/or 904 of SEQ ID NO: 25, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue E31 of SEQ ID NO: 25 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E31 with aspartic acid, lysine, glutamine, arginine, histidine, valine, or alanine. In some embodiments, said mutation or mutations may comprise substitution of residue E31 with proline or glycine. In some embodiments, said mutation or mutations may comprise the substitution E31K. In some embodiments, said mutation or mutations may comprise the deletion of residue E31.

In some embodiments, said mutation or mutations may comprise substitution of residue K72 of SEQ ID NO: 25 with any of the 19 other natural amino acids (i.e., W, I, M, P, F, G, A, V, L, H, E, R, D, N, Y, C, S, T, or Q) or with non-natural amino acids as are known to those of skill in the art. In some embodiments, said mutation or mutations may comprise substitution of residue K72 with glutamic acid, asparagine, glutamine, valine, alanine, serine, or threonine. In some embodiments, said mutation or mutations may comprise substitution of residue K72 with proline or glycine. In some embodiments, said mutation or mutations may comprise the substitution K72A. In some embodiments, said mutation or mutations may comprise the deletion of residue K72.

In some embodiments, said mutation or mutations may comprise substitution of residue E81 of SEQ ID NO: 25 with any of the 19 other natural amino acids (i.e., W, I, M, P, F, G, A, V, L, H, R, K, D, N, Y, C, S, T, or Q) or with non-natural amino acids as are known to those of skill in the art. In some embodiments, said mutation or mutations may comprise substitution of residue E81 with aspartic acid, asparagine, glutamine, valine, alanine, serine, or threonine. In some embodiments, said mutation or mutations may comprise substitution of residue E81 with proline or glycine. In some embodiments, said mutation or mutations may comprise the substitution E81A. In some embodiments, said mutation or mutations may comprise the deletion of residue E81.

In some embodiments, said mutation or mutations may comprise substitution of residue L102 of SEQ ID NO: 25 with any of the 19 other natural amino acids (i.e., W, I, M, P, F, G, A, V, H, E, R, K, D, N, Y, C, S, T, or Q) or with non-natural amino acids as are known to those of skill in the art. In some embodiments, said mutation or mutations may comprise substitution of residue L102 with proline, glycine, alanine, serine, valine, or threonine. In some embodiments, said mutation or mutations may comprise substitution of residue L102 with proline or glycine. In some embodiments, said mutation or mutations may comprise the substitution L102P. In some embodiments, said mutation or mutations may comprise the deletion of residue L102.

In some embodiments, said mutation or mutations may comprise substitution of residue Q163 of SEQ ID NO: 25 with any of the 19 other natural amino acids (i.e., W, I, M, P, F, G, A, V, L, H, E, R, K, N, Y, C, S, T, or D) or with non-natural amino acids as are known to those of skill in the art. In some embodiments, said mutation or mutations may comprise substitution of residue Q163 with asparagine, aspartic acid, glutamine, serine, threonine, or valine. In some embodiments, said mutation or mutations may comprise substitution of residue Q163 with proline or glycine. In some embodiments, said mutation or mutations may comprise the substitution Q163N. In some embodiments, said mutation or mutations may comprise the deletion of residue Q163.

In some embodiments, said mutation or mutations may comprise substitution of residue Y320 of SEQ ID NO: 25 with any of the 19 other natural amino acids (i.e., W, I, M, P, F, G, A, V, L, H, R, K, D, N, E, C, S, T, or Q) or with non-natural amino acids as are known to those of skill in the art. In some embodiments, said mutation or mutations may comprise substitution of residue Y320 with aspartic acid, lysine, arginine, glutamine, histidine, valine, or alanine. In some embodiments, said mutation or mutations may comprise substitution of residue Y320 with proline or glycine. In some embodiments, said mutation or mutations may comprise the substitution Y320K. In some embodiments, said mutation or mutations may comprise the substitution Y320R. In some embodiments, said mutation or mutations may comprise the deletion of residue Y320.

In some embodiments, said mutation or mutations may comprise substitution of residue W412 of SEQ ID NO: 25 with any of the 19 other natural amino acids (i.e., I, M, P, F, G, A, V, L, H, E, R, K, D, N, Y, C, S, T, or Q) or with non-natural amino acids as are known to those of skill in the art. In some embodiments, said mutation or mutations may comprise substitution of residue W412 with phenylalanine, tyrosine, histidine, leucine, isoleucine, methionine, or valine. In some embodiments, said mutation or mutations may comprise substitution of residue W412 with proline or glycine. In some embodiments, said mutation or mutations may comprise the substitution W412F. In some embodiments, said mutation or mutations may comprise the deletion of residue W412.

In some embodiments, said mutation or mutations may comprise substitution of residue A439 of SEQ ID NO: 25 with any of the 19 other natural amino acids (i.e., W, I, M, P, F, G, T, V, L, H, E, R, K, D, N, Y, C, S, or Q) or with non-natural amino acids as are known to those of skill in the art. In some embodiments, said mutation or mutations may comprise substitution of residue A439 with proline, glycine, alanine, serine, valine, or threonine. In some embodiments, said mutation or mutations may comprise substitution of residue A439 with proline or glycine. In some embodiments, said mutation or mutations may comprise the substitution A439P. In some embodiments, said mutation or mutations may comprise the deletion of residue A439.

In some embodiments, said mutation or mutations may comprise substitution of residue D730 of SEQ ID NO: 25 with any of the 19 other natural amino acids (i.e., W, I, M, P, F, G, A, V, L, H, E, R, K, N, Y, C, S, T, or Q) or with non-natural amino acids as are known to those of skill in the art. In some embodiments, said mutation or mutations may comprise substitution of residue D730 with aspartic acid, lysine, arginine, glutamic acid, glutamine, histidine, valine, or alanine. In some embodiments, said mutation or mutations may comprise substitution of residue D730 with proline or glycine. In some embodiments, said mutation or mutations may comprise the substitution D730K. In some embodiments, said mutation or mutations may comprise the deletion of residue D730.

In some embodiments, said mutation or mutations may comprise substitution of residue 1731 of SEQ ID NO: 25 with any of the 19 other natural amino acids (i.e., W, D, M, P, F, G, A, V, L, H, E, R, K, N, Y, C, S, T, or Q) or with non-natural amino acids as are known to those of skill in the art. In some embodiments, said mutation or mutations may comprise substitution of residue 1731 with aspartic acid, lysine, arginine, glutamic acid, glutamine, histidine, valine, or alanine. In some embodiments, said mutation or mutations may comprise substitution of residue 1731 with proline or glycine. In some embodiments, said mutation or mutations may comprise the substitution 1731K. In some embodiments, said mutation or mutations may comprise the deletion of residue 1731.

In some embodiments, said mutation or mutations may comprise insertion of one or more residues between residues D730 and 1731 of SEQ ID NO: 25 with any one or more of the 19 other natural amino acids (i.e., W, I, M, P, F, G, A, V, L, H, E, R, K, D, N, Y, C, S, T, or Q) or with non-natural amino acids as are known to those of skill in the art. In some embodiments, said insertion may comprise 1, 2, 3, 4, 5, 6, 7, 8, 9, 10, 11, or 12 residues. In some embodiments, said insertion comprises the insertion of more than 12 residues. In some embodiments, said mutation or mutations may comprise insertion of one or more of lysine, tryptophan, leucine, glutamic acid, asparagine, glutamine, valine, alanine, serine, and/or threonine. In some embodiments, said mutation or mutations may comprise insertion of proline or glycine. In some embodiments, said mutation or mutations may comprise insertion of a lysine between residues D730 and I731.

In some embodiments, said mutation or mutations may comprise substitution of residue T828 of SEQ ID NO: 25 with any of the 19 other natural amino acids (i.e., W, I, M, P, F, G, A, V, L, H, E, R, K, D, N, Y, C, S, or Q) or with non-natural amino acids as are known to those of skill in the art. In some embodiments, said mutation or mutations may comprise substitution of residue T828 with glutamic acid, asparagine, glutamine, valine, alanine, serine, or threonine. In some embodiments, said mutation or mutations may comprise substitution of residue T828 with proline or glycine. In some embodiments, said mutation or mutations may comprise the substitution T828A. In some embodiments, said mutation or mutations may comprise the deletion of residue T828.

In some embodiments, said mutation or mutations may comprise substitution of residue K829 of SEQ ID NO: 25 with any of the 19 other natural amino acids (i.e., W, I, M, P, F, G, A, V, L, H, E, R, T, D, N, Y, C, S, or Q) or with non-natural amino acids as are known to those of skill in the art. In some embodiments, said mutation or mutations may comprise substitution of residue K829 with glutamic acid, asparagine, glutamine, valine, alanine, serine, or threonine. In some embodiments, said mutation or mutations may comprise substitution of residue K829 with proline or glycine. In some embodiments, said mutation or mutations may comprise the substitution K829A. In some embodiments, said mutation or mutations may comprise the deletion of residue K829.

In some embodiments, said mutation or mutations may comprise substitution of residue T832 of SEQ ID NO: 25 with any of the 19 other natural amino acids (i.e., W, I, M, P, F, G, A, V, L, H, E, R, K, D, N, Y, C, S, or Q) or with non-natural amino acids as are known to those of skill in the art. In some embodiments, said mutation or mutations may comprise substitution of residue T832 with glutamic acid, asparagine, glutamine, valine, alanine, serine, or threonine. In some embodiments, said mutation or mutations may comprise substitution of residue T832 with proline or glycine. In some embodiments, said mutation or mutations may comprise the substitution T832A. In some embodiments, said mutation or mutations may comprise the deletion of residue T832.

In some embodiments, said mutation or mutations may comprise substitution of residue V834 of SEQ ID NO: 25 with any of the 19 other natural amino acids (i.e., W, I, M, P, F, G, A, T, L, H, E, R, K, D, N, Y, C, S, or Q) or with non-natural amino acids as are known to those of skill in the art. In some embodiments, said mutation or mutations may comprise substitution of residue V834 with glutamic acid, asparagine, glutamine, valine, alanine, serine, or threonine. In some embodiments, said mutation or mutations may comprise substitution of residue V834 with proline or glycine. In some embodiments, said mutation or mutations may comprise the substitution V834A. In some embodiments, said mutation or mutations may comprise the deletion of residue V834.

In some embodiments, said mutation or mutations may comprise substitution of residue A900 of SEQ ID NO: 25 with any of the 19 other natural amino acids (i.e., W, I, M, P, F, G, L, V, H, E, R, K, D, N, Y, C, S, T, or Q) or with non-natural amino acids as are known to those of skill in the art. In some embodiments, said mutation or mutations may comprise substitution of residue A900 with phenylalanine, tyrosine, histidine, tryptophan, isoleucine, methionine, or valine. In some embodiments, said mutation or mutations may comprise substitution of residue A900 with proline or glycine. In some embodiments, said mutation or mutations may comprise the substitution A900W. In some embodiments, said mutation or mutations may comprise the deletion of residue A900.

In some embodiments, said mutation or mutations may comprise substitution of residue D904 of SEQ ID NO: 25 with any of the 19 other natural amino acids (i.e., W, I, M, P, F, G, A, V, L, H, R, K, E, N, Y, C, S, T, or Q) or with non-natural amino acids as are known to those of skill in the art. In some embodiments, said mutation or mutations may comprise substitution of residue D904 with aspartic acid, lysine, arginine, glutamine, histidine, valine, or alanine. In some embodiments, said mutation or mutations may comprise substitution of residue D904 with proline or glycine. In some embodiments, said mutation or mutations may comprise the substitution D904K. In some embodiments, said mutation or mutations may comprise the deletion of residue D904 of SEQ ID NO:

In some embodiments, the mutation or mutations according to the methods and compositions of the present disclosure may comprise any 1, 2, 3, 4, 5, 6, 7, 8, 9, 10, 11, 12, 13, 14, 15, 16, or 17 of E31K, K72A, E81A, L102P, Q163N, Y320K, Y320R, W412F, A439P, P730K, 1731K, T828A, K829A, T832A, V832A, A900W, and/or D904K of SEQ ID NO: 25 or any subset or combination thereof, or any homolog or ortholog thereof.

In some embodiments, the compositions and methods of the present disclosure comprise one or more mutations that may affect the ability of the enzyme to bind, interact with, or catalyze reactions involving, modified substrate molecules as described elsewhere herein, especially 3′-modified nucleotides, such as 3′-O-methyl, 3′-O-azido, and/or 3′-O-azidomethyl modified nucleotides. Binding, interaction, or catalysis may be measured according to various parameters as are known in the art for the characterization of enzyme-substrate interactions and catalysis, e.g., improvements in Kcat, Km, Kd, Ka, or other such parameters. In some embodiments, said mutation or mutations may comprise one or more substitutions, deletions, or insertions at, or at a position or location surrounding, positions 94, 105, 108, 130, 131, 166, 168, 200, 236, and/or 247 of SEQ ID NO: 19, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue R94 of SEQ ID NO: 19 with any of the 19 other natural amino acids (i.e., W, I, M, P, F, G, A, V, L, H, E, K, D, N, Y, C, S, T, or Q) or with non-natural amino acids as are known to those of skill in the art. In some embodiments, said mutation or mutations may comprise substitution of residue R94 with glycine, alanine, valine, cysteine, methionine, serine, threonine, leucine, phenylalanine, tyrosine or histidine. In some embodiments, said mutation or mutations may comprise substitution of residue R94 with alanine, proline or glycine. In some embodiments, said mutation or mutations may comprise any of the substitutions R94G, R94A, R94V. R94C, R94S, or R94F. In some embodiments, said mutation or mutations may comprise the deletion of residue R94.

In some embodiments, said mutation or mutations may comprise substitution of residue P105 of SEQ ID NO: 19 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P105 with lysine, arginine, histidine, threonine, serine, glutamic acid, glutamine, aspartic acid, asparagine, alanine, valine, or glycine. In some embodiments, said mutation or mutations may comprise substitution of residue P105 with alanine or glycine. In some embodiments, said mutation or mutations may comprise any of the substitutions P105K, P105T, P105R, P105E, P105A, or P105G. In some embodiments, said mutation or mutations may comprise the deletion of residue P105.

In some embodiments, said mutation or mutations may comprise substitution of residue P108 of SEQ ID NO: 19 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P108 with lysine, arginine, histidine, threonine, serine, glutamic acid, glutamine, aspartic acid, asparagine, alanine, valine, or glycine. In some embodiments, said mutation or mutations may comprise substitution of residue P108 with alanine, proline or glycine. In some embodiments, said mutation or mutations may comprise any of the substitutions P108R, P108S, P108K, P108N, P108T, P108E, P108D, P108A, or P108G. In some embodiments, said mutation or mutations may comprise the deletion of residue P108.

In some embodiments, said mutation or mutations may comprise substitution of residue K130 of SEQ ID NO: 19 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue K130 with threonine, serine, isoleucine, glutamic acid, glutamine, aspartic acid, asparagine, alanine, valine, or glycine. In some embodiments, said mutation or mutations may comprise substitution of residue K130 with alanine, proline or glycine. In some embodiments, said mutation or mutations may comprise any of the substitutions K130S, K130T, K130I, K130G, K130A, or K130V. In some embodiments, said mutation or mutations may comprise the deletion of residue K130.

In some embodiments, said mutation or mutations may comprise substitution of residue D131 of SEQ ID NO: 19 with any of the 19 other natural amino acids (i.e., W, I, M, F, G, A, V, L, H, E, R, P, N, Y, C, S, T, or Q) or with non-natural amino acids as are known to those of skill in the art. In some embodiments, said mutation or mutations may comprise substitution of residue D131 with arginine, lysine, threonine, serine, isoleucine, glutamic acid, glutamine, \asparagine, alanine, methionine, cysteine, valine, or glycine. In some embodiments, said mutation or mutations may comprise substitution of residue D131 with alanine, proline or glycine. In some embodiments, said mutation or mutations may comprise any of the substitutions D131R, D131T, D131I, D131M, D131G, D131A, or D131V. In some embodiments, said mutation or mutations may comprise the deletion of residue D131. In some embodiments, said mutation or mutations may comprise the truncation of SEQ ID NO: 19 N-terminal to residue D131, i.e., said mutation or mutations may comprise an alternative start codon for translation of SEQ ID NO: 19.

In some embodiments, mutations are present at positions 130 and 131. Exemplary combinations of mutations may comprise any of substitutions K130S, K130T, K130I, K130G, K130A, or K130V with any of substitutions D131R, D131T, D131I, D131M, D131G, D131A, or D131V.

In some embodiments, said mutation or mutations may comprise substitution of residue L166 of SEQ ID NO: 19 with any of the 19 other natural amino acids (i.e., W, I, M, F, G, A, V, H, E, R, P, D, N, Y, C, S, T, or Q) or with non-natural amino acids as are known to those of skill in the art. In some embodiments, said mutation or mutations may comprise substitution of residue L166 with arginine, lysine, threonine, serine, tyrosine, tryptophan, phenylalanine, histidine, isoleucine, glutamic acid, glutamine, aspartic acid, asparagine, alanine, methionine, cysteine, valine, proline, or glycine. In some embodiments, said mutation or mutations may comprise substitution of residue L166 with alanine, proline or glycine. In some embodiments, said mutation or mutations may comprise any of the substitutions L166W, L166I, L166M, L166P, L166F, L166G, L166A, L166V, L166H, L166E, L166R, L166K, L166D, L166N, L166Y, L166C, L166S, L166T, or L166Q. In some embodiments, said mutation or mutations may comprise the deletion of residue L166. In some embodiments, said mutation or mutations may comprise the truncation of SEQ ID NO: 19 N-terminal to residue L166, i.e., said mutation or mutations may comprise an alternative start codon for translation of SEQ ID NO: 19. In some embodiments, said mutation or mutations may comprise the truncation of SEQ ID NO: 19 C-terminal to residue L165, i.e., said mutation or mutations may comprise a stop codon terminating translation of SEQ ID NO: 19.

In some embodiments, said mutation or mutations may comprise substitution of residue Q168 of SEQ ID NO: 19 with any of the 19 other natural amino acids (i.e., W, I, M, F, G, A, V, L, H, E, R, P, D, N, Y, C, S, or T) or with non-natural amino acids as are known to those of skill in the art. In some embodiments, said mutation or mutations may comprise substitution of residue Q168 with lysine, arginine, glutamic acid, aspartic acid, asparagine, valine, alanine, serine, glycine, or threonine. In some embodiments, said mutation or mutations may comprise substitution of residue Q168 with alanine, proline or glycine. In some embodiments, said mutation or mutations may comprise any of the substitutions Q168K, Q168T, Q168R, Q168E, Q168A, or Q168G. In some embodiments, said mutation or mutations may comprise the deletion of residue Q168.

In some embodiments, said mutation or mutations may comprise substitution of residue Y200 of SEQ ID NO: 19 with any of the 19 other natural amino acids (i.e., W, I, M, F, G, A, V, L, H, E, R, P, D, N, C, S, T, or Q) or with non-natural amino acids as are known to those of skill in the art. In some embodiments, said mutation or mutations may comprise substitution of residue Y200 with lysine, arginine, glutamic acid, aspartic acid, asparagine, glutamine, valine, alanine, serine, proline, glycine, or threonine. In some embodiments, said mutation or mutations may comprise substitution of residue Y200 with alanine, proline or glycine. In some embodiments, said mutation or mutations may comprise any of the substitutions Y200K, Y200T, Y200R, Y200Q, Y200P, Y200E, Y200A, or Y200G. In some embodiments, said mutation or mutations may comprise the deletion of residue Y200.

In some embodiments, said mutation or mutations may comprise substitution of residue K236 of SEQ ID NO: 19 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue K236 with arginine, glutamic acid, aspartic acid, asparagine, glutamine, valine, alanine, serine, proline, glycine, or threonine. In some embodiments, said mutation or mutations may comprise substitution of residue K236 with alanine, proline or glycine. In some embodiments, said mutation or mutations may comprise any of the substitutions K236E, K236A, K236G, K236Q, K236P, or K236R. In some embodiments, said mutation or mutations may comprise the deletion of residue K236.

In some embodiments, said mutation or mutations may comprise substitution of residue L247 of SEQ ID NO: 19 with any of the 19 other natural amino acids (i.e., W, I, M, F, G, A, V, H, E, R, P, D, N, Y, C, S, T, or Q) or with non-natural amino acids as are known to those of skill in the art. In some embodiments, said mutation or mutations may comprise substitution of residue L247 with lysine, arginine, glutamic acid, aspartic acid, asparagine, glutamine, valine, alanine, serine, proline, glycine, or threonine. In some embodiments, said mutation or mutations may comprise substitution of residue L247 with alanine, proline or glycine. In some embodiments, said mutation or mutations may comprise any of the substitutions L247S, L247R, L247T, L247P, L247G, or L247A. In some embodiments, said mutation or mutations may comprise the deletion of residue L247 of SEQ ID NO: 19.

In some embodiments, mutations are present at positions 130 and 131. Exemplary combinations of mutations may comprise any of substitutions K130S, K130T, K130I, K130G, K130A, or K130V with any of substitutions D131R, D131T, D131I, D131M, D131G, D131A, or D131V. In some embodiments, said mutations may comprise substitutions at positions K130, D131, and/or L166 of SEQ ID NO: 19 or any combination thereof. In some embodiments, said mutations may optionally comprise any of substitutions K130S, K130T, K130I, K130G, K130A, or K130V, optionally in combination with any of substitutions D131R, D131T, D131I, D131M, D131G, D131A, or D131V and/or any of substitutions L166W, L166I, L166M, L166P, L166F, L166G, L166A, L166V, L166H, L166E, L166R, L166K, L166D, L166N, L166Y, L166C, L166S, L166T, or L166Q. In some embodiments, said mutation or mutations may comprise a combination of mutations at positions 130 and 131 of SEQ ID NO: 19 with the truncation of SEQ ID NO: 19 C-terminal to residue L165, i.e., said mutation or mutations may further comprise a stop codon terminating translation of SEQ ID NO: 19.

In some embodiments, mutations are present at positions 105, 236, and/or 247 of SEQ ID NO: 19. In some embodiments, said mutations may optionally comprise any of substitutions P105K, P105T, P105R, P105E, P105A, or P105G, optionally in combination with any of substitutions K236E, K236A, K236G, K236Q, K236P, or K236R and/or any of substitutions L247S, L247R, L247T, L247P, L247G, or L247A of SEQ ID NO: 19. In some embodiments, said mutations may optionally comprise any of substitutions K236E, K236A, K236G, K236Q, K236P, or K236R, optionally in combination with any of substitutions P105K, P105T, P105R, P105E, P105A, or P105G and/or any of substitutions L247S, L247R, L247T, L247P, L247G, or L247A of SEQ ID NO: 19. In some embodiments, said mutations may optionally comprise any of substitutions L247S, L247R, L247T, L247P, L247G, or L247A, optionally in combination with any of substitutions K236E, K236A, K236G, K236Q, K236P, or K236R and/or any of substitutions P105K, P105T, P105R, P105E, P105A, or P105G of SEQ ID NO: 19.

In some embodiments, mutations are present at positions 94, 200, and/or 247 of SEQ ID NO: 19. In some embodiments, said mutations may optionally comprise any of substitutions R94G, R94A, R94V. R94C, R94S, or R94F, optionally in combination with any of substitutions Y200K, Y200T, Y200R, Y200Q, Y200P, Y200E, Y200A, or Y200G and/or any of substitutions L247S, L247R, L247T, L247P, L247G, or L247A of SEQ ID NO: 19. In some embodiments, said mutations may optionally comprise any of substitutions Y200K, Y200T, Y200R, Y200Q, Y200P, Y200E, Y200A, or Y200G, optionally in combination with any of substitutions R94G, R94A, R94V. R94C, R94S, or R94F, and/or any of substitutions L247S, L247R, L247T, L247P, L247G, or L247A of SEQ ID NO: 19. In some embodiments, said mutations may optionally comprise any of substitutions L247S, L247R, L247T, L247P, L247G, or L247A, optionally in combination with any of substitutions R94G, R94A, R94V. R94C, R94S, or R94F, and/or any of substitutions L247S, L247R, L247T, L247P, L247G, or L247A, of SEQ ID NO: 19.

In some embodiments, mutations are present at positions 94, 108, and/or 168 of SEQ ID NO: 19. In some embodiments, said mutations may optionally comprise any of substitutions R94G, R94A, R94V. R94C, R94S, or R94F, optionally in combination with any of substitutions P108R, P108S, P108K, P108N, P108T, P108E, P108D, P108A, or P108G and/or any of substitutions Q168K, Q168T, Q168R, Q168E, Q168A, or Q168G of SEQ ID NO: 19. In some embodiments, said mutations may optionally comprise any of substitutions P108R, P108S, P108K, P108N, P108T, P108E, P108D, P108A, or P108G, optionally in combination with any of substitutions R94G, R94A, R94V. R94C, R94S, or R94F and/or any of substitutions Q168K, Q168T, Q168R, Q168E, Q168A, or Q168G of SEQ ID NO: 19. In some embodiments, said mutations may optionally comprise any of substitutions Q168K, Q168T, Q168R, Q168E, Q168A, or Q168G, optionally in combination with any of substitutions P108R, P108S, P108K, P108N, P108T, P108E, P108D, P108A, or P108G and/or any of substitutions R94G, R94A, R94V. R94C, R94S, or R94F of SEQ ID NO: 19.

In some embodiments, mutations are present at positions 168, 200, and/or 236 of SEQ ID NO: 19. In some embodiments, said mutations may optionally comprise any of substitutions K236E, K236A, K236G, K236Q, K236P, or K236R, optionally in combination with any of substitutions Y200V, Y200C, Y200F, Y200L, Y200S, Y200P, Y200K, Y200T, Y200R, Y200Q, Y200P, Y200E, Y200A, or Y200G and/or any of substitutions Q168K, Q168T, Q168R, Q168E, Q168A, or Q168G of SEQ ID NO: 19. In some embodiments, said mutations may optionally comprise any of substitutions Y200V, Y200C, Y200F, Y200L, Y200S, Y200P, Y200K, Y200T, Y200R, Y200Q, Y200P, Y200E, Y200A, or Y200G, optionally in combination with any of substitutions K236E, K236A, K236G, K236Q, K236P, or K236R, and/or any of substitutions Q168K, Q168T, Q168R, Q168E, Q168A, or Q168G of SEQ ID NO: 19. In some embodiments, said mutations may optionally comprise any of substitutions Q168K, Q168T, Q168R, Q168E, Q168A, or Q168G, optionally in combination with any of substitutions Y200K, Y200T, Y200R, Y200Q, Y200P, Y200E, Y200A, or Y200G and/or any of substitutions K236E, K236A, K236G, K236Q, K236P, or K236R, of SEQ ID NO: 19.

In some embodiments, the mutation or mutations according to the methods and compositions of the present disclosure may comprise any 1, 2, 3, 4, 5, 6, 7, 8, 9, or 10 of R94G, R94A, R94V. R94C, R94S, R94F, P105K, P105T, P105R, P105E, P105A, P105G, P108R, P108S, P108K, P108N, P108T, P108E, P108D, P108A, P108G, K130S, K130T, K130I, K130G, K130A, K130V, D131R, D131T, D131I, D131M, D131G, D131A, D131V, L166W, L166I, L166M, L166P, L166F, L166G, L166A, L166V, L166H, L166E, L166R, L166K, L166D, L166N, L166Y, L166C, L166S, L166T, L166Q, Q168K, Q168T, Q168R, Q168E, Q168A, Q168G, Y200V, Y200C, Y200F, Y200L, Y200S, Y200P, Y200K, Y200T, Y200R, Y200Q, Y200P, Y200E, Y200A, Y200G, K236E, K236A, K236G, K236Q, K236P, K236R, L247S, L247R, L247T, L247P, L247G, or L247A of SEQ ID NO: 19 or any subset or combination thereof, or any homolog or ortholog thereof.

In some embodiments, said mutation or mutations may comprise one or more substitutions, deletions, or insertions at, or at a position or location surrounding, positions 85, 96, 99, 121, 122, 150, 152, 184, 220, and/or 231 of SEQ ID NO: 20, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue R85 of SEQ ID NO: 20 with any of the 19 other natural amino acids (i.e., W, I, M, P, F, G, A, V, L, H, E, K, D, N, Y, C, S, T, or Q) or with non-natural amino acids as are known to those of skill in the art. In some embodiments, said mutation or mutations may comprise substitution of residue R85 with glycine, alanine, lysine, arginine, valine, cysteine, methionine, serine, threonine, leucine, phenylalanine, tyrosine or histidine. In some embodiments, said mutation or mutations may comprise substitution of residue R85 with alanine, proline or glycine. In some embodiments, said mutation or mutations may comprise any of the substitutions R85G, R85A, R85V. R85C, R85S, or R85F. In some embodiments, said mutation or mutations may comprise the deletion of residue R85.

In some embodiments, said mutation or mutations may comprise substitution of residue P96 of SEQ ID NO: 20 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P96 with lysine, arginine, histidine, threonine, serine, glutamic acid, glutamine, aspartic acid, asparagine, alanine, valine, or glycine. In some embodiments, said mutation or mutations may comprise substitution of residue P96 with alanine or glycine. In some embodiments, said mutation or mutations may comprise any of the substitutions P96K, P96T, P96R, P96E, P96A, or P96G. In some embodiments, said mutation or mutations may comprise the deletion of residue P96.

In some embodiments, said mutation or mutations may comprise substitution of residue P99 of SEQ ID NO: 20 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P96 with lysine, arginine, histidine, threonine, serine, glutamic acid, glutamine, aspartic acid, asparagine, alanine, valine, or glycine. In some embodiments, said mutation or mutations may comprise substitution of residue P99 with alanine or glycine. In some embodiments, said mutation or mutations may comprise any of the substitutions P99R, P99S, P99K, P99N, P99T, P99E, P99D, P99A, or P99G. In some embodiments, said mutation or mutations may comprise the deletion of residue P99.

In some embodiments, said mutation or mutations may comprise substitution of residue K121 of SEQ ID NO: 20 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue K121 with threonine, serine, isoleucine, glutamic acid, glutamine, aspartic acid, asparagine, alanine, valine, or glycine. In some embodiments, said mutation or mutations may comprise substitution of residue K121 with alanine, proline or glycine. In some embodiments, said mutation or mutations may comprise any of the substitutions K121S, K121T, K121I, K121G, K121A, or K121V. In some embodiments, said mutation or mutations may comprise the deletion of residue K121.

In some embodiments, said mutation or mutations may comprise substitution of residue D122 of SEQ ID NO: 20 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue D122 with arginine, lysine, threonine, serine, isoleucine, glutamic acid, glutamine, asparagine, alanine, methionine, cysteine, valine, or glycine. In some embodiments, said mutation or mutations may comprise substitution of residue D122 with alanine, proline or glycine. In some embodiments, said mutation or mutations may comprise any of the substitutions D122R, D122T, D122I, D122M, D122G, D122A, or D122V. In some embodiments, said mutation or mutations may comprise the deletion of residue D122. In some embodiments, said mutation or mutations may comprise the truncation of SEQ ID NO: 20 N-terminal to residue D122, i.e., said mutation or mutations may comprise an alternative start codon for translation of SEQ ID NO: 20.

In some embodiments, mutations are present at positions 121 and 122. Exemplary combinations of mutations may comprise any of substitutions K121S, K121T, K121I, K121G, K121A, or K121V with any of substitutions D122R, D122T, D122I, D122M, D122G, D122A, or D122V.

In some embodiments, said mutation or mutations may comprise substitution of residue L150 of SEQ ID NO: 20 with any of the 19 other natural amino acids (i.e., W, I, M, F, G, A, V, H, E, R, P, D, N, Y, C, S, T, or Q) or with non-natural amino acids as are known to those of skill in the art. In some embodiments, said mutation or mutations may comprise substitution of residue L150 with arginine, lysine, threonine, serine, tyrosine, tryptophan, phenylalanine, histidine, isoleucine, glutamic acid, glutamine, aspartic acid, asparagine, alanine, methionine, cysteine, valine, proline, or glycine. In some embodiments, said mutation or mutations may comprise substitution of residue L150 with alanine, proline or glycine. In some embodiments, said mutation or mutations may comprise any of the substitutions L150W, L150I, L150M, L150P, L150F, L150G, L150A, L150V, L150H, L150E, L150R, L150K, L150D, L150N, L150Y, L150C, L150S, L150T, or L150Q. In some embodiments, said mutation or mutations may comprise the deletion of residue L150. In some embodiments, said mutation or mutations may comprise the truncation of SEQ ID NO: 20 N-terminal to residue L150, i.e., said mutation or mutations may comprise an alternative start codon for translation of SEQ ID NO: 20. In some embodiments, said mutation or mutations may comprise the truncation of SEQ ID NO: 20 C-terminal to residue L149, i.e., said mutation or mutations may comprise a stop codon terminating translation of SEQ ID NO: 20.

In some embodiments, said mutation or mutations may comprise substitution of residue Q152 of SEQ ID NO: 20 with any of the 19 other natural amino acids (i.e., W, I, M, F, G, A, V, L, H, E, R, P, D, N, Y, C, S, or T) or with non-natural amino acids as are known to those of skill in the art. In some embodiments, said mutation or mutations may comprise substitution of residue Q152 with lysine, arginine, glutamic acid, aspartic acid, asparagine, valine, alanine, serine, glycine, or threonine. In some embodiments, said mutation or mutations may comprise substitution of residue Q152 with alanine, proline or glycine. In some embodiments, said mutation or mutations may comprise any of the substitutions Q152K, Q152T, Q152R, Q152E, Q152A, or Q152G. In some embodiments, said mutation or mutations may comprise the deletion of residue Q152.

In some embodiments, said mutation or mutations may comprise substitution of residue Y184 of SEQ ID NO: 20 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue Y184 with lysine, arginine, glutamic acid, aspartic acid, asparagine, glutamine, valine, alanine, serine, proline, glycine, or threonine. In some embodiments, said mutation or mutations may comprise substitution of residue Y184 with alanine, proline or glycine. In some embodiments, said mutation or mutations may comprise any of the substitutions Y184K, Y184T, Y184R, Y184Q, Y184P, Y184E, Y184A, or Y184G. In some embodiments, said mutation or mutations may comprise the deletion of residue Y184.

In some embodiments, said mutation or mutations may comprise substitution of residue K220 of SEQ ID NO: 20 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue K220 with arginine, glutamic acid, aspartic acid, asparagine, glutamine, valine, alanine, serine, proline, glycine, or threonine. In some embodiments, said mutation or mutations may comprise substitution of residue K220 with alanine, proline or glycine. In some embodiments, said mutation or mutations may comprise any of the substitutions K220E, K220A, K220G, K220Q, K220P, or K220R. In some embodiments, said mutation or mutations may comprise the deletion of residue K220.

In some embodiments, said mutation or mutations may comprise substitution of residue L231 of SEQ ID NO: 20 with any of the 19 other natural amino acids (i.e., W, I, M, F, G, A, V, H, E, R, P, D, N, Y, C, S, T, or Q) or with non-natural amino acids as are known to those of skill in the art. In some embodiments, said mutation or mutations may comprise substitution of residue L231 with lysine, arginine, glutamic acid, aspartic acid, asparagine, glutamine, valine, alanine, serine, proline, glycine, or threonine. In some embodiments, said mutation or mutations may comprise substitution of residue L231 with alanine, proline or glycine. In some embodiments, said mutation or mutations may comprise any of the substitutions L231S, L231R, L231T, L231P, L231G, or L231A. In some embodiments, said mutation or mutations may comprise the deletion of residue L231 of SEQ ID NO: 20.

In some embodiments, mutations are present at positions 121 and 122. Exemplary combinations of mutations may comprise any of substitutions K121S, K121T, K121I, K121G, K121A, or K121V with any of substitutions D122R, D122T, D122I, D122M, D122G, D122A, or D122V. In some embodiments, said mutations may comprise substitutions at positions K121, D122, and/or L150 of SEQ ID NO: 20 or any combination thereof. In some embodiments, said mutations may optionally comprise any of substitutions K121S, K121T, K121I, K121G, K121A, or K121V, optionally in combination with any of substitutions D122R, D122T, D122I, D122M, D122G, D122A, or D122V and/or any of substitutions L150W, L150I, L150M, L150P, L150F, L150G, L150A, L150V, L150H, L150E, L150R, L150K, L150D, L150N, L150Y, L150C, L150S, L150T, or L150Q. In some embodiments, said mutation or mutations may comprise a combination of mutations at positions 121 and 122 of SEQ ID NO: 20 with the truncation of SEQ ID NO: 20 C-terminal to residue L165, i.e., said mutation or mutations may further comprise a stop codon terminating translation of SEQ ID NO: 20.

In some embodiments, mutations are present at positions 96, 220, and/or 231 of SEQ ID NO: 20. In some embodiments, said mutations may optionally comprise any of substitutions P96K, P96T, P96R, P96E, P96A, or P96G, optionally in combination with any of substitutions K220E, K220A, K220G, K220Q, K220P, or K220R and/or any of substitutions L231S, L231R, L231T, L231P, L231G, or L231A of SEQ ID NO: 20. In some embodiments, said mutations may optionally comprise any of substitutions K220E, K220A, K220G, K220Q, K220P, or K220R, optionally in combination with any of substitutions 105K, P96T, P96R, P96E, P96A, or P96G and/or any of substitutions L231S, L231R, L231T, L231P, L231G, or L231A of SEQ ID NO: 20. In some embodiments, said mutations may optionally comprise any of substitutions L231S, L231R, L231T, L231P, L231G, or L231A, optionally in combination with any of substitutions K220E, K220A, K220G, K220Q, K220P, or K220R and/or any of substitutions P96K, P96T, P96R, P96E, P96A, or P96G of SEQ ID NO: 20.

In some embodiments, mutations are present at positions 85, 184, and/or 231 of SEQ ID NO: 20. In some embodiments, said mutations may optionally comprise any of substitutions R85G, R85A, R85V. R85C, R85S, or R85F, optionally in combination with any of substitutions Y184K, Y184T, Y184R, Y184Q, Y184P, Y184E, Y184A, or Y184G and/or any of substitutions L231S, L231R, L231T, L231P, L231G, or L231A of SEQ ID NO: 20. In some embodiments, said mutations may optionally comprise any of substitutions Y184K, Y184T, Y184R, Y184Q, Y184P, Y184E, Y184A, or Y184G, optionally in combination with any of substitutions R85G, R85A, R85V. R85C, R85S, or R85F, and/or any of substitutions L231S, L231R, L231T, L231P, L231G, or L231A of SEQ ID NO: 20. In some embodiments, said mutations may optionally comprise any of substitutions L231S, L231R, L231T, L231P, L231G, or L231A, optionally in combination with any of substitutions R85G, R85A, R85V. R85C, R85S, or R85F, and/or any of substitutions L231S, L231R, L231T, L231P, L231G, or L231A, of SEQ ID NO: 20.

In some embodiments, mutations are present at positions 85, 99, and/or 152 of SEQ ID NO: 20. In some embodiments, said mutations may optionally comprise any of substitutions R85G, R85A, R85V. R85C, R85S, or R85F, optionally in combination with any of substitutions P99R, P99S, P99K, P99N, P99T, P99E, P99D, P99A, or P99G and/or any of substitutions Q152K, Q152T, Q152R, Q152E, Q152A, or Q152G of SEQ ID NO: 20. In some embodiments, said mutations may optionally comprise any of substitutions P99R, P99S, P99K, P99N, P99T, P99E, P99D, P99A, or P99G, optionally in combination with any of substitutions R85G, R85A, R85V. R85C, R85S, or R85F and/or any of substitutions Q152K, Q152T, Q152R, Q152E, Q152A, or Q152G of SEQ ID NO: 20. In some embodiments, said mutations may optionally comprise any of substitutions Q152K, Q152T, Q152R, Q152E, Q152A, or Q152G, optionally in combination with any of substitutions P99R, P99S, P99K, P99N, P99T, P99E, P99D, P99A, or P99G and/or any of substitutions R85G, R85A, R85V. R85C, R85S, or R85F of SEQ ID NO: 20.

In some embodiments, mutations are present at positions 152, 184, and/or 220 of SEQ ID NO: 20. In some embodiments, said mutations may optionally comprise any of substitutions K220E, K220A, K220G, K220Q, K220P, or K220R, optionally in combination with any of substitutions Y184K, Y184T, Y184R, Y184Q, Y184P, Y184E, Y184A, or Y184G and/or any of substitutions Q152K, Q152T, Q152R, Q152E, Q152A, or Q152G of SEQ ID NO: 20. In some embodiments, said mutations may optionally comprise any of substitutions Y184K, Y184T, Y184R, Y184Q, Y184P, Y184E, Y184A, or Y184G, optionally in combination with any of substitutions K220E, K220A, K220G, K220Q, K220P, or K220R, and/or any of substitutions Q152K, Q152T, Q152R, Q152E, Q152A, or Q152G of SEQ ID NO: 20. In some embodiments, said mutations may optionally comprise any of substitutions Q152K, Q152T, Q152R, Q152E, Q152A, or Q152G, optionally in combination with any of substitutions Y184K, Y184T, Y184R, Y184Q, Y184P, Y184E, Y184A, or Y184G and/or any of substitutions K220E, K220A, K220G, K220Q, K220P, or K220R, of SEQ ID NO: 20.

In some embodiments, the mutation or mutations according to the methods and compositions of the present disclosure may comprise any 1, 2, 3, 4, 5, 6, 7, 8, 9, or 10 of R85G, R85A, R85V. R85C, R85S, R85F, P96K, P96T, P96R, P96E, P96A, P96G, P99R, P99S, P99K, P99N, P99T, P99E, P99D, P99A, P99G, K121S, K121T, K121I, K121G, K121A, K121V, D122R, D122T, D122I, D122M, D122G, D122A, D122V, L150W, L150I, L150M, L150P, L150F, L150G, L150A, L150V, L150H, L150E, L150R, L150K, L150D, L150N, L150Y, L150C, L150S, L150T, L150Q, Q152K, Q152T, Q152R, Q152E, Q152A, Q152G, Y184K, Y184T, Y184R, Y184Q, Y184P, Y184E, Y184A, Y184G, K220E, K220A, K220G, K220Q, K220P, K220R, L231S, L231R, L231T, L231P, L231G, or L231A of SEQ ID NO: 20 or any subset or combination thereof, or any homolog or ortholog thereof.

In some embodiments, said mutation or mutations may comprise one or more substitutions, deletions, or insertions at, or at a position or location surrounding, positions 91, 102, 105, 127, 128, 163, 165, 197, 233, and/or 244 of SEQ ID NO: 21, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue R91 of SEQ ID NO: 21 with any of the 19 other natural amino acids (i.e., W, I, M, P, F, G, A, V, L, H, E, K, D, N, Y, C, S, T, or Q) or with non-natural amino acids as are known to those of skill in the art. In some embodiments, said mutation or mutations may comprise substitution of residue R91 with glycine, lysine, arginine, alanine, valine, cysteine, methionine, serine, threonine, leucine, phenylalanine, tyrosine or histidine. In some embodiments, said mutation or mutations may comprise substitution of residue R91 with alanine, proline or glycine. In some embodiments, said mutation or mutations may comprise any of the substitutions R91G, R91A, R91V. R91C, R91S, or R91F. In some embodiments, said mutation or mutations may comprise the deletion of residue R91.

In some embodiments, said mutation or mutations may comprise substitution of residue P102 of SEQ ID NO: 21 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P102 with lysine, arginine, histidine, threonine, serine, glutamic acid, glutamine, aspartic acid, asparagine, alanine, valine, or glycine. In some embodiments, said mutation or mutations may comprise substitution of residue P102 with alanine or glycine. In some embodiments, said mutation or mutations may comprise any of the substitutions P102K, P102T, P102R, P102E, P102A, or P102G. In some embodiments, said mutation or mutations may comprise the deletion of residue P102.

In some embodiments, said mutation or mutations may comprise substitution of residue P105 of SEQ ID NO: 2121 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P102 with lysine, arginine, histidine, threonine, serine, glutamic acid, glutamine, aspartic acid, asparagine, alanine, valine, or glycine. In some embodiments, said mutation or mutations may comprise substitution of residue P105 with alanine or glycine. In some embodiments, said mutation or mutations may comprise any of the substitutions P105R, P105S, P105K, P105N, P105T, P105E, P105D, P105A, or P105G. In some embodiments, said mutation or mutations may comprise the deletion of residue P105.

In some embodiments, said mutation or mutations may comprise substitution of residue K127 of SEQ ID NO: 2121 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue K127 with threonine, lysine, arginine, serine, isoleucine, glutamic acid, glutamine, aspartic acid, asparagine, alanine, valine, or glycine. In some embodiments, said mutation or mutations may comprise substitution of residue K127 with alanine, proline or glycine. In some embodiments, said mutation or mutations may comprise any of the substitutions K127S, K127T, K127I, K127G, K127A, or K127V. In some embodiments, said mutation or mutations may comprise the deletion of residue K127.

In some embodiments, said mutation or mutations may comprise substitution of residue D128 of SEQ ID NO: 2121 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue D128 with arginine, lysine, threonine, serine, isoleucine, glutamic acid, glutamine, asparagine, alanine, methionine, cysteine, valine, or glycine. In some embodiments, said mutation or mutations may comprise substitution of residue D128 with alanine, proline or glycine. In some embodiments, said mutation or mutations may comprise any of the substitutions D128R, D128T, D128I, D128M, D128G, D128A, or D128V. In some embodiments, said mutation or mutations may comprise the deletion of residue D128. In some embodiments, said mutation or mutations may comprise the truncation of SEQ ID NO: 2121 N-terminal to residue D128, i.e., said mutation or mutations may comprise an alternative start codon for translation of SEQ ID NO: 2121.

In some embodiments, mutations are present at positions 127 and 128. Exemplary combinations of mutations may comprise any of substitutions K127S, K127T, K127I, K127G, K127A, or K127V with any of substitutions D128R, D128T, D128I, D128M, D128G, D128A, or D128V.

In some embodiments, said mutation or mutations may comprise substitution of residue L163 of SEQ ID NO: 2121 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue L163 with arginine, lysine, threonine, serine, tyrosine, tryptophan, phenylalanine, histidine, isoleucine, glutamic acid, glutamine, aspartic acid, asparagine, alanine, methionine, cysteine, valine, proline, or glycine. In some embodiments, said mutation or mutations may comprise substitution of residue L163 with alanine, proline or glycine. In some embodiments, said mutation or mutations may comprise any of the substitutions L163W, L163I, L163M, L163P, L163F, L163G, L163A, L163V, L163H, L163E, L163R, L163K, L163D, L163N, L163Y, L163C, L163S, L163T, or L163Q. In some embodiments, said mutation or mutations may comprise the deletion of residue L163. In some embodiments, said mutation or mutations may comprise the truncation of SEQ ID NO: 2121 N-terminal to residue L163, i.e., said mutation or mutations may comprise an alternative start codon for translation of SEQ ID NO: 2121. In some embodiments, said mutation or mutations may comprise the truncation of SEQ ID NO: 21 C-terminal to residue L165, i.e., said mutation or mutations may comprise a stop codon terminating translation of SEQ ID NO: 21.

In some embodiments, said mutation or mutations may comprise substitution of residue Q165 of SEQ ID NO: 21 with any of the 19 other natural amino acids (i.e., W, I, M, F, G, A, V, L, H, E, R, P, D, N, Y, C, S, or T) or with non-natural amino acids as are known to those of skill in the art. In some embodiments, said mutation or mutations may comprise substitution of residue Q165 with lysine, arginine, glutamic acid, aspartic acid, asparagine, valine, alanine, serine, glycine, or threonine. In some embodiments, said mutation or mutations may comprise substitution of residue Q165 with alanine, proline or glycine. In some embodiments, said mutation or mutations may comprise any of the substitutions Q165K, Q165T, Q165R, Q165E, Q165A, or Q165G. In some embodiments, said mutation or mutations may comprise the deletion of residue Q165.

In some embodiments, said mutation or mutations may comprise substitution of residue Y197 of SEQ ID NO: 21 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue Y197 with lysine, arginine, glutamic acid, aspartic acid, asparagine, glutamine, valine, alanine, serine, proline, glycine, or threonine. In some embodiments, said mutation or mutations may comprise substitution of residue Y197 with alanine, proline or glycine. In some embodiments, said mutation or mutations may comprise any of the substitutions Y197K, Y197T, Y197R, Y197Q, Y197P, Y197E, Y197A, or Y197G. In some embodiments, said mutation or mutations may comprise the deletion of residue Y197.

In some embodiments, said mutation or mutations may comprise substitution of residue K233 of SEQ ID NO: 21 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue K233 with arginine, glutamic acid, aspartic acid, asparagine, glutamine, valine, alanine, serine, proline, glycine, or threonine. In some embodiments, said mutation or mutations may comprise substitution of residue K233 with alanine, proline or glycine. In some embodiments, said mutation or mutations may comprise any of the substitutions K233E, K233A, K233G, K233Q, K233P, or K233R. In some embodiments, said mutation or mutations may comprise the deletion of residue K233.

In some embodiments, said mutation or mutations may comprise substitution of residue L244 of SEQ ID NO: 21 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue L244 with lysine, arginine, glutamic acid, aspartic acid, asparagine, glutamine, valine, alanine, serine, proline, glycine, or threonine. In some embodiments, said mutation or mutations may comprise substitution of residue L244 with alanine, proline or glycine. In some embodiments, said mutation or mutations may comprise any of the substitutions L244S, L244R, L244T, L244P, L244G, or L244A. In some embodiments, said mutation or mutations may comprise the deletion of residue L244 of SEQ ID NO: 21.

In some embodiments, mutations are present at positions 127 and 128. Exemplary combinations of mutations may comprise any of substitutions K127S, K127T, K127I, K127G, K127A, or K127V with any of substitutions D128R, D128T, D128I, D128M, D128G, D128A, or D128V. In some embodiments, said mutations may comprise substitutions at positions K127, D128, and/or L163 of SEQ ID NO: 21 or any combination thereof. In some embodiments, said mutations may optionally comprise any of substitutions K127S, K127T, K127I, K127G, K127A, or K127V, optionally in combination with any of substitutions D128R, D128T, D128I, D128M, D128G, D128A, or D128V and/or any of substitutions L163W, L163I, L163M, L163P, L163F, L163G, L163A, L163V, L163H, L163E, L163R, L163K, L163D, L163N, L163Y, L163C, L163S, L163T, or L163Q. In some embodiments, said mutation or mutations may comprise a combination of mutations at positions 127 and 128 of SEQ ID NO: 21 with the truncation of SEQ ID NO: 21 C-terminal to residue L162, i.e., said mutation or mutations may further comprise a stop codon terminating translation of SEQ ID NO: 21.

In some embodiments, mutations are present at positions 102, 233, and/or 244 of SEQ ID NO: 21. In some embodiments, said mutations may optionally comprise any of substitutions P102K, P102T, P102R, P102E, P102A, or P102G, optionally in combination with any of substitutions K233E, K233A, K233G, K233Q, K233P, or K233R and/or any of substitutions L244S, L244R, L244T, L244P, L244G, or L244A of SEQ ID NO: 21. In some embodiments, said mutations may optionally comprise any of substitutions K233E, K233A, K233G, K233Q, K233P, or K233R, optionally in combination with any of substitutions 105K, P102T, P102R, P102E, P102A, or P102G and/or any of substitutions L244S, L244R, L244T, L244P, L244G, or L244A of SEQ ID NO: 21. In some embodiments, said mutations may optionally comprise any of substitutions L244S, L244R, L244T, L244P, L244G, or L244A, optionally in combination with any of substitutions K233E, K233A, K233G, K233Q, K233P, or K233R and/or any of substitutions P102K, P102T, P102R, P102E, P102A, or P102G of SEQ ID NO: 21.

In some embodiments, mutations are present at positions 91, 197, and/or 244 of SEQ ID NO: 21. In some embodiments, said mutations may optionally comprise any of substitutions R91G, R91A, R91V. R91C, R91S, or R91F, optionally in combination with any of substitutions Y197K, Y197T, Y197R, Y197Q, Y197P, Y197E, Y197A, or Y197G and/or any of substitutions L244S, L244R, L244T, L244P, L244G, or L244A of SEQ ID NO: 21. In some embodiments, said mutations may optionally comprise any of substitutions Y197K, Y197T, Y197R, Y197Q, Y197P, Y197E, Y197A, or Y197G, optionally in combination with any of substitutions R91G, R91A, R91V. R91C, R91S, or R91F, and/or any of substitutions L244S, L244R, L244T, L244P, L244G, or L244A of SEQ ID NO: 21. In some embodiments, said mutations may optionally comprise any of substitutions L244S, L244R, L244T, L244P, L244G, or L244A, optionally in combination with any of substitutions R91G,
R91A, R91V. R91C, R91S, or R91F, and/or any of substitutions L244S, L244R, L244T, L244P, L244G, or L244A, of SEQ ID NO: 21.

In some embodiments, mutations are present at positions 91, 105, and/or 165 of SEQ ID NO: 21. In some embodiments, said mutations may optionally comprise any of substitutions R91G, R91A, R91V. R91C, R91S, or R91F, optionally in combination with any of substitutions P105R, P105S, P105K, P105N, P105T, P105E, P105D, P105A, or P105G and/or any of substitutions Q165K, Q165T, Q165R, Q165E, Q165A, or Q165G of SEQ ID NO: 21. In some embodiments, said mutations may optionally comprise any of substitutions P105R, P105S, P105K, P105N, P105T, P105E, P105D, P105A, or P105G, optionally in combination with any of substitutions R91G, R91A, R91V. R91C, R91S, or R91F and/or any of substitutions Q165K, Q165T, Q165R, Q165E, Q165A, or Q165G of SEQ ID NO: 21. In some embodiments, said mutations may optionally comprise any of substitutions Q165K, Q165T, Q165R, Q165E, Q165A, or Q165G, optionally in combination with any of substitutions P105R, P105S, P105K, P105N, P105T, P105E, P105D, P105A, or P105G and/or any of substitutions R91G, R91A, R91V. R91C, R91S, or R91F of SEQ ID NO: 21.

In some embodiments, mutations are present at positions 165, 197, and/or 233 of SEQ ID NO: 21. In some embodiments, said mutations may optionally comprise any of substitutions K233E, K233A, K233G, K233Q, K233P, or K233R, optionally in combination with any of substitutions Y197K, Y197T, Y197R, Y197Q, Y197P, Y197E, Y197A, or Y197G and/or any of substitutions Q165K, Q165T, Q165R, Q165E, Q165A, or Q165G of SEQ ID NO: 21. In some embodiments, said mutations may optionally comprise any of substitutions Y197K, Y197T, Y197R, Y197Q, Y197P, Y197E, Y197A, or Y197G, optionally in combination with any of substitutions K233E, K233A, K233G, K233Q, K233P, or K233R, and/or any of substitutions Q165K, Q165T, Q165R, Q165E, Q165A, or Q165G of SEQ ID NO: 21. In some embodiments, said mutations may optionally comprise any of substitutions Q165K, Q165T, Q165R, Q165E, Q165A, or Q165G, optionally in combination with any of substitutions Y197K, Y197T, Y197R, Y197Q, Y197P, Y197E, Y197A, or Y197G and/or any of substitutions K233E, K233A, K233G, K233Q, K233P, or K233R, of SEQ ID NO: 21.

In some embodiments, the mutation or mutations according to the methods and compositions of the present disclosure may comprise any 1, 2, 3, 4, 5, 6, 7, 8, 9, or 10 of R91G, R91A, R91V. R91C, R91S, R91F, P102K, P102T, P102R, P102E, P102A, P102G, P105R, P105S, P105K, P105N, P105T, P105E, P105D, P105A, P105G, K127S, K127T, K127I, K127G, K127A, K127V, D128R, D128T, D128I, D128M, D128G, D128A, D128V, L163W, L163I, L163M, L163P, L163F, L163G, L163A, L163V, L163H, L163E, L163R, L163K, L163D, L163N, L163Y, L163C, L163S, L163T, L163Q, Q165K, Q165T, Q165R, Q165E, Q165A, Q165G, Y197K, Y197T, Y197R, Y197Q, Y197P, Y197E, Y197A, Y197G, K236E, K233E, K233A, K233G, K233Q, K233P, K233R, L244S, L244R, L244T, L244P, L244G, or L244A of SEQ ID NO: 21 or any subset or combination thereof, or any homolog or ortholog thereof.

In some embodiments, said mutation or mutations may comprise one or more substitutions, deletions, or insertions at, or at a position or location surrounding, positions 58, 70, 73, 90, 91, 127, 129, 161, 198, and/or 208 of SEQ ID NO: 22, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue R58 of SEQ ID NO: 22 with any of the 19 other natural amino acids (i.e., W, I, M, P, F, G, A, V, L, H, E, K, D, N, Y, C, S, T, or Q) or with non-natural amino acids as are known to those of skill in the art. In some embodiments, said mutation or mutations may comprise substitution of residue R58 with glycine, alanine, valine, cysteine, methionine, serine, threonine, leucine, phenylalanine, tyrosine or histidine. In some embodiments, said mutation or mutations may comprise substitution of residue R58 with alanine, proline or glycine. In some embodiments, said mutation or mutations may comprise any of the substitutions R58G, R58A, R58V. R58C, R58S, or R58F. In some embodiments, said mutation or mutations may comprise the deletion of residue R58.

In some embodiments, said mutation or mutations may comprise substitution of residue P70 of SEQ ID NO: 22 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P70 with lysine, arginine, histidine, threonine, serine, glutamic acid, glutamine, aspartic acid, asparagine, alanine, valine, or glycine. In some embodiments, said mutation or mutations may comprise substitution of residue P70 with alanine, proline or glycine. In some embodiments, said mutation or mutations may comprise any of the substitutions P70K, P70T, P70R, P70E, P70A, or P70G. In some embodiments, said mutation or mutations may comprise the deletion of residue P70.

In some embodiments, said mutation or mutations may comprise substitution of residue P73 of SEQ ID NO: 22 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P70 with lysine, arginine, histidine, threonine, serine, glutamic acid, glutamine, aspartic acid, asparagine, alanine, valine, or glycine. In some embodiments, said mutation or mutations may comprise substitution of residue P73 with alanine, proline or glycine. In some embodiments, said mutation or mutations may comprise any of the substitutions P73R, P73S, P73K, P73N, P73T, P73E, P73D, P73A, or P73G. In some embodiments, said mutation or mutations may comprise the deletion of residue P73.

In some embodiments, said mutation or mutations may comprise substitution of residue G90 of SEQ ID NO: 22 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue G90 with threonine, serine, isoleucine, glutamic acid, glutamine, aspartic acid, asparagine, alanine, valine, or glycine. In some embodiments, said mutation or mutations may comprise substitution of residue G90 with alanine, proline or glycine. In some embodiments, said mutation or mutations may comprise any of the substitutions G90S, G90T, G90I, G90K, G90A, or G90V. In some embodiments, said mutation or mutations may comprise the deletion of residue G90.

In some embodiments, said mutation or mutations may comprise substitution of residue D91 of SEQ ID NO: 22 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue D91 with arginine, lysine, threonine, serine, isoleucine, glutamic acid, glutamine, aspartic acid, asparagine, alanine, methionine, cysteine, valine, or glycine. In some embodiments, said mutation or mutations may comprise substitution of residue D91 with alanine, proline or glycine. In some embodiments, said mutation or mutations may comprise any of the substitutions D91R, D91T, D91I, D91M, D91G, D91A, or D91V. In some embodiments, said mutation or mutations may comprise the deletion of residue D91. In some embodiments, said mutation or mutations may comprise the truncation of SEQ ID NO: 22 N-terminal to residue D91, i.e., said mutation or mutations may comprise an alternative start codon for translation of SEQ ID NO: 22.

In some embodiments, mutations are present at positions 90 and 91. Exemplary combinations of mutations may comprise any of substitutions K90S, K90T, K90I, K90G, K90A, or K90V with any of substitutions D91R, D91T, D91I, D91M, D91G, D91A, or D91V.

In some embodiments, said mutation or mutations may comprise substitution of residue L127 of SEQ ID NO: 22 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue L127 with arginine, lysine, threonine, serine, tyrosine, tryptophan, phenylalanine, histidine, leucine, isoleucine, glutamic acid, glutamine, aspartic acid, asparagine, alanine, methionine, cysteine, valine, proline, or glycine. In some embodiments, said mutation or mutations may comprise substitution of residue L127 with alanine, proline or glycine. In some embodiments, said mutation or mutations may comprise any of the substitutions L127W, L127I, L127M, L127P, L127F, L127G, L127A, L127V, L127H, L127E, L127R, L127K, L127D, L127N, L127Y, L127C, L127S, L127T, or L127Q. In some embodiments, said mutation or mutations may comprise the deletion of residue L127. In some embodiments, said mutation or mutations may comprise the truncation of SEQ ID NO: 22 N-terminal to residue L127, i.e., said mutation or mutations may comprise an alternative start codon for translation of SEQ ID NO: 22. In some embodiments, said mutation or mutations may comprise the truncation of SEQ ID NO: 22 C-terminal to residue L129, i.e., said mutation or mutations may comprise a stop codon terminating translation of SEQ ID NO: 22.

In some embodiments, said mutation or mutations may comprise substitution of residue Q129 of SEQ ID NO: 22 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue Q129 with lysine, arginine, glutamic acid, aspartic acid, asparagine, glutamine, valine, alanine, serine, glycine, or threonine. In some embodiments, said mutation or mutations may comprise substitution of residue Q129 with alanine, proline or glycine. In some embodiments, said mutation or mutations may comprise any of the substitutions Q129K, Q129T, Q129R, Q129E, Q129A, or Q129G. In some embodiments, said mutation or mutations may comprise the deletion of residue Q129.

In some embodiments, said mutation or mutations may comprise substitution of residue Y161 of SEQ ID NO: 22 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue Y161 with lysine, arginine, glutamic acid, aspartic acid, asparagine, glutamine, valine, alanine, serine, proline, glycine, or threonine. In some embodiments, said mutation or mutations may comprise substitution of residue Y161 with alanine, proline or glycine. In some embodiments, said mutation or mutations may comprise any of the substitutions Y161K, Y161T, Y161R, Y161Q, Y161P, Y161E, Y161A, or Y161G. In some embodiments, said mutation or mutations may comprise the deletion of residue Y161.

In some embodiments, said mutation or mutations may comprise substitution of residue K198 of SEQ ID NO: 22 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue K198 with arginine, glutamic acid, aspartic acid, asparagine, glutamine, valine, alanine, serine, proline, glycine, or threonine. In some embodiments, said mutation or mutations may comprise substitution of residue K198 with alanine, proline or glycine. In some embodiments, said mutation or mutations may comprise any of the substitutions K198E, K198A, K198G, K198Q, K198P, or K198R. In some embodiments, said mutation or mutations may comprise the deletion of residue K198.

In some embodiments, said mutation or mutations may comprise substitution of residue M208 of SEQ ID NO: 22 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue M208 with lysine, arginine, glutamic acid, aspartic acid, asparagine, glutamine, valine, alanine, serine, proline, glycine, or threonine. In some embodiments, said mutation or mutations may comprise substitution of residue M208 with alanine, proline or glycine. In some embodiments, said mutation or mutations may comprise any of the substitutions M208S, M208R, M208T, M208P, M208G, or M208A. In some embodiments, said mutation or mutations may comprise the deletion of residue M208 of SEQ ID NO: 22.

In some embodiments, mutations are present at positions 90 and 91. Exemplary combinations of mutations may comprise any of substitutions G90S, G90T, G90I, G90K, G90A, or G90V with any of substitutions D91R, D91T, D91I, D91M, D91G, D91A, or D91V. In some embodiments, said mutations may comprise substitutions at positions G90, D91, and/or L127 of SEQ ID NO: 22 or any combination thereof. In some embodiments, said mutations may optionally comprise any of substitutions G90S, G90T, G90I, G90G, G90A, or G90V, optionally in combination with any of substitutions D91R, D91T, D91I, D91M, D91G, D91A, or D91V and/or any of substitutions L127W, L127I, L127M, L127P, L127F, L127G, L127A, L127V, L127H, L127E, L127R, L127K, L127D, L127N, L127Y, L127C, L127S, L127T, or L127Q. In some embodiments, said mutation or mutations may comprise a combination of mutations at positions 90 and 91 of SEQ ID NO: 22 with the truncation of SEQ ID NO: 22 C-terminal to residue L126, i.e., said mutation or mutations may further comprise a stop codon terminating translation of SEQ ID NO: 22.

In some embodiments, mutations are present at positions 70, 198, and/or 208 of SEQ ID NO: 22. In some embodiments, said mutations may optionally comprise any of substitutions P70K, P70T, P70R, P70E, P70A, or P70G, optionally in combination with any of substitutions K198E, K198A, K198G, K198Q, K198P, or K198R and/or any of substitutions M208S, M208R, M208T, M208P, M208G, or M208A of SEQ ID NO: 22. In some embodiments, said mutations may optionally comprise any of substitutions K198E, K198A, K198G, K198Q, K198P, or K198R, optionally in combination with any of substitutions 73K, P70T, P70R, P70E, P70A, or P70G and/or any of substitutions M208S, M208R, M208T, M208P, M208G, or M208A of SEQ ID NO: 22. In some embodiments, said mutations may optionally comprise any of substitutions M208S, M208R, M208T, M208P, M208G, or M208A, optionally in combination with any of substitutions K198E, K198A, K198G, K198Q, K198P, or K198R and/or any of substitutions P70K, P70T, P70R, P70E, P70A, or P70G of SEQ ID NO: 22.

In some embodiments, mutations are present at positions 58, 161, and/or 208 of SEQ ID NO: 22. In some embodiments, said mutations may optionally comprise any of substitutions R58G, R58A, R58V. R58C, R58S, or R58F, optionally in combination with any of substitutions Y161K, Y161T, Y161R, Y161Q, Y161P, Y161E, Y161A, or Y161G and/or any of substitutions M208S, M208R, M208T, M208P, M208G, or M208A of SEQ ID NO: 22. In some embodiments, said mutations may optionally comprise any of substitutions Y161K, Y161T, Y161R, Y161Q, Y161P, Y161E, Y161A, or Y161G, optionally in combination with any of substitutions R58G, R58A, R58V. R58C, R58S, or R58F, and/or any of substitutions M208S, M208R, M208T, M208P, M208G, or M208A of SEQ ID NO: 22. In some embodiments, said mutations may optionally comprise any of substitutions M208S, M208R, M208T, M208P, M208G, or M208A, optionally in combination with any of substitutions R58G, R58A, R58V. R58C, R58S, or R58F, and/or any of substitutions M208S, M208R, M208T, M208P, M208G, or M208A, of SEQ ID NO: 22.

In some embodiments, mutations are present at positions 58, 73, and/or 129 of SEQ ID NO: 22. In some embodiments, said mutations may optionally comprise any of substitutions R58G, R58A, R58V. R58C, R58S, or R58F, optionally in combination with any of substitutions P73R, P73S, P73K, P73N, P73T, P73E, P73D, P73A, or P73G and/or any of substitutions Q129K, Q129T, Q129R, Q129E, Q129A, or Q129G of SEQ ID NO: 22. In some embodiments, said mutations may optionally comprise any of substitutions P73R, P73S, P73K, P73N, P73T, P73E, P73D, P73A, or P73G, optionally in combination with any of substitutions R58G, R58A, R58V. R58C, R58S, or R58F and/or any of substitutions Q129K, Q129T, Q129R, Q129E, Q129A, or Q129G of SEQ ID NO: 22. In some embodiments, said mutations may optionally comprise any of substitutions Q129K, Q129T, Q129R, Q129E, Q129A, or Q129G, optionally in combination with any of substitutions P73R, P73S, P73K, P73N, P73T, P73E, P73D, P73A, or P73G and/or any of substitutions R58G, R58A, R58V. R58C, R58S, or R58F of SEQ ID NO: 22.

In some embodiments, mutations are present at positions 129, 161, and/or 198 of SEQ ID NO: 22. In some embodiments, said mutations may optionally comprise any of substitutions K198E, K198A, K198G, K198Q, K198P, or K198R, optionally in combination with any of substitutions Y161K, Y161T, Y161R, Y161Q, Y161P, Y161E, Y161A, or Y161G and/or any of substitutions Q129K, Q129T, Q129R, Q129E, Q129A, or Q129G of SEQ ID NO: 22. In some embodiments, said mutations may optionally comprise any of substitutions Y161K, Y161T, Y161R, Y161Q, Y161P, Y161E, Y161A, or Y161G, optionally in combination with any of substitutions K198E, K198A, K198G, K198Q, K198P, or K198R, and/or any of substitutions Q129K, Q129T, Q129R, Q129E, Q129A, or Q129G of SEQ ID NO: 22. In some embodiments, said mutations may optionally comprise any of substitutions Q129K, Q129T, Q129R, Q129E, Q129A, or Q129G, optionally in combination with any of substitutions Y161K, Y161T, Y161R, Y161Q, Y161P, Y161E, Y161A, or Y161G and/or any of substitutions K198E, K198A, K198G, K198Q, K198P, or K198R, of SEQ ID NO: 22.

In some embodiments, the mutation or mutations according to the methods and compositions of the present disclosure may comprise any 1, 2, 3, 4, 5, 6, 7, 8, 9, or 10 of R58G, R58A, R58V. R58C, R58S, R58F, P70K, P70T, P70R, P70E, P70A, P70G, P73R, P73S, P73K, P73N, P73T, P73E, P73D, P73A, P73G, G90S, G90T, G90I, G90G, G90A, G90V, D91R, D91T, D91I, D91M, D91G, D91A, D91V, L127W, L127I, L127M, L127P, L127F, L127G, L127A, L127V, L127H, L127E, L127R, L127K, L127D, L127N, L127Y, L127C, L127S, L127T, L127Q, Q129K, Q129T, Q129R, Q129E, Q129A, Q129G, Y161K, Y161T, Y161R, Y161Q, Y161P, Y161E, Y161A, Y161G, K198E, K198A, K198G, K198Q, K198P, K198R, M208S, M208R, M208T, M208P, M208G, or M208A of SEQ ID NO: 22 or any subset or combination thereof, or any homolog or ortholog thereof.

In some embodiments, said mutation or mutations may comprise one or more substitutions, deletions, or insertions at, or at a position or location surrounding, positions 72, 86, 89, 106, 107, 134, 136, 168, 205, and/or 215 of SEQ ID NO: 23, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue R72 of SEQ ID NO: 23 with any of the 19 other natural amino acids (i.e., W, I, M, P, F, G, A, V, L, H, E, K, D, N, Y, C, S, T, or Q) or with non-natural amino acids as are known to those of skill in the art. In some embodiments, said mutation or mutations may comprise substitution of residue R72 with glycine, alanine, valine, cysteine, methionine, serine, threonine, leucine, phenylalanine, tyrosine or histidine. In some embodiments, said mutation or mutations may comprise substitution of residue R72 with alanine, proline or glycine. In some embodiments, said mutation or mutations may comprise any of the substitutions R72G, R72A, R72V. R72C, R72S, or R72F. In some embodiments, said mutation or mutations may comprise the deletion of residue R72.

In some embodiments, said mutation or mutations may comprise substitution of residue P86 of SEQ ID NO: 23 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P86 with lysine, arginine, histidine, threonine, serine, glutamic acid, glutamine, aspartic acid, asparagine, alanine, valine, or glycine. In some embodiments, said mutation or mutations may comprise substitution of residue P86 with alanine, proline or glycine. In some embodiments, said mutation or mutations may comprise any of the substitutions P86K, P86T, P86R, P86E, P86A, or P86G. In some embodiments, said mutation or mutations may comprise the deletion of residue P86.

In some embodiments, said mutation or mutations may comprise substitution of residue P89 of SEQ ID NO: 23 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P86 with lysine, arginine, histidine, threonine, serine, glutamic acid, glutamine, aspartic acid, asparagine, alanine, valine, or glycine. In some embodiments, said mutation or mutations may comprise substitution of residue P89 with alanine, proline or glycine. In some embodiments, said mutation or mutations may comprise any of the substitutions P89R, P89S, P89K, P89N, P89T, P89E, P89D, P89A, or P89G. In some embodiments, said mutation or mutations may comprise the deletion of residue P89.

In some embodiments, said mutation or mutations may comprise substitution of residue G106 of SEQ ID NO: 23 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue G106 with threonine, serine, isoleucine, glutamic acid, glutamine, aspartic acid, asparagine, alanine, valine, or glycine. In some embodiments, said mutation or mutations may comprise substitution of residue G106 with alanine, proline or glycine. In some embodiments, said mutation or mutations may comprise any of the substitutions G106S, G106T, G106I, G106K, G106A, or G106V. In some embodiments, said mutation or mutations may comprise the deletion of residue G106.

In some embodiments, said mutation or mutations may comprise substitution of residue D107 of SEQ ID NO: 23 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue D107 with arginine, lysine, threonine, serine, isoleucine, glutamic acid, glutamine, aspartic acid, asparagine, alanine, methionine, cysteine, valine, or glycine. In some embodiments, said mutation or mutations may comprise substitution of residue D107 with alanine, proline or glycine. In some embodiments, said mutation or mutations may comprise any of the substitutions D107R, D107T, D107I, D107M, D107G, D107A, or D107V. In some embodiments, said mutation or mutations may comprise the deletion of residue D107. In some embodiments, said mutation or mutations may comprise the truncation of SEQ ID NO: 23 N-terminal to residue D107, i.e., said mutation or mutations may comprise an alternative start codon for translation of SEQ ID NO: 23.

In some embodiments, mutations are present at positions 106 and 107. Exemplary combinations of mutations may comprise any of substitutions K106S, K106T, K106I, K106G, K106A, or K106V with any of substitutions D107R, D107T, D107I, D107M, D107G, D107A, or D107V.

In some embodiments, said mutation or mutations may comprise substitution of residue L134 of SEQ ID NO: 23 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue L134 with arginine, lysine, threonine, serine, tyrosine, tryptophan, phenylalanine, histidine, leucine, isoleucine, glutamic acid, glutamine, aspartic acid, asparagine, alanine, methionine, cysteine, valine, proline, or glycine. In some embodiments, said mutation or mutations may comprise substitution of residue L134 with alanine, proline or glycine. In some embodiments, said mutation or mutations may comprise any of the substitutions L134W, L134I, L134M, L134P, L134F, L134G, L134A, L134V, L134H, L134E, L134R, L134K, L134D, L134N, L134Y, L134C, L134S, L134T, or L134Q. In some embodiments, said mutation or mutations may comprise the deletion of residue L134. In some embodiments, said mutation or mutations may comprise the truncation of SEQ ID NO: 23 N-terminal to residue L134, i.e., said mutation or mutations may comprise an alternative start codon for translation of SEQ ID NO: 23. In some embodiments, said mutation or mutations may comprise the truncation of SEQ ID NO: 23 C-terminal to residue L133, i.e., said mutation or mutations may comprise a stop codon terminating translation of SEQ ID NO: 23.

In some embodiments, said mutation or mutations may comprise substitution of residue Q136 of SEQ ID NO: 23 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue Q136 with lysine, arginine, glutamic acid, aspartic acid, asparagine, glutamine, valine, alanine, serine, glycine, or threonine. In some embodiments, said mutation or mutations may comprise substitution of residue Q136 with alanine, proline or glycine. In some embodiments, said mutation or mutations may comprise any of the substitutions Q136K, Q136T, Q136R, Q136E, Q136A, or Q136G. In some embodiments, said mutation or mutations may comprise the deletion of residue Q136.

In some embodiments, said mutation or mutations may comprise substitution of residue Y168 of SEQ ID NO: 23 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue Y168 with lysine, arginine, glutamic acid, aspartic acid, asparagine, glutamine, valine, alanine, serine, proline, glycine, or threonine. In some embodiments, said mutation or mutations may comprise substitution of residue Y168 with alanine, proline or glycine. In some embodiments, said mutation or mutations may comprise any of the substitutions Y168K, Y168T, Y168R, Y168Q, Y168P, Y168E, Y168A, or Y168G. In some embodiments, said mutation or mutations may comprise the deletion of residue Y168.

In some embodiments, said mutation or mutations may comprise substitution of residue K205 of SEQ ID NO: 23 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue K205 with lysine, arginine, glutamic acid, aspartic acid, asparagine, glutamine, valine, alanine, serine, proline, glycine, or threonine. In some embodiments, said mutation or mutations may comprise substitution of residue K205 with alanine, proline or glycine. In some embodiments, said mutation or mutations may comprise any of the substitutions K205E, K205A, K205G, K205Q, K205P, or K205R. In some embodiments, said mutation or mutations may comprise the deletion of residue K205.

In some embodiments, said mutation or mutations may comprise substitution of residue M215 of SEQ ID NO: 23 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue M215 with lysine, arginine, glutamic acid, aspartic acid, asparagine, glutamine, valine, alanine, serine, proline, glycine, or threonine. In some embodiments, said mutation or mutations may comprise substitution of residue M215 with alanine, proline or glycine. In some embodiments, said mutation or mutations may comprise any of the substitutions M215S, M215R, M215T, M215P, M215G, or M215A. In some embodiments, said mutation or mutations may comprise the deletion of residue M215 of SEQ ID NO: 23.

In some embodiments, mutations are present at positions 106 and 107. Exemplary combinations of mutations may comprise any of substitutions G106S, G106T, G106I, G106K, G106A, or K106V with any of substitutions D107R, D107T, D107I, D107M, D107G, D107A, or D107V. In some embodiments, said mutations may comprise substitutions at positions K106, D107, and/or L134 of SEQ ID NO: 23 or any combination thereof. In some embodiments, said mutations may optionally comprise any of substitutions G106S, G106T, G106I, G106K, G106A, or G106V, optionally in combination with any of substitutions D107R, D107T, D107I, D107M, D107G, D107A, or D107V and/or any of substitutions L134W, L134I, L134M, L134P, L134F, L134G, L134A, L134V, L134H, L134E, L134R, L134K, L134D, L134N, L134Y, L134C, L134S, L134T, or L134Q. In some embodiments, said mutation or mutations may comprise a combination of mutations at positions 106 and 107 of SEQ ID NO: 23 with the truncation of SEQ ID NO: 23 C-terminal to residue L133, i.e., said mutation or mutations may further comprise a stop codon terminating translation of SEQ ID NO: 23.

In some embodiments, mutations are present at positions 86, 205, and/or 215 of SEQ ID NO: 23. In some embodiments, said mutations may optionally comprise any of substitutions 86K, P86T, P86R, P86E, P86A, or P86G, optionally in combination with any of substitutions K205E, K205A, K205G, K205Q, K205P, or K205R and/or any of substitutions M215S, M215R, M215T, M215P, M215G, or M215A of SEQ ID NO: 23. In some embodiments, said mutations may optionally comprise any of substitutions K205E, K205A, K205G, K205Q, K205P, or K205R, optionally in combination with any of substitutions 86K, P86T, P86R, P86E, P86A, or P86G and/or any of substitutions M215S, M215R, M215T, M215P, M215G, or M215A of SEQ ID NO: 23. In some embodiments, said mutations may optionally comprise any of substitutions M215S, M215R, M215T, M215P, M215G, or M215A, optionally in combination with any of substitutions K205E, K205A, K205G, K205Q, K205P, or K205R and/or any of substitutions 86K, P86T, P86R, P86E, P86A, or P86G of SEQ ID NO: 23.

In some embodiments, mutations are present at positions 72, 168, and/or 215 of SEQ ID NO: 23. In some embodiments, said mutations may optionally comprise any of substitutions R72G, R72A, R72V. R72C, R72S, or R72F, optionally in combination with any of substitutions Y168K, Y168T, Y168R, Y168Q, Y168P, Y168E, Y168A, or Y168G and/or any of substitutions M215S, M215R, M215T, M215P, M215G, or M215A of SEQ ID NO: 23. In some embodiments, said mutations may optionally comprise any of substitutions Y168K, Y168T, Y168R, Y168Q, Y168P, Y168E, Y168A, or Y168G, optionally in combination with any of substitutions R72G, R72A, R72V. R72C, R72S, or R72F, and/or any of substitutions M215S, M215R, M215T, M215P, M215G, or M215A of SEQ ID NO: 23. In some embodiments, said mutations may optionally comprise any of substitutions M215S, M215R, M215T, M215P, M215G, or M215A, optionally in combination with any of substitutions R72G, R72A, R72V. R72C, R72S, or R72F, and/or any of substitutions M215S, M215R, M215T, M215P, M215G, or M215A, of SEQ ID NO: 23.

In some embodiments, mutations are present at positions 72, 89, and/or 136 of SEQ ID NO: 23. In some embodiments, said mutations may optionally comprise any of substitutions R72G, R72A, R72V. R72C, R72S, or R72F, optionally in combination with any of substitutions P89R, P89S, P89K, P89N, P89T, P89E, P89D, P89A, or P89G and/or any of substitutions Q136K, Q136T, Q136R, Q136E, Q136A, or Q136G of SEQ ID NO: 23. In some embodiments, said mutations may optionally comprise any of substitutions P89R, P89S, P89K, P89N, P89T, P89E, P89D, P89A, or P89G, optionally in combination with any of substitutions R72G, R72A, R72V. R72C, R72S, or R72F and/or any of substitutions Q136K, Q136T, Q136R, Q136E, Q136A, or Q136G of SEQ ID NO: 23. In some embodiments, said mutations may optionally comprise any of substitutions Q136K, Q136T, Q136R, Q136E, Q136A, or Q136G, optionally in combination with any of substitutions P89R, P89S, P89K, P89N, P89T, P89E, P89D, P89A, or P89G and/or any of substitutions R72G, R72A, R72V. R72C, R72S, or R72F of SEQ ID NO: 23.

In some embodiments, mutations are present at positions 136, 168, and/or 205 of SEQ ID NO: 23. In some embodiments, said mutations may optionally comprise any of substitutions K205E, K205A, K205G, K205Q, K205P, or K205R, optionally in combination with any of substitutions Y168K, Y168T, Y168R, Y168Q, Y168P, Y168E, Y168A, or Y168G and/or any of substitutions Q136K, Q136T, Q136R, Q136E, Q136A, or Q136G of SEQ ID NO: 23. In some embodiments, said mutations may optionally comprise any of substitutions Y168K, Y168T, Y168R, Y168Q, Y168P, Y168E, Y168A, or Y168G, optionally in combination with any of substitutions K205E, K205A, K205G, K205Q, K205P, or K205R, and/or any of substitutions Q136K, Q136T, Q136R, Q136E, Q136A, or Q136G of SEQ ID NO: 23. In some embodiments, said mutations may optionally comprise any of substitutions Q136K, Q136T, Q136R, Q136E, Q136A, or Q136G, optionally in combination with any of substitutions Y168K, Y168T, Y168R, Y168Q, Y168P, Y168E, Y168A, or Y168G and/or any of substitutions K205E, K205A, K205G, K205Q, K205P, or K205R, of SEQ ID NO: 23.

In some embodiments, the mutation or mutations according to the methods and compositions of the present disclosure may comprise any 1, 2, 3, 4, 5, 6, 7, 8, 9, or 10 of R72G, R72A, R72V. R72C, R72S, R72F, P86K, P86T, P86R, P86E, P86A, P86G, P89R, P89S, P89K, P89N, P89T, P89E, P89D, P89A, P89G, G106S, G106T, G106I, G106K, G106A, G106V, D107R, D107T, D107I, D107M, D107G, D107A, D107V, L134W, L134I, L134M, L134P, L134F, L134G, L134A, L134V, L134H, L134E, L134R, L134K, L134D, L134N, L134Y, L134C, L134S, L134T, L134Q, Q136K, Q136T, Q136R, Q136E, Q136A, Q136G, Y168K, Y168T, Y168R, Y168Q, Y168P, Y168E, Y168A, Y168G, K205E, K205A, K205G, K205Q, K205P, K205R, M215S, M215R, M215T, M215P, M215G, or M215A of SEQ ID NO: 23 or any subset or combination thereof, or any homolog or ortholog thereof.

In some embodiments, said mutation or mutations may comprise one or more substitutions, deletions, or insertions at, or at a position or location surrounding, positions 78, 92, 95, 112, 113, 149, 151, 183, 220, and/or 230 of SEQ ID NO: 24, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue R78 of SEQ ID NO: 24 with any of the 19 other natural amino acids (i.e., W, I, M, P, F, G, A, V, L, H, E, K, D, N, Y, C, S, T, or Q) or with non-natural amino acids as are known to those of skill in the art. In some embodiments, said mutation or mutations may comprise substitution of residue R78 with glycine, alanine, valine, cysteine, methionine, serine, threonine, leucine, phenylalanine, tyrosine or histidine. In some embodiments, said mutation or mutations may comprise substitution of residue R78 with alanine, proline or glycine. In some embodiments, said mutation or mutations may comprise any of the substitutions R78G, R78A, R78V. R78C, R78S, or R78F. In some embodiments, said mutation or mutations may comprise the deletion of residue R78.

In some embodiments, said mutation or mutations may comprise substitution of residue L92 of SEQ ID NO: 24 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue L92 with lysine, arginine, histidine, threonine, serine, glutamic acid, glutamine, aspartic acid, asparagine, alanine, valine, or glycine. In some embodiments, said mutation or mutations may comprise substitution of residue L92 with alanine, proline or glycine. In some embodiments, said mutation or mutations may comprise any of the substitutions L92K, L92T, L92R, L92E, L92A, or L92G. In some embodiments, said mutation or mutations may comprise the deletion of residue L92.

In some embodiments, said mutation or mutations may comprise substitution of residue P95 of SEQ ID NO: 24 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue L92 with lysine, arginine, histidine, threonine, serine, glutamic acid, glutamine, aspartic acid, asparagine, alanine, valine, or glycine. In some embodiments, said mutation or mutations may comprise substitution of residue P95 with alanine, proline or glycine. In some embodiments, said mutation or mutations may comprise any of the substitutions P95R, P95S, P95K, P95N, P95T, P95E, P95D, P95A, or P95G. In some embodiments, said mutation or mutations may comprise the deletion of residue P95.

In some embodiments, said mutation or mutations may comprise substitution of residue G112 of SEQ ID NO: 24 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue G112 with threonine, serine, isoleucine, glutamic acid, glutamine, aspartic acid, asparagine, alanine, valine, or glycine. In some embodiments, said mutation or mutations may comprise substitution of residue G112 with alanine, proline or glycine. In some embodiments, said mutation or mutations may comprise any of the substitutions G112S, G112T, G112I, G112K, G112A, or G112V. In some embodiments, said mutation or mutations may comprise the deletion of residue G112.

In some embodiments, said mutation or mutations may comprise substitution of residue D113 of SEQ ID NO: 24 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue D113 with arginine, lysine, threonine, serine, isoleucine, glutamic acid, glutamine, aspartic acid, asparagine, alanine, methionine, cysteine, valine, or glycine. In some embodiments, said mutation or mutations may comprise substitution of residue D113 with alanine, proline or glycine. In some embodiments, said mutation or mutations may comprise any of the substitutions D113R, D113T, D113I, D113M, D113G, D113A, or D113V. In some embodiments, said mutation or mutations may comprise the deletion of residue D113. In some embodiments, said mutation or mutations may comprise the truncation of SEQ ID NO: 24 N-terminal to residue D113, i.e., said mutation or mutations may comprise an alternative start codon for translation of SEQ ID NO: 24.

In some embodiments, mutations are present at positions 112 and 113. Exemplary combinations of mutations may comprise any of substitutions G112S, G112T, G112I, G112K, G112A, or G112V with any of substitutions D113R, D113T, D113I, D113M, D113G, D113A, or D113V.

In some embodiments, said mutation or mutations may comprise substitution of residue L149 of SEQ ID NO: 24 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue L149 with arginine, lysine, threonine, serine, tyrosine, tryptophan, phenylalanine, histidine, leucine, isoleucine, glutamic acid, glutamine, aspartic acid, asparagine, alanine, methionine, cysteine, valine, proline, or glycine. In some embodiments, said mutation or mutations may comprise substitution of residue L149 with alanine, proline or glycine. In some embodiments, said mutation or mutations may comprise any of the substitutions L149W, L149I, L149M, L149P, L149F, L149G, L149A, L149V, L149H, L149E, L149R, L149K, L149D, L149N, L149Y, L149C, L149S, L149T, or L149Q. In some embodiments, said mutation or mutations may comprise the deletion of residue L149. In some embodiments, said mutation or mutations may comprise the truncation of SEQ ID NO: 24 N-terminal to residue L149, i.e., said mutation or mutations may comprise an alternative start codon for translation of SEQ ID NO: 24. In some embodiments, said mutation or mutations may comprise the truncation of SEQ ID NO: 24 C-terminal to residue L151, i.e., said mutation or mutations may comprise a stop codon terminating translation of SEQ ID NO: 24.

In some embodiments, said mutation or mutations may comprise substitution of residue Q151 of SEQ ID NO: 24 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue Q151 with lysine, arginine, glutamic acid, aspartic acid, asparagine, glutamine, valine, alanine, serine, glycine, or threonine. In some embodiments, said mutation or mutations may comprise substitution of residue Q151 with alanine, proline or glycine. In some embodiments, said mutation or mutations may comprise any of the substitutions Q151K, Q151T, Q151R, Q151E, Q151A, or Q151G. In some embodiments, said mutation or mutations may comprise the deletion of residue Q151.

In some embodiments, said mutation or mutations may comprise substitution of residue Y183 of SEQ ID NO: 24 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue Y183 with lysine, arginine, glutamic acid, aspartic acid, asparagine, glutamine, valine, alanine, serine, proline, glycine, or threonine. In some embodiments, said mutation or mutations may comprise substitution of residue Y183 with alanine, proline or glycine. In some embodiments, said mutation or mutations may comprise any of the substitutions Y183K, Y183T, Y183R, Y183Q, Y183P, Y183E, Y183A, or Y183G. In some embodiments, said mutation or mutations may comprise the deletion of residue Y183.

In some embodiments, said mutation or mutations may comprise substitution of residue K220 of SEQ ID NO: 24 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue K220 with lysine, arginine, glutamic acid, aspartic acid, asparagine, glutamine, valine, alanine, serine, proline, glycine, or threonine. In some embodiments, said mutation or mutations may comprise substitution of residue K236 with alanine, proline or glycine. In some embodiments, said mutation or mutations may comprise any of the substitutions K220E, K220A, K220G, K220Q, K220P, or K220R. In some embodiments, said mutation or mutations may comprise the deletion of residue K220.

In some embodiments, said mutation or mutations may comprise substitution of residue M230 of SEQ ID NO: 24 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue M230 with lysine, arginine, glutamic acid, aspartic acid, asparagine, glutamine, valine, alanine, serine, proline, glycine, or threonine. In some embodiments, said mutation or mutations may comprise substitution of residue M230 with alanine, proline or glycine. In some embodiments, said mutation or mutations may comprise any of the substitutions M230S, M230R, M230T, M230P, M230G, or M230A. In some embodiments, said mutation or mutations may comprise the deletion of residue M230 of SEQ ID NO: 24.

In some embodiments, mutations are present at positions 112 and 113. Exemplary combinations of mutations may comprise any of substitutions G112S, G112T, G112I, G112K, G112A, or G112V with any of substitutions D113R, D113T, D113I, D113M, D113G, D113A, or D113V. In some embodiments, said mutations may comprise substitutions at positions G112, D113, and/or L149 of SEQ ID NO: 24 or any combination thereof. In some embodiments, said mutations may optionally comprise any of substitutions G112S, G112T, G112I, G112K, G112A, or G112V, optionally in combination with any of substitutions D113R, D113T, D113I, D113M, D113G, D113A, or D113V and/or any of substitutions L149W, L149I, L149M, L149P, L149F, L149G, L149A, L149V, L149H, L149E, L149R, L149K, L149D, L149N, L149Y, L149C, L149S, L149T, or L149Q. In some embodiments, said mutation or mutations may comprise a combination of mutations at positions 112 and 113 of SEQ ID NO: 24 with the truncation of SEQ ID NO: 24 C-terminal to residue L148, i.e., said mutation or mutations may further comprise a stop codon terminating translation of SEQ ID NO: 24. Further exemplary combinations may comprise any of substitutions G112W, G112I, G112M, G112P, G112F, G112A, G112V, G112L, G112F, G112N, G112Y, G112C, G112S, G112T, G112Q, G112H, G112E, G112R, G112K, or G112D, optionally in combination with any of substitutions D113W, D113I, D113M, D113P, D113F, D113G, D113A, D113V, D113L, D113F, D113N, D113Y, D113C, D113S, D113T, D113Q, D113H, D113E, D113R, or D113K, and/or any of substitutions L149W, L149I, L149M, L149P, L149F, L149G, L149A, L149V, L149F, L149N, L149Y, L149C, L149S, L149T, L149Q, L149H, L149E, L149R, L149K, or L149D.

In some embodiments, mutations are present at positions 92, 220, and/or 230 of SEQ ID NO: 24. In some embodiments, said mutations may optionally comprise any of substitutions L92K, L92T, L92R, L92E, L92A, or L92G, optionally in combination with any of substitutions K236E, K236A, K236G, K236Q, K236P, or K236R and/or any of substitutions M230S, M230R, M230T, M230P, M230G, or M230A of SEQ ID NO: 24. In some embodiments, said mutations may optionally comprise any of substitutions K220E, K220A, K220G, K220Q, K220P, or K220R, optionally in combination with any of substitutions 95K, L92T, L92R, L92E, L92A, or L92G and/or any of substitutions M230S, M230R, M230T, M230P, M230G, or M230A of SEQ ID NO: 24. In some embodiments, said mutations may optionally comprise any of substitutions M230S, M230R, M230T, M230P, M230G, or M230A, optionally in combination with any of substitutions K236E, K236A, K236G, K236Q, K236P, or K236R and/or any of substitutions L92K, L92T, L92R, L92E, L92A, or L92G of SEQ ID NO: 24. Further exemplary combinations may comprise any of substitutions L92W, L92I, L92M, L92F, L92G, L92A, L92V, L92L, L92F, L92N, L92Y, L92C, L92S, L92T, L92Q, L92H, L92E, L92R, L92K, or L92D optionally in combination with any of substitutions K220W, K220I, K220M, K220P, K220F, K220G, K220A, K220V, K220L, K220F, K220N, K220Y, K220C, K220S, K220T, K220Q, K220H, K220E, K220R, or K220D, and/or optionally in combination with any of substitutions M230W, M230I, M230P, M230F, M230G, M230A, M230V, M230L, M230F, M230N, M230Y, M230C, M230S, M230T, M230Q, M230H, M230E, M230R, M230K, or M230D.

In some embodiments, mutations are present at positions 78, 183, and/or 230 of SEQ ID NO: 24. In some embodiments, said mutations may optionally comprise any of substitutions R78G, R78A, R78V. R78C, R78S, or R78F, optionally in combination with any of substitutions Y183K, Y183T, Y183R, Y183Q, Y183P, Y183E, Y183A, or Y183G and/or any of substitutions M230S, M230R, M230T, M230P, M230G, or M230A of SEQ ID NO: 24. In some embodiments, said mutations may optionally comprise any of substitutions Y183K, Y183T, Y183R, Y183Q, Y183P, Y183E, Y183A, or Y183G, optionally in combination with any of substitutions R78G, R78A, R78V. R78C, R78S, or R78F, and/or any of substitutions M230S, M230R, M230T, M230P, M230G, or M230A of SEQ ID NO: 24. In some embodiments, said mutations may optionally comprise any of substitutions M230S, M230R, M230T, M230P, M230G, or M230A, optionally in combination with any of substitutions R78G, R78A, R78V. R78C, R78S, or R78F, and/or any of substitutions M230S, M230R, M230T, M230P, M230G, or M230A, of SEQ ID NO: 24. Further exemplary combinations may comprise any of substitutions R78W, R78I, R78M, R78P, R78F, R78G, R78A, R78V, R78L, R78F, R78N, R78Y, R78C, R78S, R78T, R78Q, R78H, R78E, R78K, or R78D, optionally in combination with any of substitutions M230W, M230I, M230P, M230F, M230G, M230A, M230V, M230L, M230F, M230N, M230Y, M230C, M230S, M230T, M230Q, M230H, M230E, M230R, M230K, or M230D, and/or optionally in combination with any of substitutions Y183W, Y1831, Y183M, Y183P, Y183F, Y183G, Y183A, Y183V, Y183L, Y183F, Y183N, Y183C, Y183S, Y183T, Y183Q, Y183H, Y183E, Y183R, Y183K, or Y183D.

In some embodiments, mutations are present at positions 78, 95, and/or 151 of SEQ ID NO: 24. In some embodiments, said mutations may optionally comprise any of substitutions R78G, R78A, R78V. R78C, R78S, or R78F, optionally in combination with any of substitutions P95R, P95S, P95K, P95N, P95T, P95E, P95D, P95A, or P95G and/or any of substitutions Q151K, Q151T, Q151R, Q151E, Q151A, or Q151G of SEQ ID NO: 24. In some embodiments, said mutations may optionally comprise any of substitutions P95R, P95S, P95K, P95N, P95T, P95E, P95D, P95A, or P95G, optionally in combination with any of substitutions R78G, R78A, R78V. R78C, R78S, or R78F and/or any of substitutions Q151K, Q151T, Q151R, Q151E, Q151A, or Q151G of SEQ ID NO: 24. In some embodiments, said mutations may optionally comprise any of substitutions Q151K, Q151T, Q151R, Q151E, Q151A, or Q151G, optionally in combination with any of substitutions P95R, P95S, P95K, P95N, P95T, P95E, P95D, P95A, or P95G and/or any of substitutions R78G, R78A, R78V. R78C, R78S, or R78F of SEQ ID NO: 24. Further exemplary combinations may comprise any of substitutions R78W, R78I, R78M, R78P, R78F, R78G, R78A, R78V, R78L, R78F, R78N, R78Y, R78C, R78S, R78T, R78Q, R78H, R78E, R78K, or R78D, and/or optionally in combination with any of substitutions P95W, P951, P95M, P95F, P95G, P95A, P95V, P95L, P95F, P95N, P95Y, P95C, P95S, P95T, P95Q, P95H, P95E, P95R, P95K, or P95D, and/or optionally in combination with any of substitutions Q151W, Q151I, Q151M, Q151P, Q151F, Q151G, Q151A, Q151V, Q151L, Q151F, Q151N, Q151Y, Q151C, Q151S, Q151T, Q151H, Q151E, Q151R, Q151K, or Q151D.

In some embodiments, mutations are present at positions 151, 183, and/or 220 of SEQ ID NO: 24. In some embodiments, said mutations may optionally comprise any of substitutions K220E, K220A, K220G, K220Q, K220P, or K220R, optionally in combination with any of substitutions Y183K, Y183T, Y183R, Y183Q, Y183P, Y183E, Y183A, or Y183G and/or any of substitutions Q151K, Q151T, Q151R, Q151E, Q151A, or Q151G of SEQ ID NO: 24. In some embodiments, said mutations may optionally comprise any of substitutions Y183K, Y183T, Y183R, Y183Q, Y183P, Y183E, Y183A, or Y183G, optionally in combination with any of substitutions K236E, K236A, K236G, K236Q, K236P, or K236R, and/or any of substitutions Q151K, Q151T, Q151R, Q151E, Q151A, or Q151G of SEQ ID NO: 24. In some embodiments, said mutations may optionally comprise any of substitutions Q151K, Q151T, Q151R, Q151E, Q151A, or Q151G, optionally in combination with any of substitutions Y183K, Y183T, Y183R, Y183Q, Y183P, Y183E, Y183A, or Y183G and/or any of substitutions K220E, K220A, K220G, K220Q, K220P, or K220R, of SEQ ID NO: 24. Further exemplary combinations may comprise any of substitutions Q151W, Q151I, Q151M, Q151P, Q151F, Q151G, Q151A, Q151V, Q151L, Q151F, Q151N, Q151Y, Q151C, Q151S, Q151T, Q151H, Q151E, Q151R, Q151K, or Q151D, and/or optionally in combination with any of substitutions Y183W, Y1831, Y183M, Y183P, Y183F, Y183G, Y183A, Y183V, Y183L, Y183F, Y183N, Y183C, Y183S, Y183T, Y183Q, Y183H, Y183E, Y183R, Y183K, or Y183D, and/or optionally in combination with any of substitutions K220W, K220I, K220M, K220P, K220F, K220G, K220A, K220V, K220L, K220F, K220N, K220Y, K220C, K220S, K220T, K220Q, K220H, K220E, K220R, or K220D.

In some embodiments, the mutation or mutations according to the methods and compositions of the present disclosure may comprise any 1, 2, 3, 4, 5, 6, 7, 8, 9, or 10 of R78G, R78A, R78V. R78C, R78S, R78F, L92K, L92T, L92R, L92E, L92A, L92G, P95R, P95S, P95K, P95N, P95T, P95E, P95D, P95A, P95G, G112S, G112T, G112I, G112K, G112A, G112V, D113R, D113T, D113I, D113M, D113G, D113A, D113V, L149W, L149I, L149M, L149P, L149F, L149G, L149A, L149V, L149H, L149E, L149R, L149K, L149D, L149N, L149Y, L149C, L149S, L149T, L149Q, Q151K, Q151T, Q151R, Q151E, Q151A, Q151G, Y183K, Y183T, Y183R, Y183Q, Y183P, Y183E, Y183A, Y183G, K220E, K220A, K220G, K220Q, K220P, K220R, M230S, M230R, M230T, M230P, M230G, or M230A of SEQ ID NO: 24 or any subset or combination thereof, or any homolog or ortholog thereof. In some embodiments, the mutation or mutations according to the methods and compositions of the present disclosure may comprise any 1, 2, 3, 4, 5, 6, 7, 8, 9, or 10 of R78W, R78I, R78M, R78P, R78F, R78G, R78A, R78V, R78L, R78F, R78N, R78Y, R78C, R78S, R78T, R78Q, R78H, R78E, R78K, R78D, L92W, L92I, L92M, L92F, L92G, L92A, L92V, L92L, L92F, L92N, L92Y, L92C, L92S, L92T, L92Q, L92H, L92E, L92R, L92K, L92D, P95W, P951, P95M, P95F, P95G, P95A, P95V, P95L, P95F, P95N, P95Y, P95C, P95S, P95T, P95Q, P95H, P95E, P95R, P95K, P95D, G112W, G112I, G112M, G112P, G112F, G112A, G112V, G112L, G112F, G112N, G112Y, G112C, G112S, G112T, G112Q, G112H, G112E, G112R, G112K, G112D, D113W, D113I, D113M, D113P, D113F, D113G, D113A, D113V, D113L, D113F, D113N, D113Y, D113C, D113S, D113T, D113Q, D113H, D113E, D113R, D113K, L149W, L149I, L149M, L149P, L149F, L149G, L149A, L149V, L149F, L149N, L149Y, L149C, L149S, L149T, L149Q, L149H, L149E, L149R, L149K, L149D, Q151W, Q151I, Q151M, Q151P, Q151F, Q151G, Q151A, Q151V, Q151L, Q151F, Q151N, Q151Y, Q151C, Q151S, Q151T, Q151H, Q151E, Q151R, Q151K, Q151D, Y183W, Y1831, Y183M, Y183P, Y183F, Y183G, Y183A, Y183V, Y183L, Y183F, Y183N, Y183C, Y183S, Y183T, Y183Q, Y183H, Y183E, Y183R, Y183K, Y183D, M230W, M230I, M230P, M230F, M230G, M230A, M230V, M230L, M230F, M230N, M230Y, M230C, M230S, M230T, M230Q, M230H, M230E, M230R, M230K, M230D, K220W, K220I, K220M, K220P, K220F, K220G, K220A, K220V, K220L, K220F, K220N, K220Y, K220C, K220S, K220T, K220Q, K220H, K220E, K220R, or K220D of SEQ ID NO: 24 or any subset or combination thereof, or any homolog or ortholog thereof.

In some embodiments, said mutation or mutations may comprise one or more substitutions, deletions, or insertions at, or at a position or location surrounding, positions 80, 94, 97, 114, 115, 151, 153, 185, 222, and/or 232 of SEQ ID NO: 25, or any combination thereof, or homologs or orthologs thereof. In some embodiments, said mutation or mutations may comprise substitution of said residues with any of the 19 other natural amino acids (i.e., W, I, M, P, F, G, A, V, L, H, R, K, D, N, Y, C, S, T, or Q) or with non-natural amino acids as are known to those of skill in the art.

In some embodiments, said mutation or mutations may comprise substitution of residue R80 of SEQ ID NO: 25 with any of the 19 other natural amino acids (i.e., W, I, M, P, F, G, A, V, L, H, E, K, D, N, Y, C, S, T, or Q) or with non-natural amino acids as are known to those of skill in the art. In some embodiments, said mutation or mutations may comprise substitution of residue R80 with glycine, alanine, valine, cysteine, methionine, serine, threonine, leucine, phenylalanine, tyrosine or histidine. In some embodiments, said mutation or mutations may comprise substitution of residue R80 with alanine, proline or glycine. In some embodiments, said mutation or mutations may comprise any of the substitutions R80G, R80A, R80V. R80C, R80S, or R80F. In some embodiments, said mutation or mutations may comprise the deletion of residue R80.

In some embodiments, said mutation or mutations may comprise substitution of residue P94 of SEQ ID NO: 25 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P94 with lysine, arginine, histidine, threonine, serine, glutamic acid, glutamine, aspartic acid, asparagine, alanine, valine, or glycine. In some embodiments, said mutation or mutations may comprise substitution of residue P94 with alanine, proline or glycine. In some embodiments, said mutation or mutations may comprise any of the substitutions P94K, P94T, P94R, P94E, P94A, or P94G. In some embodiments, said mutation or mutations may comprise the deletion of residue P94.

In some embodiments, said mutation or mutations may comprise substitution of residue P97 of SEQ ID NO: 25 with any of the 19 other natural amino acids (i.e., W, I, M, F, G, A, V, L, H, E, R, K, D, N, Y, C, S, T, or Q) or with non-natural amino acids as are known to those of skill in the art. In some embodiments, said mutation or mutations may comprise substitution of residue P94 with lysine, arginine, histidine, threonine, serine, glutamic acid, glutamine, aspartic acid, asparagine, alanine, valine, or glycine. In some embodiments, said mutation or mutations may comprise substitution of residue P97 with alanine, proline or glycine. In some embodiments, said mutation or mutations may comprise any of the substitutions P97R, P97S, P97K, P97N, P97T, P97E, P97D, P97A, or P97G. In some embodiments, said mutation or mutations may comprise the deletion of residue P97.

In some embodiments, said mutation or mutations may comprise substitution of residue G114 of SEQ ID NO: 25 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue G114 with threonine, serine, isoleucine, glutamic acid, glutamine, aspartic acid, asparagine, alanine, valine, or glycine. In some embodiments, said mutation or mutations may comprise substitution of residue G114 with alanine, proline or glycine. In some embodiments, said mutation or mutations may comprise any of the substitutions G114S, G114T, G114I, G114K, G114A, or G114V. In some embodiments, said mutation or mutations may comprise the deletion of residue G114.

In some embodiments, said mutation or mutations may comprise substitution of residue D115 of SEQ ID NO: 25 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue D115 with arginine, lysine, threonine, serine, isoleucine, glutamic acid, glutamine, aspartic acid, asparagine, alanine, methionine, cysteine, valine, or glycine. In some embodiments, said mutation or mutations may comprise substitution of residue D115 with alanine, proline or glycine. In some embodiments, said mutation or mutations may comprise any of the substitutions D115R, D115T, D115I, D115M, D115G, D115A, or D115V. In some embodiments, said mutation or mutations may comprise the deletion of residue D115. In some embodiments, said mutation or mutations may comprise the truncation of SEQ ID NO: 25 N-terminal to residue D115, i.e., said mutation or mutations may comprise an alternative start codon for translation of SEQ ID NO: 25.

In some embodiments, mutations are present at positions 114 and 115. Exemplary combinations of mutations may comprise any of substitutions G114S, G114T, G114I, G114K, G114A, or G114V with any of substitutions D115R, D115T, D115I, D115M, D115G, D115A, or D115V.

In some embodiments, said mutation or mutations may comprise substitution of residue L151 of SEQ ID NO: 25 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue L151 with arginine, lysine, threonine, serine, tyrosine, tryptophan, phenylalanine, histidine, leucine, isoleucine, glutamic acid, glutamine, aspartic acid, asparagine, alanine, methionine, cysteine, valine, proline, or glycine. In some embodiments, said mutation or mutations may comprise substitution of residue L151 with alanine, proline or glycine. In some embodiments, said mutation or mutations may comprise any of the substitutions L151W, L151I, L151M, L151P, L151F, L151G, L151A, L151V, L151H, L151E, L151R, L151K, L151D, L151N, L151Y, L151C, L151S, L151T, or L151Q. In some embodiments, said mutation or mutations may comprise the deletion of residue L151. In some embodiments, said mutation or mutations may comprise the truncation of SEQ ID NO: 25 N-terminal to residue L151, i.e., said mutation or mutations may comprise an alternative start codon for translation of SEQ ID NO: 25. In some embodiments, said mutation or mutations may comprise the truncation of SEQ ID NO: 25 C-terminal to residue L151, i.e., said mutation or mutations may comprise a stop codon terminating translation of SEQ ID NO: 25.

In some embodiments, said mutation or mutations may comprise substitution of residue Q153 of SEQ ID NO: 25 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue Q153 with lysine, arginine, glutamic acid, aspartic acid, asparagine, glutamine, valine, alanine, serine, glycine, or threonine. In some embodiments, said mutation or mutations may comprise substitution of residue Q153 with alanine, proline or glycine. In some embodiments, said mutation or mutations may comprise any of the substitutions Q153K, Q153T, Q153R, Q153E, Q153A, or Q153G. In some embodiments, said mutation or mutations may comprise the deletion of residue Q153.

In some embodiments, said mutation or mutations may comprise substitution of residue Y185 of SEQ ID NO: 25 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue Y185 with lysine, arginine, glutamic acid, aspartic acid, asparagine, glutamine, valine, alanine, serine, proline, glycine, or threonine. In some embodiments, said mutation or mutations may comprise substitution of residue Y185 with alanine, proline or glycine. In some embodiments, said mutation or mutations may comprise any of the substitutions Y185K, Y185T, Y185R, Y185Q, Y185P, Y185E, Y185A, or Y185G. In some embodiments, said mutation or mutations may comprise the deletion of residue Y185.

In some embodiments, said mutation or mutations may comprise substitution of residue K222 of SEQ ID NO: 25 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue K222 with lysine, arginine, glutamic acid, aspartic acid, asparagine, glutamine, valine, alanine, serine, proline, glycine, or threonine. In some embodiments, said mutation or mutations may comprise substitution of residue K222 with alanine, proline or glycine. In some embodiments, said mutation or mutations may comprise any of the substitutions K222E, K222A, K222G, K222Q, K222P, or K222R. In some embodiments, said mutation or mutations may comprise the deletion of residue K222.

In some embodiments, said mutation or mutations may comprise substitution of residue M232 of SEQ ID NO: 25 with any of the 19 other natural amino acids (i.e., W, I, M, F, G, A, V, L, H, E, R, P, D, N, Y, C, S, T, or Q) or with non-natural amino acids as are known to those of skill in the art. In some embodiments, said mutation or mutations may comprise substitution of residue M232 with lysine, arginine, glutamic acid, aspartic acid, asparagine, glutamine, valine, alanine, serine, proline, glycine, or threonine. In some embodiments, said mutation or mutations may comprise substitution of residue M232 with alanine, proline or glycine. In some embodiments, said mutation or mutations may comprise any of the substitutions M232S, M232R, M232T, M232P, M232G, or M232A. In some embodiments, said mutation or mutations may comprise the deletion of residue M232 of SEQ ID NO:

In some embodiments, mutations are present at positions 114 and 115. Exemplary combinations of mutations may comprise any of substitutions G114S, G114T, G114I, G114K, G114A, or G114V with any of substitutions D115R, D115T, D115I, D115M, D115G, D115A, or D115V. In some embodiments, said mutations may comprise substitutions at positions G114, D115, and/or L151 of SEQ ID NO: 25 or any combination thereof. In some embodiments, said mutations may optionally comprise any of substitutions G114S, G114T, G114I, G114K, G114A, or G114V, optionally in combination with any of substitutions D115R, D115T, D115I, D115M, D115G, D115A, or D115V and/or any of substitutions L151W, L151I, L151M, L151P, L151F, L151G, L151A, L151V, L151H, L151E, L151R, L151K, L151D, L151N, L151Y, L151C, L151S, L151T, or L151Q. In some embodiments, said mutation or mutations may comprise a combination of mutations at positions 114 and 115 of SEQ ID NO: 25 with the truncation of SEQ ID NO: 25 C-terminal to residue L150, i.e., said mutation or mutations may further comprise a stop codon terminating translation of SEQ ID NO: 25.

In some embodiments, mutations are present at positions 94, 222, and/or 232 of SEQ ID NO: 25. In some embodiments, said mutations may optionally comprise any of substitutions P94K, P94T, P94R, P94E, P94A, or P94G, optionally in combination with any of substitutions K222E, K222A, K222G, K222Q, K222P, or K222R and/or any of substitutions M232S, M232R, M232T, M232P, M232G, or M232A of SEQ ID NO: 25. In some embodiments, said mutations may optionally comprise any of substitutions K222E, K222A, K222G, K222Q, K222P, or K222R, optionally in combination with any of substitutions P94K, P94T, P94R, P94E, P94A, or P94G and/or any of substitutions M232S, M232R, M232T, M232P, M232G, or M232A of SEQ ID NO: 25. In some embodiments, said mutations may optionally comprise any of substitutions M232S, M232R, M232T, M232P, M232G, or M232A, optionally in combination with any of substitutions K222E, K222A, K222G, K222Q, K222P, or K222R and/or any of substitutions P94K, P94T, P94R, P94E, P94A, or P94G of SEQ ID NO: 25.

In some embodiments, mutations are present at positions 80, 185, and/or 232 of SEQ ID NO: 25. In some embodiments, said mutations may optionally comprise any of substitutions R80G, R80A, R80V. R80C, R80S, or R80F, optionally in combination with any of substitutions Y185K, Y185T, Y185R, Y185Q, Y185P, Y185E, Y185A, or Y185G and/or any of substitutions M232S, M232R, M232T, M232P, M232G, or M232A of SEQ ID NO: 25. In some embodiments, said mutations may optionally comprise any of substitutions Y185K, Y185T, Y185R, Y185Q, Y185P, Y185E, Y185A, or Y185G, optionally in combination with any of substitutions R80G, R80A, R80V. R80C, R80S, or R80F, and/or any of substitutions M232S, M232R, M232T, M232P, M232G, or M232A of SEQ ID NO: 25. In some embodiments, said mutations may optionally comprise any of substitutions M232S, M232R, M232T, M232P, M232G, or M232A, optionally in combination with any of substitutions R80G, R80A, R80V. R80C, R80S, or R80F, and/or any of substitutions M232S, M232R, M232T, M232P, M232G, or M232A, of SEQ ID NO: 25.

In some embodiments, mutations are present at positions 80, 97, and/or 153 of SEQ ID NO: 25. In some embodiments, said mutations may optionally comprise any of substitutions R80G, R80A, R80V. R80C, R80S, or R80F, optionally in combination with any of substitutions P97R, P97S, P97K, P97N, P97T, P97E, P97D, P97A, or P97G and/or any of substitutions Q153K, Q153T, Q153R, Q153E, Q153A, or Q153G of SEQ ID NO: 25. In some embodiments, said mutations may optionally comprise any of substitutions P97R, P97S, P97K, P97N, P97T, P97E, P97D, P97A, or P97G, optionally in combination with any of substitutions R80G, R80A, R80V. R80C, R80S, or R80F and/or any of substitutions Q153K, Q153T, Q153R, Q153E, Q153A, or Q153G of SEQ ID NO: 25. In some embodiments, said mutations may optionally comprise any of substitutions Q153K, Q153T, Q153R, Q153E, Q153A, or Q153G, optionally in combination with any of substitutions P97R, P97S, P97K, P97N, P97T, P97E, P97D, P97A, or P97G and/or any of substitutions R80G, R80A, R80V. R80C, R80S, or R80F of SEQ ID NO: 25.

In some embodiments, mutations are present at positions 153, 185, and/or 222 of SEQ ID NO: 25. In some embodiments, said mutations may optionally comprise any of substitutions K222E, K222A, K222G, K222Q, K222P, or K222R, optionally in combination with any of substitutions Y185K, Y185T, Y185R, Y185Q, Y185P, Y185E, Y185A, or Y185G and/or any of substitutions Q153K, Q153T, Q153R, Q153E, Q153A, or Q153G of SEQ ID NO: 25. In some embodiments, said mutations may optionally comprise any of substitutions Y185K, Y185T, Y185R, Y185Q, Y185P, Y185E, Y185A, or Y185G, optionally in combination with any of substitutions K222E, K222A, K222G, K222Q, K222P, or K222R, and/or any of substitutions Q153K, Q153T, Q153R, Q153E, Q153A, or Q153G of SEQ ID NO: 25. In some embodiments, said mutations may optionally comprise any of substitutions Q153K, Q153T, Q153R, Q153E, Q153A, or Q153G, optionally in combination with any of substitutions Y185K, Y185T, Y185R, Y185Q, Y185P, Y185E, Y185A, or Y185G and/or any of substitutions K222E, K222A, K222G, K222Q, K222P, or K222R, of SEQ ID NO: 25.

In some embodiments, the mutation or mutations according to the methods and compositions of the present disclosure may comprise any 1, 2, 3, 4, 5, 6, 7, 8, 9, or 10 of R80G, R80A, R80V. R80C, R80S, R80F, P94K, P94T, P94R, P94E, P94A, P94G, P97R, P97S, P97K, P97N, P97T, P97E, P97D, P97A, P97G, G114S, G114T, G114I, G114K, G114A, G114V, D115R, D115T, D115I, D115M, D115G, D115A, D115V, L151W, L151I, L151M, L151P, L151F, L151G, L151A, L151V, L151H, L151E, L151R, L151K, L151D, L151N, L151Y, L151C, L151S, L151T, L151Q, Q153K, Q153T, Q153R, Q153E, Q153A, Q153G, Y185K, Y185T, Y185R, Y185Q, Y185P, Y185E, Y185A, Y185G, K222E, K222A, K222G, K222Q, K222P, K222R, M232S, M232R, M232T, M232P, M232G, or M232A of SEQ ID NO: 25 or any subset or combination thereof, or any homolog or ortholog thereof.

One of ordinary skill in the art will readily recognize that the methods and compositions of the present disclosure will further include such improvements, modifications, and reasonable equivalents as may be identified by one of ordinary skill. The methods and compositions of the present disclosure may be further illustrated by reference to the following non-limiting examples.

Further provided herein are polypeptides that comprise nucleic acid extension activity. For example, mutant polypeptides possess increased extension activity when provided modified nucleotides relative to a nearest wild-type polypeptide or to a polypeptide that is wild-type for the mutations listed. Increased extension activity variously refers to an increase in reaction kinetics (increased kcat), increased KD, decreased Km, increased kcat/Km ratio, faster turnover rate, higher turnover number, or other metric that is beneficial to the use of the polypeptide for nucleic acid extension with modified nucleotides. The polypeptides described herein often incorporate at least 30% more modified nucleotides than the nearest wild-type polypeptide in total or in a given duration of time. The polypeptides described herein often incorporate at least 10%, 20%, 30%, 50%, 75%, 100%, 125%, 150%, 200%, 500%, more modified nucleotides than the nearest wild-type polypeptide for a fixed amount of time and modified nucleotide concentration. In some cases, the polypeptides described herein incorporate modified nucleotides at least 1.5, 2, 2.5, 5, 10, 15, 20, 25, or at least 50 times faster than the nearest wild-type polypeptide for a fixed amount of time. Such measurements are often measured under conditions such as a set period of time, such as at least, at most, or exactly 1, 2, 3, 5, 8, 10, 15, 20, or more than 20 minutes. Such measurements are often measured under conditions such as a set nucleotide concentration, such as less than 10 uM, 10 uM, 20 μM, 50 uM, 100 uM, 200 μM, 300 μM, 500 μM, or more than 500 μM, or any concentration within the range identified by the previous list.

Different modified nucleotides are compatible with the compositions and methods described herein. Modified nucleotides in some instances comprise modifications to naturally occurring nucleotides, such as modified bases, sugars, phosphates, or other chemical modification. An exemplary modification is a 3′OH blocking group on the sugar. Such blocking groups allow for iterative coupling of individual nucleotides to a growing chain. Examples of blocking groups comprise azido, aminoxy, disulfide, nitrate, or other group that prevents further extension reactions at the 3′ OH group. Such groups are often reversible, and are removed to allow subsequent extension reactions. Additional nucleotide modifications include modified bases in some cases. Modified bases comprise additional linking groups, or detectable moieties in some instances. Detectable moieties variously comprise fluorescent tags, mass tags, capture moieties (e.g., biotin, maltose, folate, or other capture moiety), or other chemical moiety that can be detected. Such modifications optionally allow the detection and identification of the base in a sequencing workflow.

Sequencing by Synthesis Process with Mutant Polypeptides

Systems, methods and compositions disclosed herein are optionally employed in a number of sequencing approaches, such as single molecule sequencing, long read sequencing, or sequencing by synthesis (SBS) workflows. A typical workflow in some instances comprises at least one step up to all of the steps comprising (1) sample acquisition, (2) library preparation, (3) sequencing, and (4) data analysis. The arrangement and presence or absence of steps or sub-steps in the methods vary in some instances consistent with various embodiments of the disclosure herein.

Samples comprising polynucleotides are acquired from any number of sources, such as human, animal, plant, fungal, virus, bacterial, or other biological source. In some cases, samples are from a purely synthetic origin. Samples are often obtained from tissues, fluids, or any other source comprising polynucleotides to be sequenced. Any source comprising polynucleotides is often a suitable source for polynucleotides to be sequenced.

Library preparation generally comprises one or more of the following steps: (1) sample digestion, (2) polyadenylation, (3) adapter ligation, (4) library amplification, (5) target capture/purification, and (6) amplification. Polynucleotide samples are first digested either enzymatically or mechanically to generate small fragments. These fragments are extended using a kinase, a polymerase, ATP, and/or klenow enzyme to add one or more adenines to the ends of the fragments. The fragments are then ligated to adapter sequences that in some instances comprise universal primer sequences, graft sequences, and/or index sequences using enzymes such as a ligase. The library of adapter tagged polynucleotides is then optionally amplified using universal primers and a polymerase. In some embodiments capture probes are used to selectively bind and purify desired sequences from the library. After capture, washing, and release of the enriched polynucleotides, the enriched library is optionally amplified. The library is then loaded onto a surface, such as a flow cell for sequencing by synthesis.

Single strands of the library polynucleotides (target polynucleotides) are often hybridized to complementary grafting regions covalently attached to the synthesis surface, such as by annealing to complementary grafting sequences present on the adapters. These strands often serve as template polynucleotides for sequencing by synthesis. Solid phase amplification is commonly employed in which the non-surface bound end of the single strands hybridize to neighboring graft sequences, and extension generates reverse complement “copies” of the original single strand at sites in close physical proximity to the original strand (“bridging”). This process is often referred to as cluster or clonal generation, which results in clusters of surface-bound polynucleotide sequences corresponding to the reverse complement of the original strand. Denaturation of bridging strands results in clusters of single stranded polynucleotides (original sequence strands and reverse complement strands). In some instances, this process is repeated to increase cluster density. Generally, each cluster represents a polynucleotide (fragment) that is optionally reassembled into the original sequence after sequencing. In some aspects sequencing primers that bind to the target polynucleotide strands are then added, along with four chain terminating fluorescently labeled dNTPs and a polypeptide with polymerase activity described herein. These matching, complementary labeled dNTPs are added to the growing primer chain, and the surface is imaged to establish the identity of the added nucleotide at each cluster. Cleavage of the fluorescent label and deprotection of the 3′ sugar OH allows a subsequent cycle to commence; the process is optionally repeated until the entire target polynucleotide has been sequenced. Optionally, the complement strand is also used as a template by utilizing a reverse primer to generate a reverse read. The base call data obtained from the forward read and reverse reads is in some aspects a paired-end read. Images collected at each cycle of synthesis are in some aspects analyzed to make base calls, and the sequence of each fragment is identified. In some instances, multiple fragments are sequenced in such a manner and aligned to generate complete sequences of the original target polynucleotides.

In some cases, data analysis comprises evaluation of base calls for polynucleotides that have been sequenced. In some aspects, sequenced polynucleotide (fragments) are reassembled into larger sequences during data analysis. Data analysis optionally comprises any number of steps for processing or interpreting data acquired using the methods and compositions described herein.

The methods and compositions described herein often result in a decreased analysis time. In some cases the analysis time for a single cycle (includes imaging, wherein all base calls generated for a single nucleotide position) is no more than 8 minutes, no more than 7.5, 7, 6.5, 6, 5.5, 5, 4.5, 4, 3.5, 3, 2.5, or no more than 2.5 minutes. In some cases the analysis time for a single cycle is at least 8 minutes, at least 7.5, 7, 6.5, 6, 5.5, 5, 4.5, 4, 3.5, 3, 2.5, or at least 2.5 minutes. In some cases the analysis time for a single cycle is about 8 minutes, about 7.5, 7, 6.5, 6, 5.5, 5, 4.5, 4, 3.5, 3, 2.5, or about 2.5 minutes. In some cases the analysis time for a single cycle is about 2 minutes to about 10 minutes, about 2 minutes to about 8 minutes, about 2 minutes to about 6 minutes, about 3 minutes to about 8 minutes, about 3 minutes to about 6 minutes, about 4 minutes to about 7 minutes, about 3 minutes to about 8 minutes or about 4 minutes to about 10 minutes.

The methods and compositions described herein often allow for longer average read lengths (including forward and reverse reads). In some cases the average read length is at least 100 base pairs, at least 125, 150, 175, 200, 225, 250, 275, 300, 325, 350, 375, 400, 425, 450, 475, 500, 550, 600, 650, or at least 700 base pairs. In some cases the average read length is no more than 100 base pairs, no more than 125, 150, 175, 200, 225, 250, 275, 300, 325, 350, 375, 400, 425, 450, 475, 500, 550, 600, 650, or no more than 700 base pairs. In some cases the average read length is about 100 base pairs, about 125, 150, 175, 200, 225, 250, 275, 300, 325, 350, 375, 400, 425, 450, 475, 500, 550, 600, 650, or about 700 base pairs. In some cases the average read length is about 100 to 700 base pairs, about 100 to 600 base pairs, about 100 to 500 base pairs, about 150 to 400 base pairs, about 200 to 350 base pairs, about 250 to 400 base pairs, about 300 to 500 base pairs, about 400 to 650 base pairs, or about 500 to 700 base pairs.

### Discussion of the Accompanying Figures

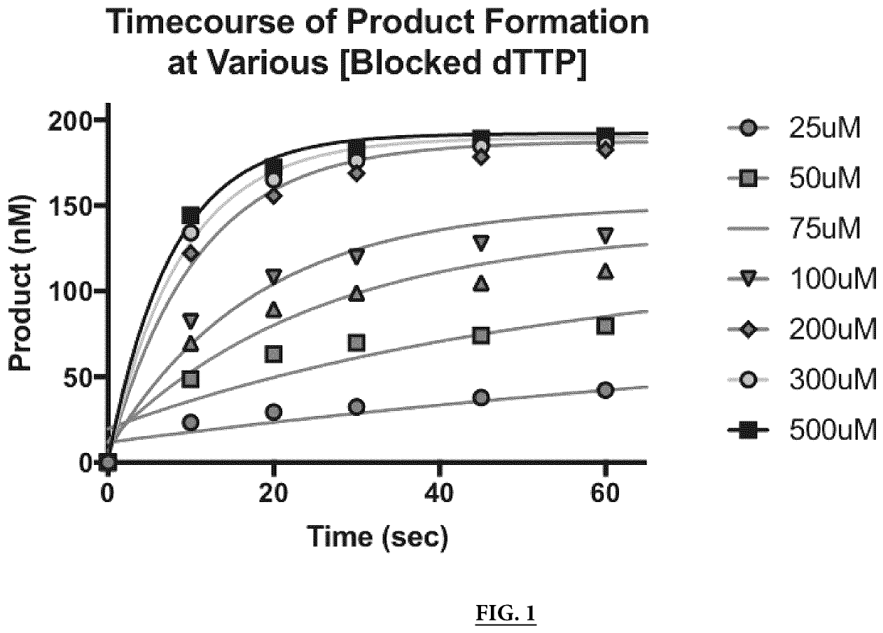

FIG. 1 illustrates the concentration of product (nM) on the Y-axis as a function of time (sec) on the X-axis for the wild-type MMLVRT. The X axis is labeled from 0 to 60 at 20 second intervals, and the Y-axis is labeled from 0 to 200 at 50 nM intervals. The legend on the right side of the graph shows various concentrations of 3′ methylazido dUTP tested: 25 uM, 50 uM, 75 uM, 100 μM, 200 uM, 300 uM, and 500 uM.

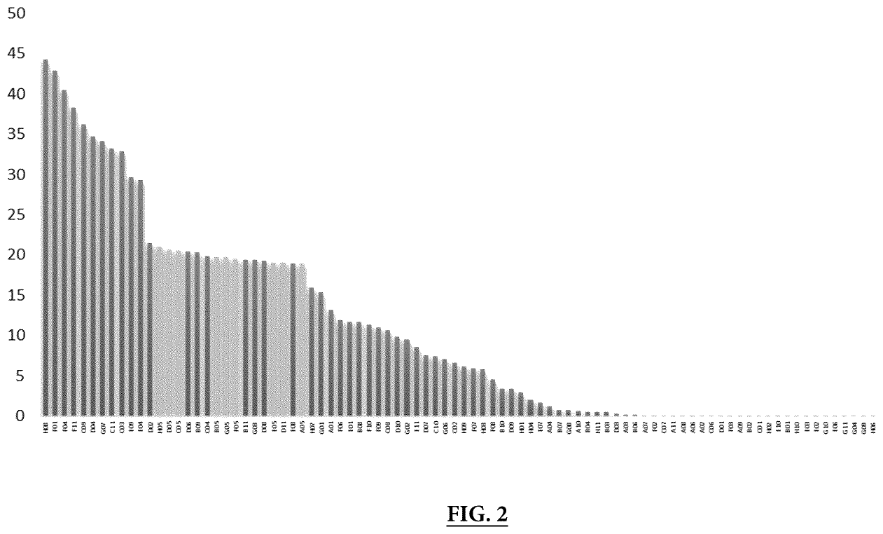

FIG. 2 illustrates the concentration of product (nM) on the Y axis as a function of various transcriptases on the X-axis for the wild-type MMLVRT (light grey bars) and MML VRT mutant (dark grey bars) transcriptases described herein. The X-axis is labeled with 96 well plate locations used to identify the transcriptases, and the Y-axis is labeled from 0 to 50 at 5 nM intervals.

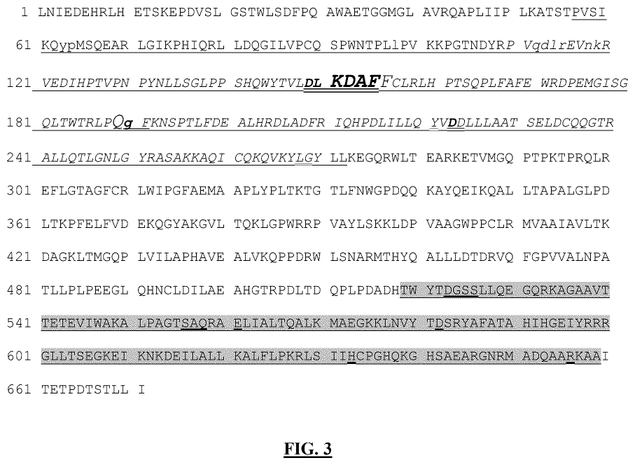

FIG. 3 illustrates an exemplary annotated sequence of MMLV, with a retro-transcriptase-like domain underlined, a DNA binding domain in lower case, a reverse transcriptase domain in italics, the putative active site double underlined, the putative NTP binding site in bold, the RNase exonuclease site highlighted in grey, and the DNA/RNA hybrid binding site in bold underline. Residues replaced by mutations as described herein are marked with large font, and are, in order, K152, D153, A154, F155, F156, and Q190.

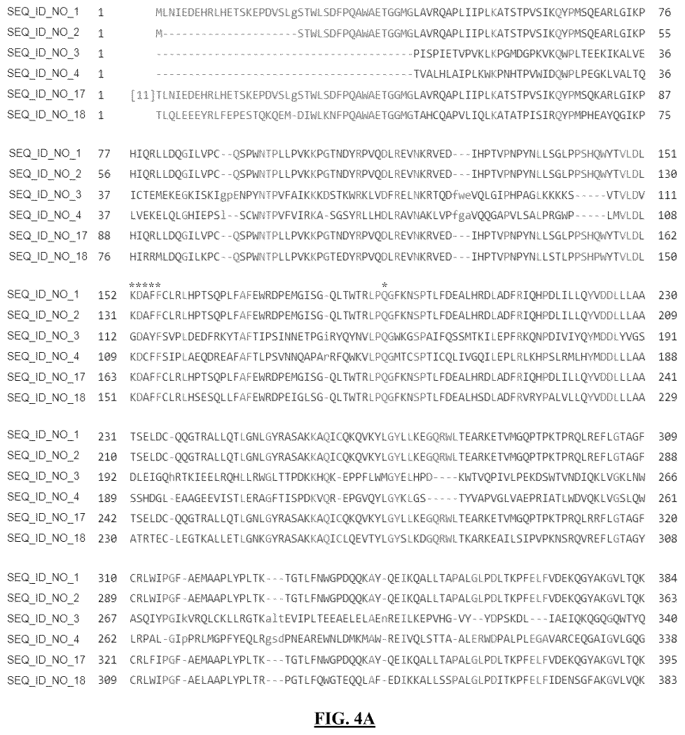

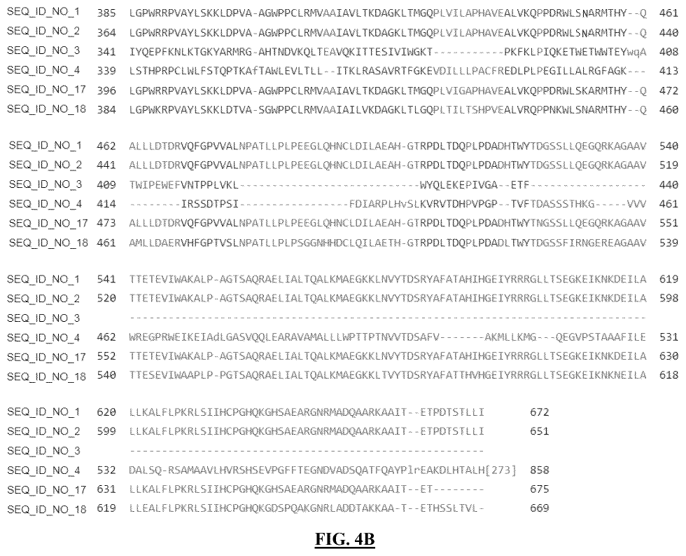

FIG. 4A illustrates an exemplary sequence alignment between SEQ ID NOs: 1-4, 17, and 18 (top to bottom) for residues 1-384 (SEQ ID NO: 1 reference numbering). Residues showing 100% conservation across sequences are shown in red. Residues replaced by mutations as described herein are marked above with an asterisk (*), and are, in order, K152, D153, A154, F155, F156, and Q190. Marking of these residues makes clear that their homologous position in related proteins is readily identified, both for the proteins aligned herein and for other proteins readily identified by one of skill in the art using standard sequence search capabilities such as a BLAST search, available at the NCBI website affiliated with the National Institutes of Health and the National Library of Medicine (ncbi.nlm.nih.gov). Residues showing 100% conservation across sequences are shown in red.

FIG. 4B illustrates an exemplary sequence alignment between SEQ ID NOs: 1-4, 17, and 18 (top to bottom) for residues 385-672 (SEQ ID NO: 1 reference numbering). Residues showing 100% conservation across sequences are shown in red.

### Definitions

As used herein and in the appended claims, the singular forms “a,” “and,” and “the” include plural referents unless the context clearly dictates otherwise. Thus, for example, reference to “polynucleotide” includes a plurality of such polynucleotides and reference to “detecting a nucleotide base” includes reference to one or more methods for detecting nucleotide bases and equivalents thereof known to those skilled in the art, and so forth.

Also, the use of “and” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

The term “sequencing read” as used herein, refers to a polynucleotide fragment in which the sequence has been determined. The identity of individual bases in the fragment are determined by the process of “base calling”.

The term “nucleotide” as used herein, refers to a molecule comprising an aromatic base, a sugar, and a phosphate. Canonical or non-canonical nucleotides are consistent with use of the term. The phosphate in some instances comprises a monophosphate, diphosphate, or triphosphate, or corresponding phosphate analog. Occasionally, “nucleotide” is used informally to refer to a base in a nucleic acid molecule.

The term “amino acid” as used herein, refers to a molecule comprising an amine, a side chain, and a carboxylic acid or analogue thereof. Canonical or non-canonical amino acids are consistent with use of the term. The amino acid in some instances is part of a larger protein, connected through an amide, ester, thioester, alkyl, amide isostere, or other type of chemical linkage.

The term “polypeptide” as used herein, refers to a molecule comprising two or more amino acids that are chemically linked through an amide bond or equivalent.

“About,” as used herein in reference to a number refers to that number +/−10% of that number. As used in reference to a range, ‘about’ refers to a range having a lower limit 10% less than the indicated lower limit of the range and an upper limit that is 10% greater than the indicated upper limit of the range.

The term “percent identity” refers to a comparison between two nucleic acid or amino acid sequences. Such comparisons are measured using any number of alignment methods known in the art, including but not limited to global (e.g., Needleman-Wunsch algorithm) or local alignments (e.g., Smith-Waterman, Sellers, or other algorithm). Percent identity often refers to the percentage of matching positions of two sequences for a contiguous section of positions, wherein the two sequences are aligned in such a way to maximize matching positions and minimize gaps of non-matching positions. In some instances, alignments are conducted wherein there are no gaps between the two sequences. In some instances, the alignment results in less than 5% gaps, less than 3% gaps, or less than 1% gaps. Additional methods of sequence comparison or alignment are also consistent with the disclosure.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

The following illustrative examples are representative of embodiments of compositions and methods described herein and are not meant to be limiting in any way.

### EXAMPLES

### Example 1: Synthesis and Evaluation of MMLV Library with a 3′ Blocked Nucleotide

A vector comprising an expression cassette (promoter, wild-type MMLVRT gene, and terminator) was transformed into E. coli (DE3) cells, plated, and individual colonies picked for expression in liquid cultures as follows. pJ404-MMLV-RT plasmid was codon optimized and synthesized by ATUM and transformed into NEB 5-alpha competent E. coli (C2987H) using manufacturer's protocol. 52 overlapping oligonucleotides were designed based on the optimized DNA sequence to assemble the MMLV-RT gene. Equal volumes of Q5A Hot Start High-Fidelity 2× Master Mix (New England Biolabs) and oligonucleotide mixture containing 50 nM of each oligonucleotide were PCR amplified for 25 cycles. 0.25 μL of the amplified DNA was then reamplified without purification for 32 cycles using only 100 nM terminal primers to generate the oligonucleotide assembled wild type MMLVRT gene. Individual mutagenic oligonucleotides were substituted for the corresponding native oligonucleotide and PCR amplified using the same protocol to generate mutant MMLVRT. Amplified genes were then assembled into pJ404 plasmid using NEBuilder HiFi DNA Assembly Master Mix (New England Biolabs) and transformed into NEB 5-alpha competent E. coli. A 10 μL aliquot of transformants were serially diluted and plated on LB plates supplemented with carbenicillin to check library diversity. Plates were grown at 37° C. for 16 hours, scraped into 10 mL of LB, and miniprepped without amplification to reduce growth bias. Library DNA was stored at −20° C. for transformation into selection cell line.

After transformation, overnight cultures of individual colonies were diluted, grown to O.D. 0.6, induced with IPTG, and grown overnight to express wild-type MMLVRT. Cultures were then centrifuged to remove media, lysed, and the wild-type MMLVRT protein isolated.

Isolated enzyme was placed in buffer and assayed for activity by its ability to incorporate 3′ blocked methylazido dUTP nucleotide into a primer template. Various concentrations of nucleotide and reaction times were evaluated. Quenching buffer was then added after a set period of time to stop the reactions. Extension rates for various concentrations of wild-type MML VRT were measured and are shown in FIG. 1. Reaction rates for the wild-type enzyme are too slow under conditions suitable for nucleotide extension in a nucleic acid sequencing workflow (>300 uM 3′ blocked methylazido dUTP required).

### Example 2: Synthesis and Evaluation of an MMLV Library

Mutant libraries comprising MMLV genes, each comprising point mutations were generated using standard mutagenesis protocols, and these genes were cloned into vector-based expression cassettes. Vectors were transformed into E. coli (DE3) cells, plated, and individual colonies picked for expression in liquid cultures following the general procedure of Example 1. Overnight cultures of individual colonies were diluted, grown to O.D. 0.6, induced with IPTG, and grown overnight to express mutant reverse transcriptases. Cultures were then centrifuged to remove media, lysed, and the reverse transcriptases isolated.

Isolated reverse transcriptases were placed in buffer and assayed for activity by their ability to incorporate 3′ blocked methylazido dUTP nucleotide into a primer template. Methylazido dUTP was added at 10 uM, and reactions were quenched with quenching buffer after 20 minutes. Extension products were purified on a 15% acrylamide gel, and the percent extended product measured, as shown in FIG. 2. Ratios were also obtained as an average of measurements relative to the rate of the wild-type enzyme at different time intervals and nucleotide concentrations. Data for an exemplary mutant F134N (SEQ ID NO: 1 numbering) is shown in Table 3, showing the ratios of extended product for the F134N mutant vs. the wild type F134 polymerase.

TABLE 3

| Time | | | | | | | |
|---|---|---|---|---|---|---|---|
| (sec) | 25 uM | 50 uM | 75 uM | 100 uM | 200 uM | 300 uM | 500 uM |
| 10 | 0.25 | 0.27 | 0.29 | 0.35 | 0.36 | 0.40 | 0.44 |
| 20 | 0.61 | 0.54 | 0.52 | 0.54 | 0.54 | 0.58 | 0.64 |
| 30 | 0.80 | 0.68 | 0.65 | 0.66 | 0.64 | 0.69 | 0.73 |
| 45 | 0.90 | 0.82 | 0.76 | 0.75 | 0.71 | 0.75 | 0.78 |
| 60 | 1.04 | 0.95 | 0.86 | 0.86 | 0.78 | 0.81 | 0.83 |
| 90 | 1.09 | 1.03 | 0.97 | 0.96 | 0.86 | 0.87 | 0.89 |
| 120 | 1.19 | 1.08 | 1.03 | 1.00 | 0.90 | 0.91 | 0.92 |
| 180 | 1.19 | 1.13 | 1.05 | 1.04 | 0.94 | 0.90 | 0.94 |
| 300 | 1.29 | 1.16 | 1.07 | 1.06 | 0.98 | 0.97 | 0.97 |

Mutant polymerases were identified with rates suitable for a nucleic acid sequencing workflow.

### Example 3: Sequencing by Synthesis

A researcher wishes to sequence a polynucleotide sample using standard sequencing by synthesis methodologies, with modification. The polymerase is replaced with a mutant reverse transcriptase comprising at least 85% identity with SEQ ID NO: 2 and the mutation F134N. After sequencing, the researcher is able to ascertain both the identity of all bases in all polynucleotides (fragments) in the sample, as well as correctly reassemble the plurality of polynucleotide fragments in the sample.

### Example 4: Modification of a Homologous RT Domain

A protein having RT activity is identified and aligned to a protein of FIGS. 4A-4B. Residues at positions homologous to positions K152, D153, A154, F155, F156, and Q190 indicated in FIG. 4A are identified, and a mutation is introduced in at least one of these residues. The resultant protein is assessed for its incorporation of 3′ modified nucleotides in an assay such as the assay described in FIG. 2, and is observed to have increased incorporation.

### Example 5

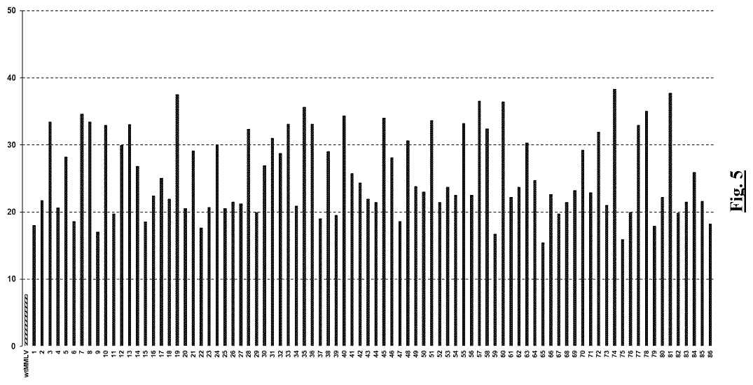

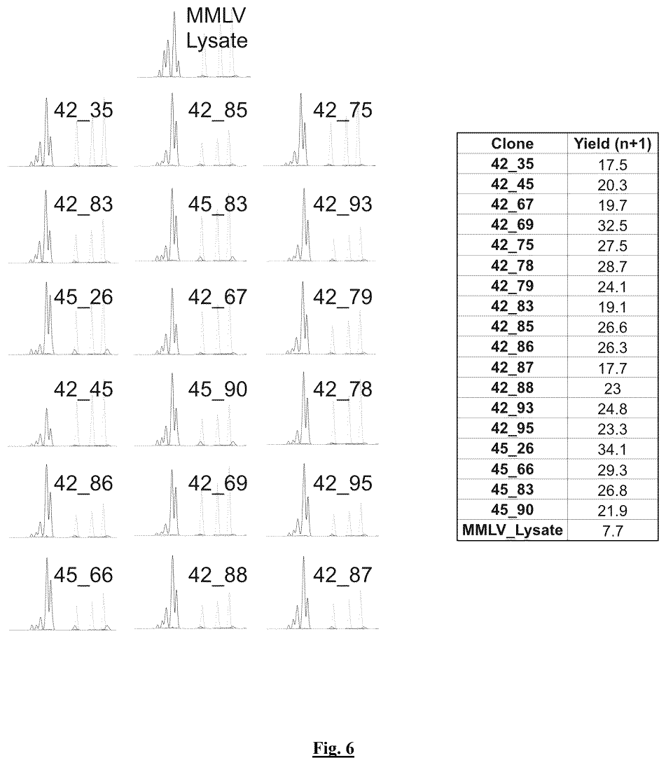

Positions 47, 86, 94, 95, 105, 108, 117, 130, 131, 166, 168, 178, 200, 236, 247, 280, 291, 308, 432, 502, 581, and 585 of SEQ ID NO: 19 were separately mutated to all possible residues by PCR-based site directed mutagenesis using oligonucleotide primers encoding all possible codons at the relevant codon position (FIG. 5 and FIG. 6). The Y-axis represents the percent yield of n+1 extended product for each mutant along the X-axis.

TABLE 4

| Clone | Mutations |
|---|---|
| 42_35 | R94N, Y200P, L247R |
| 42_45 | R94H, Y200G, L247G |
| 42_62 | K236L, L247T |
| 42_67 | K130S, D131H, L166E |
| 42_69 | D131Y, L166T |
| 42_75 | D131R, L166Q |
| 42_78 | K130Q, D131H, L166Y |
| 42_79 | D131H, L166S |
| 42_85 | L166V, Y200L, K236S |
| 42_86 | Q168A, Y200G, K236S |
| 42_93 | L166R, Q168G, Y200A, L247R |
| 45_26 | R94A, P108E, Q168A |
| 45_66 | L166S, D203N, E297K, E350K, A355P |
| 45_83 | L166N, K236R, L247P |
| 45_90 | Q168E, Y200C, K236S |
| 2_15 | R94T, P108T, Q168K |
| 2_21 | R94H, P108T, Q168A |
| 3_7 | R94H, Y200A, L251 |

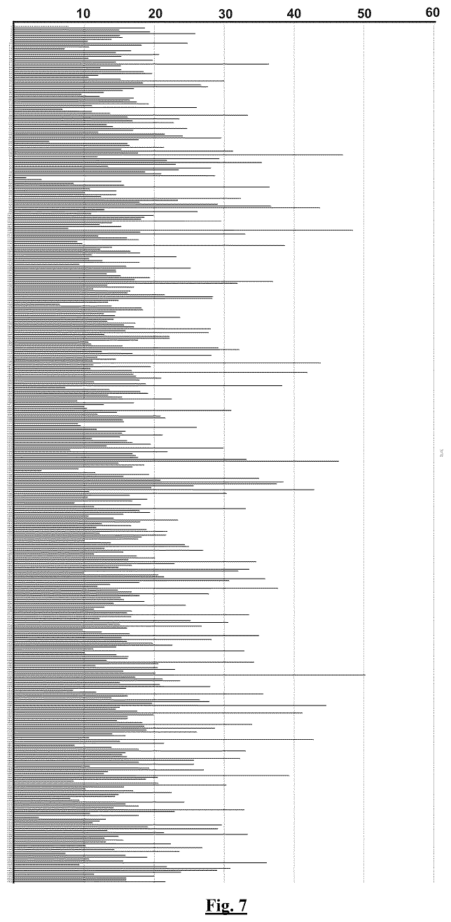

Alternatively, positions 47, 86, 94, 95, 105, 108, 117, 130, 131, 166, 168, 178, 200, 236, 247, 280, 291, 308, 432, 502, 581, and 585 of SEQ ID NO: 19 were mutated in combination by the use of oligonucleotides encoding all possible codons at the relevant codon positions during the assembly of the gene encoding SEQ ID NO. 19. Plasmids encoding mutants were transformed into E. coli cells as described in Example 1, and subjected to screening to identify binding to 3′-blocked nucleotides. Several variants, including mutants shown in Table 4 were shown to have enhanced thermostability and binding of 3′-blocked nucleotides relative to proteins encoded by SEQ ID NO: 19. (FIG. 7). The Y-axis represents the yield of extended n+1 product for each mutant along the X-axis.

### Example 6

Positions 29, 70, 78, 79, 92, 95, 100, 112, 113, 149, 151, 161, 183, 220, 230, 318, 410, and 437 of SEQ ID NO: 24 were separately mutated to all possible residues by PCR-based site directed mutagenesis using oligonucleotide primers encoding all possible codons at the relevant codon position. Alternatively, positions 29, 70, 78, 79, 92, 95, 100, 112, 113, 149, 151, 161, 183, 220, 229, 230 318, 328, 410, and 437 of SEQ ID NO: 24 were mutated in combination by the use of oligonucleotides encoding all possible codons at the relevant codon positions during the assembly of the gene encoding SEQ ID NO. 24. Plasmids encoding mutants were transformed into E. coli cells as described for MMLV-RT in Example 1, and subjected to screening to identify binding to 3′-blocked nucleotides. Several variants, including mutants shown in Table 5 were shown to have enhanced thermostability and binding of 3′-blocked nucleotides relative to proteins encoded by SEQ ID NO: 24.

TABLE 5

| Clone | HIV-RT Mutation |
|---|---|
| 5 | Y318I, A437P |
| 6 | R78A, E79Y, Y183D, K220A, W229F, Y318I, W410E, A437P |
| 11 | L92A, P95R, P170R, R172V, K220A, W229F, Y318W |
| 13 | G112V, D113Q, W229P, Y318K, A437E |
| 15 | R78V, E79W, L92N, P95V |
| 16 | R78E, E79Y, L92P, P95N, L149T, Q151V, Y183S, K220G, |
| | W229Y |
| 17 | L92Y, P95I |
| 20 | R78W, E79W, L92K, P95N, L100V, L149T, Q15IF, W229P |
| 25 | E79S |
| 26 | R78W, E79F, L92N, P95L, Y183W, K220L, W229D, Y328F |
| 28 | Q161P, Y318G |
| 29 | E29R, L149S, Q161L, K220T, W229V, Y319T |
| 30 | Q151L, Y183T, K220R, W229G, Y319W |
| 31 | R78W, E79W, L92K, L100V, L149T, Q151F, Y183D, K220T, |
| | W229P |
| 32 | E29L, R78R, E79P, L92L, P95T, L100N, L149L, Q151D, |
| | Q161A, Y183C, W229R |

### Example 7

### Cell Lysate Preparation:

Colonies were inoculated into 1.5 mL of LB-carbenicillin media with Overnight Express Autoinduction System 1 (Novagen) and grown for 24 hours. Cells were pelleted by centrifuging at 3,500 RCF for 15 min and then resuspended with 200 μL of Bugbuster (MilliporeSigma) with 1 μL of lysonase (MilliporeSigma) diluted to 1/64 of the stock concentration. Cells were allowed to lyse for 15 min at room temperature.

### Blocked-Nucleotide Incorporation Reactions:

Single stranded FAM labeled primer was annealed to unlabeled template containing a 7 nucleotide extension by gradient cooling in annealing buffer (10 mM Tris-HCl PH 7.5, 50 mM NaCl, 1 mM EDTA) from 95° C. to 4° C. The first base to be incorporated on the template determines the blocked nucleotide to be added, i.e. a G on the template would incorporate a C-blocked nucleotide. A reaction master mix comprising 20 nM annealed DNA template and 50 uM d-CTP-azido (Jena Bioscience) or other blocked nucleotide in enzyme dependent buffer was prepared. Individual lysates were diluted 10 fold in the master mix and the reaction was allowed to proceed at 37° C. for 15 min then quenched with 50 mM EDTA in formamide. The quenched reactions were diluted to 500 pM DNA in a standard master mix containing LIZ120 (ThermoFisher Scientific) in formamide for analysis on Applied Biosystems 3500 genetic analyzer. Incorporation of the blocked nucleotide was monitored by using a standard curve generated using single stranded Fam labeled DNA at length 17-27. Purified enzymes were analyzed similarly. Misincorporation was analyzed similarly by using a HEX labeled template with a mismatch first base i.e. a T, C, or A with a blocked-C nucleotide.

Reaction kinetics were monitored by running incorporation reactions and removing 5 μL aliquots into quench buffer in a time series from 0-60 min at varying concentrations of purified enzyme, DNA template, and blocked-nucleotide substrate.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A polypeptide having at least 85% identity to SEQ ID NO: 2, and differing from SEQ ID NO: 2 by at least one of positions K152, D153, A154, F155, F156, and Q190 relative to SEQ ID NO: 1.

2-3. (canceled)

4. The polypeptide of claim 1, wherein the polypeptide comprises a mutation in at least two of positions selected from: K152, D153, A154, F155, F156, and Q190.

5-9. (canceled)

10. The polypeptide of claim 1 wherein the mutation is selected from: K152R, K152C, K152T, and K152L, D153Y, A154Y, F155A, F156N, Q190A, and Q190S.

11-28. (canceled)

29. A polypeptide having at least 85% identity to;
SEQ ID NO: 19, and comprising a mutation in at least one of positions selected from: E47, D86, E95, L117, D178, E280, W291, T308, N432, D502, L581, and E585.

30-31. (canceled)

32. A polypeptide having at least 85% identity to SEQ ID NO: 19 and comprising a mutation selected from R94G, R94A, R94V, R94C, R94S, R94F, P105K, P105T, P105R, P105E, P105A, P105G, P108R, P108S, P108K, P108N, P108T, P108E, P108D, P108A, P108G, K130S, K130T, K130I, K130G, K130A, K130V, D131R, D131T, D131I, D131M, D131G, D131A, D131V, L166W, L166I, L166M, L166P, L166F, L166G, L166A, L166V, L166H, L166E, L166R, L166K, L166D, L166N, L166Y, L166C, L166S, L166T, L166Q, Q168K, Q168T, Q168R, Q168E, Q168A, Q168G, Y200V, Y200C, Y200F, Y200L, Y200S, Y200P, Y200K, Y200T, Y200R, Y200Q, Y200P, Y200E, Y200A, Y200G, K236E, K236A, K236G, K236Q, K236P, K236R, L247S, L247R, L247T, L247P, L247G, L247A, and any combinations thereof.

33-35. (canceled)

36. A polypeptide having at least 85% identity to:
SEQ ID NO: 20 and comprising a mutation selected from: E42K, D77A, Y78A, E86A, L108P, D162N, E264K, E264R, W285F, K291P, T292P, P408K, P409K, R85G, R85A, R85V, R85C, R85S, R85F, P96K, P96T, P96R, P96E, P96A, P96G, P99R, P99S, P99K, P99N, P99T, P99E, P99D, P99A, P99G, K121S, K121T, K121I, K121G, K121A, K121V, D122R, D122T, D122I, D122M, D122G, D122A, D122V, L150W, L150I, L150M, L150P, L150F, L150G, L150A, L150V, L150H, L150E, L150R, L150K, L150D, L150N, L150Y, L150C, L150S, L150T, L150Q, Q152K, Q152T, Q152R, Q152E, Q152A, Q152G, Y184K, Y184T, Y184R, Y184Q, Y184P, Y184E, Y184A, Y184G, K220E, K220A, K220G, K220Q, K220P, K220R, L231S, L231R, L231T, L231P, L231G, L231A, and any combinations thereof;
SEQ ID NO: 21 and comprising a mutation selected from: E47K, D83A, Y84A, E92A, L114P, D175N, E277K, E277R, W288F, K324P, T325P, P421K, A422K, R91, P102, P105, K127, D128, L163, Q165, Y197, K236, K233, L244, R91G, R91A, R91V, R91C, R91S, R91F, P102K, P102T, P102R, P102E, P102A, P102G, P105R, P105S, P105K, P105N, P105T, P105E, P105D, P105A, P105G, K127S, K127T, K127I, K127G, K127A, K127V, D128R, D128T, D128I, D128M, D128G, D128A, D128V, L163W, L163I, L163M, L163P, L163F, L163G, L163A, L163V, L163H, L163E, L163R, L163K, L163D, L163N, L163Y, L163C, L163S, L163T, L163Q, Q165K, Q165T, Q165R, Q165E, Q165A, Q165G, Y197K, Y197T, Y197R, Y197Q, Y197P, Y197E, Y197A, Y197G, K236E, K233E, K233A, K233G, K233Q, K233P, K233R, L244S, L244R, L244T, L244P, L244G, L244A, and any combinations thereof;
SEQ ID NO: 22 and comprising a mutation selected from: E10K, K50A, E59A, L87P, Q139N, Y296K, Y296R, P390F, P391F, A418P, L701K, D702K, N791A, K866W, E868W, T870K, R58G, R58A, R58V, R58C, R58S, R58F, P70K, P70T, P70R, P70E, P70A, P70G, P73R, P73S, P73K, P73N, P73T, P73E, P73D, P73A, P73G, G90S, G90T, G90I, G90G, G90A, G90V, D91R, D91T, D91I, D91M, D91G, D91A, D91V, L127W, L127I, L127M, L127P, L127F, L127G, L127A, L127V, L127H, L127E, L127R, L127K, L127D, L127N, L127Y, L127C, L127S, L127T, L1270, Q129K, Q129T, Q129R, Q129E, Q129A, Q129G, Y161K, Y161T, Y161R, Y161Q, Y161P, Y161E, Y161A, Y161G, K198E, K198A, K198G, K198Q, K198P, K198R, M208S, M208R, M208T, M208P, M208G, M208A, and any combinations thereof;
SEQ ID NO: 23 and comprising a mutation selected from: E27K, D65A, R66A, E73A, L94P, Q146N, Y303K, Y303R, W392F, A419P, V704K, 1705K, K795A, T868W, E872K, R72G, R72A, R72V, R72C, R72S, R72F, P86K, P86T, P86R, P86E, P86A, P86G, P89R, P89S, P89K, P89N, P89T, P89E, P89D, P89A, P89G, G106S, G106T, G106I, G106K, G106A, G106V, D107R, D107T, D107I, D107M, D107G, D107A, D107V, L134W, L134I, L134M, L134P, L134F, L134G, L134A, L134V, L134H, L134E, L134R, L134K, L134D, L134N, L134Y, L134C, L134S, L134T, L134Q, Q136K, Q136T, Q136R, Q136E, Q136A, Q136G, Y168K, Y168T, Y168R, Y168Q, Y168P, Y168E, Y168A, Y168G, K205E, K205A, K205G, K205Q, K205P, K205R, M215S, M215R, M215T, M215P, M215G, M215A, and any combinations thereof;
SEQ ID NO: 24 and comprising a mutation selected from: E29K, K70A, E79A, L100P, Q161N, Y318K, Y318R, W410F, A437P, D443N, E478Q, R78W, R78I, R78M, R78P, R78F, R78G, R78A, R78V, R78L, R78F, R78N, R78Y, R78C, R78S, R78T, R78Q, R78H, R78E, R78K, R78D, L92W, L92I, L92M, L92F, L92G, L92A, L92V, L92L, L92F, L92N, L92Y, L92C, L92S, L92T, L92Q, L92H, L92E, L92R, L92K, L92D, P95W, P951, P95M, P95F, P95G, P95A, P95V, P95L, P95F, P95N, P95Y, P95C, P95S, P95T, P95Q, P95H, P95E, P95R, P95K, P95D, G112W, G112I, G112M, G112P, G112F, G112A, G112V, G112L, G112F, G112N, G112Y, G112C, G112S, G112T, G112Q, G112H, G112E, G112R, G112K, G112D, D113W, D113I, D113M, D113P, D113F, D113G, D113A, D113V, D113L, D113F, D113N, D113Y, D113C, D113S, D113T, D113Q, D113H, D113E, D113R, D113K, L149W, L149I, L149M, L149P, L149F, L149G, L149A, L149V, L149F, L149N, L149Y, L149C, L149S, L149T, L149Q, L149H, L149E, L149R, L149K, L149D, Q151W, Q151I, Q151M, Q151P, Q151F, Q151G, Q151A, Q151V, Q151L, Q151F, Q151N, Q151Y, Q151C, Q151S, Q151T, Q151H, Q151E, Q151R, Q151K, Q151D, Y183W, Y1831, Y183M, Y183P, Y183F, Y183G, Y183A, Y183V, Y183L, Y183F, Y183N, Y183C, Y183S, Y183T, Y183Q, Y183H, Y183E, Y183R, Y183K, Y183D, M230W, M230I, M230P, M230F, M230G, M230A, M230V, M230L, M230F, M230N, M230Y, M230C, M230S, M230T, M230Q, M230H, M230E, M230R, M230K, M230D, K220W, K220I, K220M, K220P, K220F, K220G, K220A, K220V, K220L, K220F, K220N, K220Y, K220C, K220S, K220T, K220Q, K220H, K220E, K220R, K220D, and any combinations thereof; or
SEQ ID NO: 25 and comprising a mutation selected from: E31K, K72A, E81A, L102P, Q163N, Y320K, Y320R, W412F, A439P, P730K, 1731K, T828A, K829A, T832A, V832A, A900W, D904K, R80G, R80A, R80V, R80C, R80S, R80F, P94K, P94T, P94R, P94E, P94A, P94G, P97R, P97S, P97K, P97N, P97T, P97E, P97D, P97A, P97G, G114S, G114T, G114I, G114K, G114A, G114V, D115R, D115T, D115I, D115M, D115G, D115A, D115V, L151W, L151I, L151M, L151P, L151F, L151G, L151A, L151V, L151H, L151E, L151R, L151K, L151D, L151N, L151Y, L151C, L151S, L151T, L151Q, Q153K, Q153T, Q153R, Q153E, Q153A, Q153G, Y185K, Y185T, Y185R, Y185Q, Y185P, Y185E, Y185A, Y185G, K222E, K222A, K222G, K222Q, K222P, K222R, M232S, M232R, M232T, M232P, M232G, M232A, and any combinations thereof.

37-77. (canceled)

78. A mutant nucleic acid polymerase, wherein the nucleic acid polymerase exhibits increased incorporation of 3′ methylazido nucleotide relative to a nearest related wild-type sequence.

79. The nucleic acid polymerase of claim 78, wherein the nucleic acid polymerase sequence differs from a nearest related wild-type sequence by at least one position in an RT domain.

80. The nucleic acid polymerase of claim 78, wherein the nucleic acid polymerase exhibits at least 30% higher incorporation of 3′ methylazido-dUTP relative to a nearest wild type sequence under conditions comprising contacting a polynucleotide template with the nucleic acid polymerase and 10 uM 3′ methylazido-dUTP for 20 minutes.

81-82. (canceled)

83. The nucleic acid polymerase of claim 78, wherein the nucleic acid polymerase exhibits decreased or increased Km of 3′ methylazido-dUTP relative to a nearest related wild-type sequence.

84. (canceled)

85. The nucleic acid polymerase of claim 78, wherein the nucleic acid polymerase is a reverse transcriptase family polymerase, or comprises a reverse transcriptase domain.

86. The nucleic acid polymerase of claim 78, wherein the nucleic acid polymerase comprises a sequence that has at least 85% identity to any one of SEQ ID NOs: 2, and 19-25.

87-94. (canceled)

95. A polypeptide comprising a fragment comprising:
the sequence of SEQ ID NO: 4, wherein the fragment further comprises an adjacent N-terminal aspartic acid, adjacent C-terminal arginine, or a combination thereof;
the sequence of SEQ ID NO: 5, wherein the fragment further comprises an adjacent N-terminal glutamine, adjacent C-terminal histidine, or a combination thereof;
the sequence of SEQ ID NO: 6, wherein the fragment further comprises an adjacent N-terminal valine, adjacent C-terminal cysteine, or a combination thereof;
the sequence of SEQ ID NO: 7, wherein the fragment further comprises an adjacent N-terminal threonine, adjacent C-terminal cysteine, or a combination thereof;
the sequence of SEQ ID NO: 8, wherein the fragment further comprises an adjacent N-terminal threonine, adjacent C-terminal cysteine, or a combination thereof;
the sequence of SEQ ID NO: 9, wherein the fragment further comprises an adjacent N-terminal aspartic acid, adjacent C-terminal leucine, or a combination thereof;
the sequence of SEQ ID NO: 10, wherein the fragment further comprises an adjacent N-terminal aspartic acid, adjacent C-terminal arginine, or a combination thereof;
the sequence of SEQ ID NO: 11, wherein the fragment further comprises an adjacent N-terminal threonine, adjacent C-terminal threonine, or a combination thereof;
the sequence of SEQ ID NO: 12, wherein the fragment further comprises an adjacent N-terminal threonine, adjacent C-terminal asparagine, or a combination thereof.
the sequence of SEQ ID NO: 13, wherein the fragment further comprises an adjacent N-terminal threonine, adjacent C-terminal asparagine, or a combination thereof; or
the sequence of SEQ ID NO: 14, wherein the fragment further comprises an adjacent N-terminal threonine, adjacent C-terminal serine, or a combination thereof.

96-110. (canceled)

111. A method of incorporating a nucleotide analogue into a polynucleotide primer comprising contacting the nucleotide analogue to the polymerase of claim 1.

112. The method of claim 111, wherein the nucleotide analogue comprises a 3′ OH blocking group.

113-116. (canceled)

117. A method of polynucleotide extension, comprising incorporating a nucleotide analogue into a polynucleotide primer using a nucleic acid polymerase, wherein the nucleic acid polymerase exhibits:
an increased incorporation of 3′ methylazido-dUTP relative to a nearest related wild-type sequence;
an increased kcat for incorporation of 3′ methylazido-dfUTP relative to the nearest related wild-type sequence; or
an increased kcat/Km for incorporation of 3′ methylazido-dfUTP relative to the nearest related wild-type sequence.

118. The method of claim 117, wherein the nucleic acid polymerase is a reverse transcriptase.

119. The method of claim 117, wherein the nucleic acid polymerase exhibits at least 10% higher incorporation of 3′ methylazido-dUTP relative to a nearest related wild-type sequence under conditions comprising contacting the nucleic acid polymerase with a polynucleotide primer and 10 uM 3′ methylazido-dUTP for 20 minutes.

120-123. (canceled)

124. (canceled)

125-152. (canceled)

153. A method of sequencing a nucleic acid comprising:
contacting the polypeptide of claim 1 with a nucleotide analogue, a template polynucleotide, and a polynucleotide primer;
incorporating the nucleotide analogue into the polynucleotide primer; and
detecting the incorporation of the nucleotide analogue.

154-166. (canceled)


SEQUENCE LISTING (317 sequences)

SEQ ID NO: 1 - protein, 672 aa, Moloney murine leukemia virus
MLNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 2 - protein, 651 aa, synthetic construct
  REGION 1..651: note = Description of Artificial Sequence: Synthetic polypeptide
MSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 3 - protein, 440 aa, Human immunodeficiency virus
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETF

SEQ ID NO: 4 - protein, 858 aa, Avian myeoblastosis virus
TVALHLAIPLKWKPNHTPVWIDQWPLPEGKLVALTQLVEKELQLGHIEPSLSCWNTPVFVIRKASGSYRLLHDLRAVNAKLVPFGAVQQGAPVLSALPRGWPLMVLDLKDCFFSIPLAEQDREAFAFTLPSVNNQAPARRFQWKVLPQGMTCSPTICQLIVGQILEPLRLKHPSLRMLHYMDDLLLAASSHDGLEAAGEEVISTLERAGFTISPDKVQREPGVQYLGYKLGSTYVAPVGLVAEPRIATLWDVQKLVGSLQWLRPALGIPPRLMGPFYEQLRGSDPNEAREWNLDMKMAWREIVQLSTTAALERWDPALPLEGAVARCEQGAIGVLGQGLSTHPRPCLWLFSTQPTKAFTAWLEVLTLLITKLRASAVRTFGKEVDILLLPACFREDLPLPEGILLALRGFAGKIRSSDTPSIFDIARPLHVSLKVRVTDHPVPGPTVFTDASSSTHKGVVVWREGPRWEIKEIADLGASVQQLEARAVAMALLLWPTTPTNVVTDSAFVAKMLLKMGQEGVPSTAAAFILEDALSQRSAMAAVLHVRSHSEVPGFFTEGNDVADSQATFQAYPLREAKDLHTALHIGPRALSKACNISMQQAREVVQTCPHCNSAPALEAGVNPRGLGPLQIWQTDFTLEPRMAPRSWLAVTVDTASSAIVVTQHGRVTSVAAQHHWATAIAVLGRPKAIKTDNGSCFTSKSTREWLARWGIAHTTGIPGNSQGQAMVERANRLLKDKIRVLAEGDGFMKRIPTSKQGELLAKAMYALNHFERGENTKTPIQKHWRPTVLTEGPPVKIRIETGEWEKGWNVLVWGRGYAAVKNRDTDKVIWVPSRKVKPDIAQKDEVTKKDEASPLFA

SEQ ID NO: 5 - protein, 16 aa, synthetic construct
  REGION 1..16: note = Description of Artificial Sequence: Synthetic peptide
WYTVLDLRDAFFCLRL

SEQ ID NO: 6 - protein, 8 aa, synthetic construct
  REGION 1..8: note = Description of Artificial Sequence: Synthetic peptide
LDLCDAFF

SEQ ID NO: 7 - protein, 9 aa, synthetic construct
  REGION 1..9: note = Description of Artificial Sequence: Synthetic peptide
VLDLSDAFF

SEQ ID NO: 8 - protein, 9 aa, synthetic construct
  REGION 1..9: note = Description of Artificial Sequence: Synthetic peptide
VLDLLDAFF

SEQ ID NO: 9 - protein, 14 aa, synthetic construct
  REGION 1..14: note = Description of Artificial Sequence: Synthetic peptide
TVLDLKYAFFCLRL

SEQ ID NO: 10 - protein, 7 aa, synthetic construct
  REGION 1..7: note = Description of Artificial Sequence: Synthetic peptide
LKDYFFC

SEQ ID NO: 11 - protein, 8 aa, synthetic construct
  REGION 1..8: note = Description of Artificial Sequence: Synthetic peptide
LKDAAFCL

SEQ ID NO: 12 - protein, 15 aa, synthetic construct
  REGION 1..15: note = Description of Artificial Sequence: Synthetic peptide
VLDLKDAFNCLRLHP

SEQ ID NO: 13 - protein, 7 aa, synthetic construct
  REGION 1..7: note = Description of Artificial Sequence: Synthetic peptide
RLPAGFK

SEQ ID NO: 14 - protein, 8 aa, synthetic construct
  REGION 1..8: note = Description of Artificial Sequence: Synthetic peptide
RLPSGFKN

SEQ ID NO: 15 - protein, 216 aa, synthetic construct
  REGION 1..216: note = Description of Artificial Sequence: Synthetic polypeptide
PVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLL

SEQ ID NO: 16 - protein, 141 aa, Moloney murine leukemia virus
TWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAA

SEQ ID NO: 17 - protein, 675 aa, Tumebacillus flagellatus
SSGLVPRGSHMTLNIEDEHRLHETSKEPDVSLGSTWLSDFPQAWAETGGMGLAVRQAPLIIPLKATSTPVSIKQYPMSQKARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLRRFLGTAGFCRLFIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIGAPHAVEALVKQPPDRWLSKARMTHYQALLLDTDRVQFGPVVALNPATLLPLPEEGLQHNCLDILAEAHGTRPDLTDQPLPDADHTWYTNGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITET

SEQ ID NO: 18 - protein, 669 aa, Feline leukemia virus
TLQLEEEYRLFEPESTQKQEMDIWLKNFPQAWAETGGMGTAHCQAPVLIQLKATATPISIRQYPMPHEAYQGIKPHIRRMLDQGILKPCQSPWNTPLLPVKKPGTEDYRPVQDLREVNKRVEDIHPTVPNPYNLLSTLPPSHPWYTVLDLKDAFFCLRLHSESQLLFAFEWRDPEIGLSGQLTWTRLPQGFKNSPTLFDEALHSDLADFRVRYPALVLLQYVDDLLLAAATRTECLEGTKALLETLGNKGYRASAKKAQICLQEVTYLGYSLKDGQRWLTKARKEAILSIPVPKNSRQVREFLGTAGYCRLWIPGFAELAAPLYPLTRPGTLFQWGTEQQLAFEDIKKALLSSPALGLPDITKPFELFIDENSGFAKGVLVQKLGPWKRPVAYLSKKLDTVASGWPPCLRMVAAIAILVKDAGKLTLGQPLTILTSHPVEALVRQPPNKWLSNARMTHYQAMLLDAERVHFGPTVSLNPATLLPLPSGGNHHDCLQILAETHGTRPDLTDQPLPDADLTWYTDGSSFIRNGEREAGAAVTTESEVIWAAPLPPGTSAQRAELIALTQALKMAEGKKLTVYTDSRYAFATTHVHGEIYRRRGLLTSEGKEIKNKNEILALLEALFLPKRLSIIHCPGHQKGDSPQAKGNRLADDTAKKAATETHSSLTVL

SEQ ID NO: 19 - protein, 650 aa, Moloney murine leukemia virus
MTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAALIALTQALKMAEGKKLNVYTASRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 20 - protein, 441 aa, Xenotropic murine leukemia virus-related virus
DFPQAWAETGGMGLAVRQAPLIIPLKATSTPVSIKQYPMSQEARLGIKPHIQRLLDQGILVPCQSPWNTPLLPVKKDYRPVQDLREVNKRVEDIHPTVPNPYNLLSGLPPSHQWYTVLDLKDAFFCLRLHPTSQPLFAFEWRDQLTWTRLPQGFKNSPTLFDEALHRDLADFRIQHPDLILLQYVDDLLLAATSEQDCQRGTRALLQTLGNLGYRASAKKAQICQKQVKYLGYLLKEGQRWLTEARKETVMGQPTPKTPRQLREFLGTAGFCRLWIPGFAEMAAPLYPLTKTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPARMTHYQAMLLDTDRVQFGPVVALNPATLLPLP

SEQ ID NO: 21 - protein, 453 aa, unidentified
  REGION 1..453: note = Description of Unknown: 4mh8 sequence
TWLSDFPQAWAETGGMGLAVRQAPLIIPLKATSTPVSIKQYPMSQEARLGIKPHIQRLLDQGILVPCQSPWNTPLLPVKTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVIKAPHAVEALVKQPPARMTHYQALLLDTDRVQFGPVVALNPATLLPLP

SEQ ID NO: 22 - protein, 935 aa, Feline immunodeficiency virus
QIKQWPLTNEKIEALTEIVERLEREGKVKRADPNNPWNTPVFAIKKKSGKWRMLIDFRELNKLTEKGAQLGLPHPAGLQIKKQVTVLDIGDAYFTIPLDPDYAPYTAFTLPRKNNAGPGRRFVWCSLPQGWILSPLIYQSTLDNIIQPFIRQNPQLDIYQYMDDIYIGSNLSKKEHKEKVEELRKLLLWWGFETPEDKLQEEPPYTWMGYELHPLTWTIQQKQLDIPEQPTLNELQKLAGKINWASQAIPDLSIKALTNMMRGNQNLNSTRQWTKEARLEVQKAKKAIEEQVQLGYYDPSKELYAKLSLVGPHQISYQVYQKDPEKILWYGKMSRQKKKAENTCDIALRACYKIREESIIRIGKEPRYEIPTSREAWESNLINSPYLKAPPPEVEYIHAALNIKRALSMIKDAPIPGAETWYIDGGRKLGKAAKAAYWTDTGKWQVMELEGSNQKAEIQALLLALKAGSEEMNIITDSQYVINIILQQPDMMEGIWQEVLEELEKKTAIFIDWVPGHKGIPGNEEVDKLCSDKIPVVKVKMKDPNKGPQIKQWPLTNEKIEALTEIVERLEREGKVKRADPNNPWNTPVFAIKKKSGKWRMLIDFRELNKLTEKLGLPHPAGLQIKKQVTVLDIGDAYFTIPLDPDYAPYTAFTLPRKNNAGPGRRFVWCSLPQGWILSPLIYQSTLDNIIQPFIRQNPQLDIYQYMDDIYIGSNLSKKEHKEKVEELRKLLLWPEDKLQEETWTIQQKQLDIPEQPTLNELQKLAGKINWASQAIPDLSIKALTNMMRGNQNLNSTRQWTKEARLEVQKAKKAIEEQVQLGYYDPSKELYAKLSLVGPHQISYQVYQKDPEKILWYGKMSRQKKKAENTCDIALRACYKIREESIIRIGKEPRYEIPTSREAWESNLINSPYLKAPPPEVEYIHAALNIKRALS

SEQ ID NO: 23 - protein, 940 aa, Human immunodeficiency virus 2
AKVEPIKIMLKPGKDGPKLRQWPLTKEKIEALKEICEKMEKEGQLEEAPPTNPYNTPTFAIKKKDRMLIDFRELNKVTQDFTEIQLGIPHPAGLAKKRRITVLDVGDAYFSIPLHEDFRPYTAFTLKRYIYKVLPQGWKGSPAIFQHTMRQVLEPFRKANKDVIIIQYMDDILIASDRTDLEHDRVVLQLKELLNGLGFSTPDEKFQKDPPYHWMGYELWPTKWKLQKIQLPQKEIWTVNDIQKLVGVLNWAAQLYPGIKTKHLCRLISGKMTLTEEVQWTELAEAELEENRIILSQEQEGHYYQEEKELEATVQKDQDNQWTYKIHQEEKILKVGKYAKVTHTNGIRLLAQVVQKIGKEALVIWGRIPKFHLPVEREIWEQWWDNYWQVTWIPDWDFVSTPPLVRLAFNLVGDPIPGAETFYTDGSCNRQSKEGKAGYVTDRGKDKVKKLEQTTNQQAELEAFAMALTDSGPKVNIIVDSQYVMGIVASQPTESESKIVNQIIEEMIKKEAIYVAWVPAHKGIGGNQEVDHLVSQGIEPIKIMLKPGKDGPKLRQWPLTKEKIEALKEICEKMEKEGQLEEAPPTNPYNTPTFAIKNKWRMLIDFRELNKVTQDFTEIQPHPAGLAKKRRITVLDVGDAYFSIPLHEDFRPYTAFTLPSVNNAEPGKRYIYKVLPQGWKGSPAIFQHTMRQVLEPFRKANKDVIIIQYMDDILIASDRTDLEHDRVVLQLKELLNGWMGYELWPTKWKLQKIQLPQKEIWTVNDIQKLVGVLNWAAQLYPGIKTKHLCRLISGKMTLTEEVQWTELAEAELEENRIILSQEQEGHYYQEEKELEATVQKDQDNQWTYKIHQEEKILKVGKYAKVKNTHTNGIRLLAQVVQKIGKEALVIWGRIPKFHLPVEREIWEQWWDNYWQVTWIPDWDFVSTPPLVRLAFNLVGD

SEQ ID NO: 24 - protein, 560 aa, unidentified
  REGION 1..560: note = Description of Unknown: Seq3SS sequence
PISPIAPVPVKLKPGMDGPKVKQWPLSKEKIEALTAICQEMEQEGKISRIGPENPYNTPIFAIKKKDSTKWRKLVDFRELNKRTQDFWEVQLGIPHPGGLKQKRSVTVLDVGDAYFSCPLDPDFRKYTAFTIPSVNNETPGIRYQYNVLPQGWKGSPAIFQSSMTKILDPFRKDNPELEICQYMDDLYVGSDLPLAEHRKRVELLREHLYQWGFTTPDKKHQKEPPFMWMGYELHPDKWTVQPIKLPNKDVWTVNDIQKLIGKLNWASQIYQGIRVRELCKLIRGTKSLTEVVPLSKEAEMELEENREKLKEPMHGVYYQPDKDLWVNIQKQGEGQWTYQIYQDEHKNLKTGKYTRQRGAHTNDIRQLAEVIQKVSQESIVIWGKLPKFKLPVTRETWETWWADYWQATWIPEWDYVSTPPLIKLWYRLESEPIMGAETYYVDGAANRDTKLGKAGYVTEQGKQKIIKLNETTNQKAELMAVLLALQDSKEKVNIVTDSQYVLGIISSQPTQSESPIVQQIIEELTKKEQVYLTWVPAHKGIGGNEKIDKLVSKDIRRVL

SEQ ID NO: 25 - protein, 968 aa, Human immunodeficiency virus 1
MVPISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFKKQNPDIVIYQYMDDLYVGSDLEIGQHRTKIEELRQHLLRWGLTTPDKKHQKEPPFLWMGYELHPDKWTVQPIVLPEKDSWTVNDICKLVGKLNWASQIYPGIKVRQLSKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNKGRQKVVPLTNTTNQKTELQAIYLALQDSGLEVNIVTNSQYALGIIQAQPDKSESELVNQIIEQLIKKEKVYLAWVPAHKGIGGNEQVDKLVSAPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFKKQNPDIVIYQYMDDLYVGSDLEIGQHRTKIEELRQHLLRWGLTTPGYELHPDKWTVQPIVLPEKDSWTVNDIQKLVGKLNWASQIYPGIKVRQLSKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQ

SEQ ID NO: 26 - protein, 5 aa, Moloney murine leukemia virus
KDAFF

SEQ ID NO: 27 - protein, 5 aa, Moloney murine leukemia virus
KDAFF

SEQ ID NO: 28 - protein, 5 aa, synthetic construct
  REGION 1..5: note = Description of Artificial Sequence: Synthetic peptide
KDAFF

SEQ ID NO: 29 - protein, 5 aa, synthetic construct
  REGION 1..5: note = Description of Artificial Sequence: Synthetic peptide
KDAFF

SEQ ID NO: 30 - protein, 5 aa, synthetic construct
  REGION 1..5: note = Description of Artificial Sequence: Synthetic peptide
KDAFN

SEQ ID NO: 31 - protein, 5 aa, synthetic construct
  REGION 1..5: note = Description of Artificial Sequence: Synthetic peptide
KDAFN

SEQ ID NO: 32 - protein, 5 aa, synthetic construct
  REGION 1..5: note = Description of Artificial Sequence: Synthetic peptide
KDAFN

SEQ ID NO: 33 - protein, 5 aa, synthetic construct
  REGION 1..5: note = Description of Artificial Sequence: Synthetic peptide
KDAAF

SEQ ID NO: 34 - protein, 5 aa, synthetic construct
  REGION 1..5: note = Description of Artificial Sequence: Synthetic peptide
KDAAF

SEQ ID NO: 35 - protein, 5 aa, synthetic construct
  REGION 1..5: note = Description of Artificial Sequence: Synthetic peptide
KDAAF

SEQ ID NO: 36 - protein, 5 aa, synthetic construct
  REGION 1..5: note = Description of Artificial Sequence: Synthetic peptide
KDAAN

SEQ ID NO: 37 - protein, 5 aa, synthetic construct
  REGION 1..5: note = Description of Artificial Sequence: Synthetic peptide
KDAAN

SEQ ID NO: 38 - protein, 5 aa, synthetic construct
  REGION 1..5: note = Description of Artificial Sequence: Synthetic peptide
KDAAN

SEQ ID NO: 39 - protein, 5 aa, synthetic construct
  REGION 1..5: note = Description of Artificial Sequence: Synthetic peptide
KDYFF

SEQ ID NO: 40 - protein, 5 aa, synthetic construct
  REGION 1..5: note = Description of Artificial Sequence: Synthetic peptide
KDYFF

SEQ ID NO: 41 - protein, 5 aa, synthetic construct
  REGION 1..5: note = Description of Artificial Sequence: Synthetic peptide
KDYFF

SEQ ID NO: 42 - protein, 5 aa, synthetic construct
  REGION 1..5: note = Description of Artificial Sequence: Synthetic peptide
KDYFN

SEQ ID NO: 43 - protein, 5 aa, synthetic construct
  REGION 1..5: note = Description of Artificial Sequence: Synthetic peptide
KDYFN

SEQ ID NO: 44 - protein, 5 aa, synthetic construct
  REGION 1..5: note = Description of Artificial Sequence: Synthetic peptide
KDYFN

SEQ ID NO: 45 - protein, 5 aa, synthetic construct
  REGION 1..5: note = Description of Artificial Sequence: Synthetic peptide
KDYAF

SEQ ID NO: 46 - protein, 5 aa, synthetic construct
  REGION 1..5: note = Description of Artificial Sequence: Synthetic peptide
KDYAF

SEQ ID NO: 47 - protein, 5 aa, synthetic construct
  REGION 1..5: note = Description of Artificial Sequence: Synthetic peptide
KDYAF

SEQ ID NO: 48 - protein, 5 aa, synthetic construct
  REGION 1..5: note = Description of Artificial Sequence: Synthetic peptide
KDYAN

SEQ ID NO: 49 - protein, 5 aa, synthetic construct
  REGION 1..5: note = Description of Artificial Sequence: Synthetic peptide
KDYAN

SEQ ID NO: 50 - protein, 5 aa, synthetic construct
  REGION 1..5: note = Description of Artificial Sequence: Synthetic peptide
KDYAN

SEQ ID NO: 51 - protein, 5 aa, synthetic construct
  REGION 1..5: note = Description of Artificial Sequence: Synthetic peptide
KYAFF

SEQ ID NO: 52 - protein, 5 aa, synthetic construct
  REGION 1..5: note = Description of Artificial Sequence: Synthetic peptide
KYAFF

SEQ ID NO: 53 - protein, 5 aa, synthetic construct
  REGION 1..5: note = Description of Artificial Sequence: Synthetic peptide
KYAFF

SEQ ID NO: 54 - protein, 5 aa, synthetic construct
  REGION 1..5: note = Description of Artificial Sequence: Synthetic peptide
KYAFN

SEQ ID NO: 55 - protein, 5 aa, synthetic construct
  REGION 1..5: note = Description of Artificial Sequence: Synthetic peptide
KYAFN

SEQ ID NO: 56 - protein, 5 aa, synthetic construct
  REGION 1..5: note = Description of Artificial Sequence: Synthetic peptide
KYAFN

SEQ ID NO: 57 - protein, 5 aa, synthetic construct
  REGION 1..5: note = Description of Artificial Sequence: Synthetic peptide
KYAAF

SEQ ID NO: 58 - protein, 5 aa, synthetic construct
  REGION 1..5: note = Description of Artificial Sequence: Synthetic peptide
KYAAF

SEQ ID NO: 59 - protein, 5 aa, synthetic construct
  REGION 1..5: note = Description of Artificial Sequence: Synthetic peptide
KYAAF

SEQ ID NO: 60 - protein, 5 aa, synthetic construct
  REGION 1..5: note = Description of Artificial Sequence: Synthetic peptide
KYAAN

SEQ ID NO: 61 - protein, 5 aa, synthetic construct
  REGION 1..5: note = Description of Artificial Sequence: Synthetic peptide
KYAAN

SEQ ID NO: 62 - protein, 5 aa, synthetic construct
  REGION 1..5: note = Description of Artificial Sequence: Synthetic peptide
KYAAN

SEQ ID NO: 63 - protein, 5 aa, synthetic construct
  REGION 1..5: note = Description of Artificial Sequence: Synthetic peptide
KYYFF

SEQ ID NO: 64 - protein, 5 aa, synthetic construct
  REGION 1..5: note = Description of Artificial Sequence: Synthetic peptide
KYYFF

SEQ ID NO: 65 - protein, 5 aa, synthetic construct
  REGION 1..5: note = Description of Artificial Sequence: Synthetic peptide
KYYFF

SEQ ID NO: 66 - protein, 5 aa, synthetic construct
  REGION 1..5: note = Description of Artificial Sequence: Synthetic peptide
KYYFN

SEQ ID NO: 67 - protein, 5 aa, synthetic construct
  REGION 1..5: note = Description of Artificial Sequence: Synthetic peptide
KYYFN

SEQ ID NO: 68 - protein, 5 aa, synthetic construct
  REGION 1..5: note = Description of Artificial Sequence: Synthetic peptide
KYYFN

SEQ ID NO: 69 - protein, 5 aa, synthetic construct
  REGION 1..5: note = Description of Artificial Sequence: Synthetic peptide
KYYAF

SEQ ID NO: 70 - protein, 5 aa, synthetic construct
  REGION 1..5: note = Description of Artificial Sequence: Synthetic peptide
KYYAF

SEQ ID NO: 71 - protein, 5 aa, synthetic construct
  REGION 1..5: note = Description of Artificial Sequence: Synthetic peptide
KYYAF

SEQ ID NO: 72 - protein, 5 aa, synthetic construct
  REGION 1..5: note = Description of Artificial Sequence: Synthetic peptide
KYYAN

SEQ ID NO: 73 - protein, 5 aa, synthetic construct
  REGION 1..5: note = Description of Artificial Sequence: Synthetic peptide
KYYAN

SEQ ID NO: 74 - protein, 5 aa, synthetic construct
  REGION 1..5: note = Description of Artificial Sequence: Synthetic peptide
KYYAN

SEQ ID NO: 75 - protein, 5 aa, synthetic construct
  REGION 1..5: note = Description of Artificial Sequence: Synthetic peptide
RDAFF

SEQ ID NO: 76 - protein, 5 aa, synthetic construct
  REGION 1..5: note = Description of Artificial Sequence: Synthetic peptide
RDAFF

SEQ ID NO: 77 - protein, 5 aa, synthetic construct
  REGION 1..5: note = Description of Artificial Sequence: Synthetic peptide
RDAFF

SEQ ID NO: 78 - protein, 5 aa, synthetic construct
  REGION 1..5: note = Description of Artificial Sequence: Synthetic peptide
RDAFN

SEQ ID NO: 79 - protein, 5 aa, synthetic construct
  REGION 1..5: note = Description of Artificial Sequence: Synthetic peptide
RDAFN

SEQ ID NO: 80 - protein, 5 aa, synthetic construct
  REGION 1..5: note = Description of Artificial Sequence: Synthetic peptide
RDAFN

SEQ ID NO: 81 - protein, 5 aa, synthetic construct
  REGION 1..5: note = Description of Artificial Sequence: Synthetic peptide
RDAAF

SEQ ID NO: 82 - protein, 5 aa, synthetic construct
  REGION 1..5: note = Description of Artificial Sequence: Synthetic peptide
RDAAF

SEQ ID NO: 83 - protein, 5 aa, synthetic construct
  REGION 1..5: note = Description of Artificial Sequence: Synthetic peptide
RDAAF

SEQ ID NO: 84 - protein, 5 aa, synthetic construct
  REGION 1..5: note = Description of Artificial Sequence: Synthetic peptide
RDAAN

SEQ ID NO: 85 - protein, 5 aa, synthetic construct
  REGION 1..5: note = Description of Artificial Sequence: Synthetic peptide
RDAAN

SEQ ID NO: 86 - protein, 5 aa, synthetic construct
  REGION 1..5: note = Description of Artificial Sequence: Synthetic peptide
RDAAN

SEQ ID NO: 87 - protein, 5 aa, synthetic construct
  REGION 1..5: note = Description of Artificial Sequence: Synthetic peptide
RDYFF

SEQ ID NO: 88 - protein, 5 aa, synthetic construct
  REGION 1..5: note = Description of Artificial Sequence: Synthetic peptide
RDYFF

SEQ ID NO: 89 - protein, 5 aa, synthetic construct
  REGION 1..5: note = Description of Artificial Sequence: Synthetic peptide
RDYFF

SEQ ID NO: 90 - protein, 5 aa, synthetic construct
  REGION 1..5: note = Description of Artificial Sequence: Synthetic peptide
RDYFN

SEQ ID NO: 91 - protein, 5 aa, synthetic construct
  REGION 1..5: note = Description of Artificial Sequence: Synthetic peptide
RDYFN

SEQ ID NO: 92 - protein, 5 aa, synthetic construct
  REGION 1..5: note = Description of Artificial Sequence: Synthetic peptide
RDYFN

SEQ ID NO: 93 - protein, 5 aa, synthetic construct
  REGION 1..5: note = Description of Artificial Sequence: Synthetic peptide
RDYAF

SEQ ID NO: 94 - protein, 5 aa, synthetic construct
  REGION 1..5: note = Description of Artificial Sequence: Synthetic peptide
RDYAF

SEQ ID NO: 95 - protein, 5 aa, synthetic construct
  REGION 1..5: note = Description of Artificial Sequence: Synthetic peptide
RDYAF

SEQ ID NO: 96 - protein, 5 aa, synthetic construct
  REGION 1..5: note = Description of Artificial Sequence: Synthetic peptide
RDYAN

SEQ ID NO: 97 - protein, 5 aa, synthetic construct
  REGION 1..5: note = Description of Artificial Sequence: Synthetic peptide
RDYAN

SEQ ID NO: 98 - protein, 5 aa, synthetic construct
  REGION 1..5: note = Description of Artificial Sequence: Synthetic peptide
RDYAN

SEQ ID NO: 99 - protein, 5 aa, synthetic construct
  REGION 1..5: note = Description of Artificial Sequence: Synthetic peptide
RYAFF

SEQ ID NO: 100 - protein, 5 aa, synthetic construct
  REGION 1..5: note = Description of Artificial Sequence: Synthetic peptide
RYAFF

SEQ ID NO: 101 - protein, 5 aa, synthetic construct
  REGION 1..5: note = Description of Artificial Sequence: Synthetic peptide
RYAFF

SEQ ID NO: 102 - protein, 5 aa, synthetic construct
  REGION 1..5: note = Description of Artificial Sequence: Synthetic peptide
RYAFN

SEQ ID NO: 103 - protein, 5 aa, synthetic construct
  REGION 1..5: note = Description of Artificial Sequence: Synthetic peptide
RYAFN

SEQ ID NO: 104 - protein, 5 aa, synthetic construct
  REGION 1..5: note = Description of Artificial Sequence: Synthetic peptide
RYAFN

SEQ ID NO: 105 - protein, 5 aa, synthetic construct
  REGION 1..5: note = Description of Artificial Sequence: Synthetic peptide
RYAAF

SEQ ID NO: 106 - protein, 5 aa, synthetic construct
  REGION 1..5: note = Description of Artificial Sequence: Synthetic peptide
RYAAF

SEQ ID NO: 107 - protein, 5 aa, synthetic construct
  REGION 1..5: note = Description of Artificial Sequence: Synthetic peptide
RYAAF

SEQ ID NO: 108 - protein, 5 aa, synthetic construct
  REGION 1..5: note = Description of Artificial Sequence: Synthetic peptide
RYAAN

SEQ ID NO: 109 - protein, 5 aa, synthetic construct
  REGION 1..5: note = Description of Artificial Sequence: Synthetic peptide
RYAAN

SEQ ID NO: 110 - protein, 5 aa, synthetic construct
  REGION 1..5: note = Description of Artificial Sequence: Synthetic peptide
RYAAN

SEQ ID NO: 111 - protein, 5 aa, synthetic construct
  REGION 1..5: note = Description of Artificial Sequence: Synthetic peptide
RYYFF

SEQ ID NO: 112 - protein, 5 aa, synthetic construct
  REGION 1..5: note = Description of Artificial Sequence: Synthetic peptide
RYYFF

SEQ ID NO: 113 - protein, 5 aa, synthetic construct
  REGION 1..5: note = Description of Artificial Sequence: Synthetic peptide
RYYFF

SEQ ID NO: 114 - protein, 5 aa, synthetic construct
  REGION 1..5: note = Description of Artificial Sequence: Synthetic peptide
RYYFN

SEQ ID NO: 115 - protein, 5 aa, synthetic construct
  REGION 1..5: note = Description of Artificial Sequence: Synthetic peptide
RYYFN

SEQ ID NO: 116 - protein, 5 aa, synthetic construct
  REGION 1..5: note = Description of Artificial Sequence: Synthetic peptide
RYYFN

SEQ ID NO: 117 - protein, 5 aa, synthetic construct
  REGION 1..5: note = Description of Artificial Sequence: Synthetic peptide
RYYAF

SEQ ID NO: 118 - protein, 5 aa, synthetic construct
  REGION 1..5: note = Description of Artificial Sequence: Synthetic peptide
RYYAF

SEQ ID NO: 119 - protein, 5 aa, synthetic construct
  REGION 1..5: note = Description of Artificial Sequence: Synthetic peptide
RYYAF

SEQ ID NO: 120 - protein, 5 aa, synthetic construct
  REGION 1..5: note = Description of Artificial Sequence: Synthetic peptide
RYYAN

SEQ ID NO: 121 - protein, 5 aa, synthetic construct
  REGION 1..5: note = Description of Artificial Sequence: Synthetic peptide
RYYAN

SEQ ID NO: 122 - protein, 5 aa, synthetic construct
  REGION 1..5: note = Description of Artificial Sequence: Synthetic peptide
RYYAN

SEQ ID NO: 123 - protein, 5 aa, synthetic construct
  REGION 1..5: note = Description of Artificial Sequence: Synthetic peptide
CDAFF

SEQ ID NO: 124 - protein, 5 aa, synthetic construct
  REGION 1..5: note = Description of Artificial Sequence: Synthetic peptide
CDAFF

SEQ ID NO: 125 - protein, 5 aa, synthetic construct
  REGION 1..5: note = Description of Artificial Sequence: Synthetic peptide
CDAFF

SEQ ID NO: 126 - protein, 5 aa, synthetic construct
  REGION 1..5: note = Description of Artificial Sequence: Synthetic peptide
CDAFN

SEQ ID NO: 127 - protein, 5 aa, synthetic construct
  REGION 1..5: note = Description of Artificial Sequence: Synthetic peptide
CDAFN

SEQ ID NO: 128 - protein, 5 aa, synthetic construct
  REGION 1..5: note = Description of Artificial Sequence: Synthetic peptide
CDAFN

SEQ ID NO: 129 - protein, 5 aa, synthetic construct
  REGION 1..5: note = Description of Artificial Sequence: Synthetic peptide
CDAAF

SEQ ID NO: 130 - protein, 5 aa, synthetic construct
  REGION 1..5: note = Description of Artificial Sequence: Synthetic peptide
CDAAF

SEQ ID NO: 131 - protein, 5 aa, synthetic construct
  REGION 1..5: note = Description of Artificial Sequence: Synthetic peptide
CDAAF

SEQ ID NO: 132 - protein, 5 aa, synthetic construct
  REGION 1..5: note = Description of Artificial Sequence: Synthetic peptide
CDAAN

SEQ ID NO: 133 - protein, 5 aa, synthetic construct
  REGION 1..5: note = Description of Artificial Sequence: Synthetic peptide
CDAAN

SEQ ID NO: 134 - protein, 5 aa, synthetic construct
  REGION 1..5: note = Description of Artificial Sequence: Synthetic peptide
CDAAN

SEQ ID NO: 135 - protein, 5 aa, synthetic construct
  REGION 1..5: note = Description of Artificial Sequence: Synthetic peptide
CDYFF

SEQ ID NO: 136 - protein, 5 aa, synthetic construct
  REGION 1..5: note = Description of Artificial Sequence: Synthetic peptide
CDYFF

SEQ ID NO: 137 - protein, 5 aa, synthetic construct
  REGION 1..5: note = Description of Artificial Sequence: Synthetic peptide
CDYFF

SEQ ID NO: 138 - protein, 5 aa, synthetic construct
  REGION 1..5: note = Description of Artificial Sequence: Synthetic peptide
CDYFN

SEQ ID NO: 139 - protein, 5 aa, synthetic construct
  REGION 1..5: note = Description of Artificial Sequence: Synthetic peptide
CDYFN

SEQ ID NO: 140 - protein, 5 aa, synthetic construct
  REGION 1..5: note = Description of Artificial Sequence: Synthetic peptide
CDYFN

SEQ ID NO: 141 - protein, 5 aa, synthetic construct
  REGION 1..5: note = Description of Artificial Sequence: Synthetic peptide
CDYAF

SEQ ID NO: 142 - protein, 5 aa, synthetic construct
  REGION 1..5: note = Description of Artificial Sequence: Synthetic peptide
CDYAF

SEQ ID NO: 143 - protein, 5 aa, synthetic construct
  REGION 1..5: note = Description of Artificial Sequence: Synthetic peptide
CDYAF

SEQ ID NO: 144 - protein, 5 aa, synthetic construct
  REGION 1..5: note = Description of Artificial Sequence: Synthetic peptide
CDYAN

SEQ ID NO: 145 - protein, 5 aa, synthetic construct
  REGION 1..5: note = Description of Artificial Sequence: Synthetic peptide
CDYAN

SEQ ID NO: 146 - protein, 5 aa, synthetic construct
  REGION 1..5: note = Description of Artificial Sequence: Synthetic peptide
CDYAN

SEQ ID NO: 147 - protein, 5 aa, synthetic construct
  REGION 1..5: note = Description of Artificial Sequence: Synthetic peptide
CYAFF

SEQ ID NO: 148 - protein, 5 aa, synthetic construct
  REGION 1..5: note = Description of Artificial Sequence: Synthetic peptide
CYAFF

SEQ ID NO: 149 - protein, 5 aa, synthetic construct
  REGION 1..5: note = Description of Artificial Sequence: Synthetic peptide
CYAFF

SEQ ID NO: 150 - protein, 5 aa, synthetic construct
  REGION 1..5: note = Description of Artificial Sequence: Synthetic peptide
CYAFN

SEQ ID NO: 151 - protein, 5 aa, synthetic construct
  REGION 1..5: note = Description of Artificial Sequence: Synthetic peptide
CYAFN

SEQ ID NO: 152 - protein, 5 aa, synthetic construct
  REGION 1..5: note = Description of Artificial Sequence: Synthetic peptide
CYAFN

SEQ ID NO: 153 - protein, 5 aa, synthetic construct
  REGION 1..5: note = Description of Artificial Sequence: Synthetic peptide
CYAAF

SEQ ID NO: 154 - protein, 5 aa, synthetic construct
  REGION 1..5: note = Description of Artificial Sequence: Synthetic peptide
CYAAF

SEQ ID NO: 155 - protein, 5 aa, synthetic construct
  REGION 1..5: note = Description of Artificial Sequence: Synthetic peptide
CYAAF

SEQ ID NO: 156 - protein, 5 aa, synthetic construct
  REGION 1..5: note = Description of Artificial Sequence: Synthetic peptide
CYAAN

SEQ ID NO: 157 - protein, 5 aa, synthetic construct
  REGION 1..5: note = Description of Artificial Sequence: Synthetic peptide
CYAAN

SEQ ID NO: 158 - protein, 5 aa, synthetic construct
  REGION 1..5: note = Description of Artificial Sequence: Synthetic peptide
CYAAN

SEQ ID NO: 159 - protein, 5 aa, synthetic construct
  REGION 1..5: note = Description of Artificial Sequence: Synthetic peptide
CYYFF

SEQ ID NO: 160 - protein, 5 aa, synthetic construct
  REGION 1..5: note = Description of Artificial Sequence: Synthetic peptide
CYYFF

SEQ ID NO: 161 - protein, 5 aa, synthetic construct
  REGION 1..5: note = Description of Artificial Sequence: Synthetic peptide
CYYFF

SEQ ID NO: 162 - protein, 5 aa, synthetic construct
  REGION 1..5: note = Description of Artificial Sequence: Synthetic peptide
CYYFN

SEQ ID NO: 163 - protein, 5 aa, synthetic construct
  REGION 1..5: note = Description of Artificial Sequence: Synthetic peptide
CYYFN

SEQ ID NO: 164 - protein, 5 aa, synthetic construct
  REGION 1..5: note = Description of Artificial Sequence: Synthetic peptide
CYYFN

SEQ ID NO: 165 - protein, 5 aa, synthetic construct
  REGION 1..5: note = Description of Artificial Sequence: Synthetic peptide
CYYAF

SEQ ID NO: 166 - protein, 5 aa, synthetic construct
  REGION 1..5: note = Description of Artificial Sequence: Synthetic peptide
CYYAF

SEQ ID NO: 167 - protein, 5 aa, synthetic construct
  REGION 1..5: note = Description of Artificial Sequence: Synthetic peptide
CYYAF

SEQ ID NO: 168 - protein, 5 aa, synthetic construct
  REGION 1..5: note = Description of Artificial Sequence: Synthetic peptide
CYYAN

SEQ ID NO: 169 - protein, 5 aa, synthetic construct
  REGION 1..5: note = Description of Artificial Sequence: Synthetic peptide
CYYAN

SEQ ID NO: 170 - protein, 5 aa, synthetic construct
  REGION 1..5: note = Description of Artificial Sequence: Synthetic peptide
CYYAN

SEQ ID NO: 171 - protein, 5 aa, synthetic construct
  REGION 1..5: note = Description of Artificial Sequence: Synthetic peptide
SDAFF

SEQ ID NO: 172 - protein, 5 aa, synthetic construct
  REGION 1..5: note = Description of Artificial Sequence: Synthetic peptide
SDAFF

SEQ ID NO: 173 - protein, 5 aa, synthetic construct
  REGION 1..5: note = Description of Artificial Sequence: Synthetic peptide
SDAFF

SEQ ID NO: 174 - protein, 5 aa, synthetic construct
  REGION 1..5: note = Description of Artificial Sequence: Synthetic peptide
SDAFN

SEQ ID NO: 175 - protein, 5 aa, synthetic construct
  REGION 1..5: note = Description of Artificial Sequence: Synthetic peptide
SDAFN

SEQ ID NO: 176 - protein, 5 aa, synthetic construct
  REGION 1..5: note = Description of Artificial Sequence: Synthetic peptide
SDAFN

SEQ ID NO: 177 - protein, 5 aa, synthetic construct
  REGION 1..5: note = Description of Artificial Sequence: Synthetic peptide
SDAAF

SEQ ID NO: 178 - protein, 5 aa, synthetic construct
  REGION 1..5: note = Description of Artificial Sequence: Synthetic peptide
SDAAF

SEQ ID NO: 179 - protein, 5 aa, synthetic construct
  REGION 1..5: note = Description of Artificial Sequence: Synthetic peptide
SDAAF

SEQ ID NO: 180 - protein, 5 aa, synthetic construct
  REGION 1..5: note = Description of Artificial Sequence: Synthetic peptide
SDAAN

SEQ ID NO: 181 - protein, 5 aa, synthetic construct
  REGION 1..5: note = Description of Artificial Sequence: Synthetic peptide
SDAAN

SEQ ID NO: 182 - protein, 5 aa, synthetic construct
  REGION 1..5: note = Description of Artificial Sequence: Synthetic peptide
SDAAN

SEQ ID NO: 183 - protein, 5 aa, synthetic construct
  REGION 1..5: note = Description of Artificial Sequence: Synthetic peptide
SDYFF

SEQ ID NO: 184 - protein, 5 aa, synthetic construct
  REGION 1..5: note = Description of Artificial Sequence: Synthetic peptide
SDYFF

SEQ ID NO: 185 - protein, 5 aa, synthetic construct
  REGION 1..5: note = Description of Artificial Sequence: Synthetic peptide
SDYFF

SEQ ID NO: 186 - protein, 5 aa, synthetic construct
  REGION 1..5: note = Description of Artificial Sequence: Synthetic peptide
SDYFN

SEQ ID NO: 187 - protein, 5 aa, synthetic construct
  REGION 1..5: note = Description of Artificial Sequence: Synthetic peptide
SDYFN

SEQ ID NO: 188 - protein, 5 aa, synthetic construct
  REGION 1..5: note = Description of Artificial Sequence: Synthetic peptide
SDYFN

SEQ ID NO: 189 - protein, 5 aa, synthetic construct
  REGION 1..5: note = Description of Artificial Sequence: Synthetic peptide
SDYAF

SEQ ID NO: 190 - protein, 5 aa, synthetic construct
  REGION 1..5: note = Description of Artificial Sequence: Synthetic peptide
SDYAF

SEQ ID NO: 191 - protein, 5 aa, synthetic construct
  REGION 1..5: note = Description of Artificial Sequence: Synthetic peptide
SDYAF

SEQ ID NO: 192 - protein, 5 aa, synthetic construct
  REGION 1..5: note = Description of Artificial Sequence: Synthetic peptide
SDYAN

SEQ ID NO: 193 - protein, 5 aa, synthetic construct
  REGION 1..5: note = Description of Artificial Sequence: Synthetic peptide
SDYAN

SEQ ID NO: 194 - protein, 5 aa, synthetic construct
  REGION 1..5: note = Description of Artificial Sequence: Synthetic peptide
SDYAN

SEQ ID NO: 195 - protein, 5 aa, synthetic construct
  REGION 1..5: note = Description of Artificial Sequence: Synthetic peptide
SYAFF

SEQ ID NO: 196 - protein, 5 aa, synthetic construct
  REGION 1..5: note = Description of Artificial Sequence: Synthetic peptide
SYAFF

SEQ ID NO: 197 - protein, 5 aa, synthetic construct
  REGION 1..5: note = Description of Artificial Sequence: Synthetic peptide
SYAFF

SEQ ID NO: 198 - protein, 5 aa, synthetic construct
  REGION 1..5: note = Description of Artificial Sequence: Synthetic peptide
SYAFN

SEQ ID NO: 199 - protein, 5 aa, synthetic construct
  REGION 1..5: note = Description of Artificial Sequence: Synthetic peptide
SYAFN

SEQ ID NO: 200 - protein, 5 aa, synthetic construct
  REGION 1..5: note = Description of Artificial Sequence: Synthetic peptide
SYAFN

SEQ ID NO: 201 - protein, 5 aa, synthetic construct
  REGION 1..5: note = Description of Artificial Sequence: Synthetic peptide
SYAAF

SEQ ID NO: 202 - protein, 5 aa, synthetic construct
  REGION 1..5: note = Description of Artificial Sequence: Synthetic peptide
SYAAF

SEQ ID NO: 203 - protein, 5 aa, synthetic construct
  REGION 1..5: note = Description of Artificial Sequence: Synthetic peptide
SYAAF

SEQ ID NO: 204 - protein, 5 aa, synthetic construct
  REGION 1..5: note = Description of Artificial Sequence: Synthetic peptide
SYAAN

SEQ ID NO: 205 - protein, 5 aa, synthetic construct
  REGION 1..5: note = Description of Artificial Sequence: Synthetic peptide
SYAAN

SEQ ID NO: 206 - protein, 5 aa, synthetic construct
  REGION 1..5: note = Description of Artificial Sequence: Synthetic peptide
SYAAN

SEQ ID NO: 207 - protein, 5 aa, synthetic construct
  REGION 1..5: note = Description of Artificial Sequence: Synthetic peptide
SYYFF

SEQ ID NO: 208 - protein, 5 aa, synthetic construct
  REGION 1..5: note = Description of Artificial Sequence: Synthetic peptide
SYYFF

SEQ ID NO: 209 - protein, 5 aa, synthetic construct
  REGION 1..5: note = Description of Artificial Sequence: Synthetic peptide
SYYFF

SEQ ID NO: 210 - protein, 5 aa, synthetic construct
  REGION 1..5: note = Description of Artificial Sequence: Synthetic peptide
SYYFN

SEQ ID NO: 211 - protein, 5 aa, synthetic construct
  REGION 1..5: note = Description of Artificial Sequence: Synthetic peptide
SYYFN

SEQ ID NO: 212 - protein, 5 aa, synthetic construct
  REGION 1..5: note = Description of Artificial Sequence: Synthetic peptide
SYYFN

SEQ ID NO: 213 - protein, 5 aa, synthetic construct
  REGION 1..5: note = Description of Artificial Sequence: Synthetic peptide
SYYAF

SEQ ID NO: 214 - protein, 5 aa, synthetic construct
  REGION 1..5: note = Description of Artificial Sequence: Synthetic peptide
SYYAF

SEQ ID NO: 215 - protein, 5 aa, synthetic construct
  REGION 1..5: note = Description of Artificial Sequence: Synthetic peptide
SYYAF

SEQ ID NO: 216 - protein, 5 aa, synthetic construct
  REGION 1..5: note = Description of Artificial Sequence: Synthetic peptide
SYYAN

SEQ ID NO: 217 - protein, 5 aa, synthetic construct
  REGION 1..5: note = Description of Artificial Sequence: Synthetic peptide
SYYAN

SEQ ID NO: 218 - protein, 5 aa, synthetic construct
  REGION 1..5: note = Description of Artificial Sequence: Synthetic peptide
SYYAN

SEQ ID NO: 219 - protein, 5 aa, synthetic construct
  REGION 1..5: note = Description of Artificial Sequence: Synthetic peptide
TDAFF

SEQ ID NO: 220 - protein, 5 aa, synthetic construct
  REGION 1..5: note = Description of Artificial Sequence: Synthetic peptide
TDAFF

SEQ ID NO: 221 - protein, 5 aa, synthetic construct
  REGION 1..5: note = Description of Artificial Sequence: Synthetic peptide
TDAFF

SEQ ID NO: 222 - protein, 5 aa, synthetic construct
  REGION 1..5: note = Description of Artificial Sequence: Synthetic peptide
TDAFN

SEQ ID NO: 223 - protein, 5 aa, synthetic construct
  REGION 1..5: note = Description of Artificial Sequence: Synthetic peptide
TDAFN

SEQ ID NO: 224 - protein, 5 aa, synthetic construct
  REGION 1..5: note = Description of Artificial Sequence: Synthetic peptide
TDAFN

SEQ ID NO: 225 - protein, 5 aa, synthetic construct
  REGION 1..5: note = Description of Artificial Sequence: Synthetic peptide
TDAAF

SEQ ID NO: 226 - protein, 5 aa, synthetic construct
  REGION 1..5: note = Description of Artificial Sequence: Synthetic peptide
TDAAF

SEQ ID NO: 227 - protein, 5 aa, synthetic construct
  REGION 1..5: note = Description of Artificial Sequence: Synthetic peptide
TDAAF

SEQ ID NO: 228 - protein, 5 aa, synthetic construct
  REGION 1..5: note = Description of Artificial Sequence: Synthetic peptide
TDAAN

SEQ ID NO: 229 - protein, 5 aa, synthetic construct
  REGION 1..5: note = Description of Artificial Sequence: Synthetic peptide
TDAAN

SEQ ID NO: 230 - protein, 5 aa, synthetic construct
  REGION 1..5: note = Description of Artificial Sequence: Synthetic peptide
TDAAN

SEQ ID NO: 231 - protein, 5 aa, synthetic construct
  REGION 1..5: note = Description of Artificial Sequence: Synthetic peptide
TDYFF

SEQ ID NO: 232 - protein, 5 aa, synthetic construct
  REGION 1..5: note = Description of Artificial Sequence: Synthetic peptide
TDYFF

SEQ ID NO: 233 - protein, 5 aa, synthetic construct
  REGION 1..5: note = Description of Artificial Sequence: Synthetic peptide
TDYFF

SEQ ID NO: 234 - protein, 5 aa, synthetic construct
  REGION 1..5: note = Description of Artificial Sequence: Synthetic peptide
TDYFN

SEQ ID NO: 235 - protein, 5 aa, synthetic construct
  REGION 1..5: note = Description of Artificial Sequence: Synthetic peptide
TDYFN

SEQ ID NO: 236 - protein, 5 aa, synthetic construct
  REGION 1..5: note = Description of Artificial Sequence: Synthetic peptide
TDYFN

SEQ ID NO: 237 - protein, 5 aa, synthetic construct
  REGION 1..5: note = Description of Artificial Sequence: Synthetic peptide
TDYAF

SEQ ID NO: 238 - protein, 5 aa, synthetic construct
  REGION 1..5: note = Description of Artificial Sequence: Synthetic peptide
TDYAF

SEQ ID NO: 239 - protein, 5 aa, synthetic construct
  REGION 1..5: note = Description of Artificial Sequence: Synthetic peptide
TDYAF

SEQ ID NO: 240 - protein, 5 aa, synthetic construct
  REGION 1..5: note = Description of Artificial Sequence: Synthetic peptide
TDYAN

SEQ ID NO: 241 - protein, 5 aa, synthetic construct
  REGION 1..5: note = Description of Artificial Sequence: Synthetic peptide
TDYAN

SEQ ID NO: 242 - protein, 5 aa, synthetic construct
  REGION 1..5: note = Description of Artificial Sequence: Synthetic peptide
TDYAN

SEQ ID NO: 243 - protein, 5 aa, synthetic construct
  REGION 1..5: note = Description of Artificial Sequence: Synthetic peptide
TYAFF

SEQ ID NO: 244 - protein, 5 aa, synthetic construct
  REGION 1..5: note = Description of Artificial Sequence: Synthetic peptide
TYAFF

SEQ ID NO: 245 - protein, 5 aa, synthetic construct
  REGION 1..5: note = Description of Artificial Sequence: Synthetic peptide
TYAFF

SEQ ID NO: 246 - protein, 5 aa, synthetic construct
  REGION 1..5: note = Description of Artificial Sequence: Synthetic peptide
TYAFN

SEQ ID NO: 247 - protein, 5 aa, synthetic construct
  REGION 1..5: note = Description of Artificial Sequence: Synthetic peptide
TYAFN

SEQ ID NO: 248 - protein, 5 aa, synthetic construct
  REGION 1..5: note = Description of Artificial Sequence: Synthetic peptide
TYAFN

SEQ ID NO: 249 - protein, 5 aa, synthetic construct
  REGION 1..5: note = Description of Artificial Sequence: Synthetic peptide
TYAAF

SEQ ID NO: 250 - protein, 5 aa, synthetic construct
  REGION 1..5: note = Description of Artificial Sequence: Synthetic peptide
TYAAF

SEQ ID NO: 251 - protein, 5 aa, synthetic construct
  REGION 1..5: note = Description of Artificial Sequence: Synthetic peptide
TYAAF

SEQ ID NO: 252 - protein, 5 aa, synthetic construct
  REGION 1..5: note = Description of Artificial Sequence: Synthetic peptide
TYAAN

SEQ ID NO: 253 - protein, 5 aa, synthetic construct
  REGION 1..5: note = Description of Artificial Sequence: Synthetic peptide
TYAAN

SEQ ID NO: 254 - protein, 5 aa, synthetic construct
  REGION 1..5: note = Description of Artificial Sequence: Synthetic peptide
TYAAN

SEQ ID NO: 255 - protein, 5 aa, synthetic construct
  REGION 1..5: note = Description of Artificial Sequence: Synthetic peptide
TYYFF

SEQ ID NO: 256 - protein, 5 aa, synthetic construct
  REGION 1..5: note = Description of Artificial Sequence: Synthetic peptide
TYYFF

SEQ ID NO: 257 - protein, 5 aa, synthetic construct
  REGION 1..5: note = Description of Artificial Sequence: Synthetic peptide
TYYFF

SEQ ID NO: 258 - protein, 5 aa, synthetic construct
  REGION 1..5: note = Description of Artificial Sequence: Synthetic peptide
TYYFN

SEQ ID NO: 259 - protein, 5 aa, synthetic construct
  REGION 1..5: note = Description of Artificial Sequence: Synthetic peptide
TYYFN

SEQ ID NO: 260 - protein, 5 aa, synthetic construct
  REGION 1..5: note = Description of Artificial Sequence: Synthetic peptide
TYYFN

SEQ ID NO: 261 - protein, 5 aa, synthetic construct
  REGION 1..5: note = Description of Artificial Sequence: Synthetic peptide
TYYAF

SEQ ID NO: 262 - protein, 5 aa, synthetic construct
  REGION 1..5: note = Description of Artificial Sequence: Synthetic peptide
TYYAF

SEQ ID NO: 263 - protein, 5 aa, synthetic construct
  REGION 1..5: note = Description of Artificial Sequence: Synthetic peptide
TYYAF

SEQ ID NO: 264 - protein, 5 aa, synthetic construct
  REGION 1..5: note = Description of Artificial Sequence: Synthetic peptide
TYYAN

SEQ ID NO: 265 - protein, 5 aa, synthetic construct
  REGION 1..5: note = Description of Artificial Sequence: Synthetic peptide
TYYAN

SEQ ID NO: 266 - protein, 5 aa, synthetic construct
  REGION 1..5: note = Description of Artificial Sequence: Synthetic peptide
TYYAN

SEQ ID NO: 267 - protein, 5 aa, synthetic construct
  REGION 1..5: note = Description of Artificial Sequence: Synthetic peptide
LDAFF

SEQ ID NO: 268 - protein, 5 aa, synthetic construct
  REGION 1..5: note = Description of Artificial Sequence: Synthetic peptide
LDAFF

SEQ ID NO: 269 - protein, 5 aa, synthetic construct
  REGION 1..5: note = Description of Artificial Sequence: Synthetic peptide
LDAFF

SEQ ID NO: 270 - protein, 5 aa, synthetic construct
  REGION 1..5: note = Description of Artificial Sequence: Synthetic peptide
LDAFN

SEQ ID NO: 271 - protein, 5 aa, synthetic construct
  REGION 1..5: note = Description of Artificial Sequence: Synthetic peptide
LDAFN

SEQ ID NO: 272 - protein, 5 aa, synthetic construct
  REGION 1..5: note = Description of Artificial Sequence: Synthetic peptide
LDAFN

SEQ ID NO: 273 - protein, 5 aa, synthetic construct
  REGION 1..5: note = Description of Artificial Sequence: Synthetic peptide
LDAAF

SEQ ID NO: 274 - protein, 5 aa, synthetic construct
  REGION 1..5: note = Description of Artificial Sequence: Synthetic peptide
LDAAF

SEQ ID NO: 275 - protein, 5 aa, synthetic construct
  REGION 1..5: note = Description of Artificial Sequence: Synthetic peptide
LDAAF

SEQ ID NO: 276 - protein, 5 aa, synthetic construct
  REGION 1..5: note = Description of Artificial Sequence: Synthetic peptide
LDAAN

SEQ ID NO: 277 - protein, 5 aa, synthetic construct
  REGION 1..5: note = Description of Artificial Sequence: Synthetic peptide
LDAAN

SEQ ID NO: 278 - protein, 5 aa, synthetic construct
  REGION 1..5: note = Description of Artificial Sequence: Synthetic peptide
LDAAN

SEQ ID NO: 279 - protein, 5 aa, synthetic construct
  REGION 1..5: note = Description of Artificial Sequence: Synthetic peptide
LDYFF

SEQ ID NO: 280 - protein, 5 aa, synthetic construct
  REGION 1..5: note = Description of Artificial Sequence: Synthetic peptide
LDYFF

SEQ ID NO: 281 - protein, 5 aa, synthetic construct
  REGION 1..5: note = Description of Artificial Sequence: Synthetic peptide
LDYFF

SEQ ID NO: 282 - protein, 5 aa, synthetic construct
  REGION 1..5: note = Description of Artificial Sequence: Synthetic peptide
LDYFN

SEQ ID NO: 283 - protein, 5 aa, synthetic construct
  REGION 1..5: note = Description of Artificial Sequence: Synthetic peptide
LDYFN

SEQ ID NO: 284 - protein, 5 aa, synthetic construct
  REGION 1..5: note = Description of Artificial Sequence: Synthetic peptide
LDYFN

SEQ ID NO: 285 - protein, 5 aa, synthetic construct
  REGION 1..5: note = Description of Artificial Sequence: Synthetic peptide
LDYAF

SEQ ID NO: 286 - protein, 5 aa, synthetic construct
  REGION 1..5: note = Description of Artificial Sequence: Synthetic peptide
LDYAF

SEQ ID NO: 287 - protein, 5 aa, synthetic construct
  REGION 1..5: note = Description of Artificial Sequence: Synthetic peptide
LDYAF

SEQ ID NO: 288 - protein, 5 aa, synthetic construct
  REGION 1..5: note = Description of Artificial Sequence: Synthetic peptide
LDYAN

SEQ ID NO: 289 - protein, 5 aa, synthetic construct
  REGION 1..5: note = Description of Artificial Sequence: Synthetic peptide
LDYAN

SEQ ID NO: 290 - protein, 5 aa, synthetic construct
  REGION 1..5: note = Description of Artificial Sequence: Synthetic peptide
LDYAN

SEQ ID NO: 291 - protein, 5 aa, synthetic construct
  REGION 1..5: note = Description of Artificial Sequence: Synthetic peptide
LYAFF

SEQ ID NO: 292 - protein, 5 aa, synthetic construct
  REGION 1..5: note = Description of Artificial Sequence: Synthetic peptide
LYAFF

SEQ ID NO: 293 - protein, 5 aa, synthetic construct
  REGION 1..5: note = Description of Artificial Sequence: Synthetic peptide
LYAFF

SEQ ID NO: 294 - protein, 5 aa, synthetic construct
  REGION 1..5: note = Description of Artificial Sequence: Synthetic peptide
LYAFN

SEQ ID NO: 295 - protein, 5 aa, synthetic construct
  REGION 1..5: note = Description of Artificial Sequence: Synthetic peptide
LYAFN

SEQ ID NO: 296 - protein, 5 aa, synthetic construct
  REGION 1..5: note = Description of Artificial Sequence: Synthetic peptide
LYAFN

SEQ ID NO: 297 - protein, 5 aa, synthetic construct
  REGION 1..5: note = Description of Artificial Sequence: Synthetic peptide
LYAAF

SEQ ID NO: 298 - protein, 5 aa, synthetic construct
  REGION 1..5: note = Description of Artificial Sequence: Synthetic peptide
LYAAF

SEQ ID NO: 299 - protein, 5 aa, synthetic construct
  REGION 1..5: note = Description of Artificial Sequence: Synthetic peptide
LYAAF

SEQ ID NO: 300 - protein, 5 aa, synthetic construct
  REGION 1..5: note = Description of Artificial Sequence: Synthetic peptide
LYAAN

SEQ ID NO: 301 - protein, 5 aa, synthetic construct
  REGION 1..5: note = Description of Artificial Sequence: Synthetic peptide
LYAAN

SEQ ID NO: 302 - protein, 5 aa, synthetic construct
  REGION 1..5: note = Description of Artificial Sequence: Synthetic peptide
LYAAN

SEQ ID NO: 303 - protein, 5 aa, synthetic construct
  REGION 1..5: note = Description of Artificial Sequence: Synthetic peptide
LYYFF

SEQ ID NO: 304 - protein, 5 aa, synthetic construct
  REGION 1..5: note = Description of Artificial Sequence: Synthetic peptide
LYYFF

SEQ ID NO: 305 - protein, 5 aa, synthetic construct
  REGION 1..5: note = Description of Artificial Sequence: Synthetic peptide
LYYFF

SEQ ID NO: 306 - protein, 5 aa, synthetic construct
  REGION 1..5: note = Description of Artificial Sequence: Synthetic peptide
LYYFN

SEQ ID NO: 307 - protein, 5 aa, synthetic construct
  REGION 1..5: note = Description of Artificial Sequence: Synthetic peptide
LYYFN

SEQ ID NO: 308 - protein, 5 aa, synthetic construct
  REGION 1..5: note = Description of Artificial Sequence: Synthetic peptide
LYYFN

SEQ ID NO: 309 - protein, 5 aa, synthetic construct
  REGION 1..5: note = Description of Artificial Sequence: Synthetic peptide
LYYAF

SEQ ID NO: 310 - protein, 5 aa, synthetic construct
  REGION 1..5: note = Description of Artificial Sequence: Synthetic peptide
LYYAF

SEQ ID NO: 311 - protein, 5 aa, synthetic construct
  REGION 1..5: note = Description of Artificial Sequence: Synthetic peptide
LYYAF

SEQ ID NO: 312 - protein, 5 aa, synthetic construct
  REGION 1..5: note = Description of Artificial Sequence: Synthetic peptide
LYYAN

SEQ ID NO: 313 - protein, 5 aa, synthetic construct
  REGION 1..5: note = Description of Artificial Sequence: Synthetic peptide
LYYAN

SEQ ID NO: 314 - protein, 5 aa, synthetic construct
  REGION 1..5: note = Description of Artificial Sequence: Synthetic peptide
LYYAN

SEQ ID NO: 315 - protein, 6 aa, synthetic construct
  REGION 1..6: note = Description of Artificial Sequence: Synthetic 6xHis tag
HHHHHH

SEQ ID NO: 316 - protein, 671 aa, Moloney murine leukemia virus
LNIEDEHRLHETSKEPDVSLGSTWLSDFPQAWAETGGMGLAVRQAPLIIPLKATSTPVSIKQYPMSQEARLGIKPHIQRLLDQGILVPCQSPWNTPLLPVKKPGTNDYRPVQDLREVNKRVEDIHPTVPNPYNLLSGLPPSHQWYTVLDLKDAFFCLRLHPTSQPLFAFEWRDPEMGISGQLTWTRLPQGFKNSPTLFDEALHRDLADFRIQHPDLILLQYVDDLLLAATSELDCQQGTRALLQTLGNLGYRASAKKAQICQKQVKYLGYLLKEGQRWLTEARKETVMGQPTPKTPRQLREFLGTAGFCRLWIPGFAEMAAPLYPLTKTGTLFNWGPDQQKAYQEIKQALLTAPALGLPDLTKPFELFVDEKQGYAKGVLTQKLGPWRRPVAYLSKKLDPVAAGWPPCLRMVAAIAVLTKDAGKLTMGQPLVILAPHAVEALVKQPPDRWLSNARMTHYQALLLDTDRVQFGPVVALNPATLLPLPEEGLQHNCLDILAEAHGTRPDLTDQPLPDADHTWYTDGSSLLQEGQRKAGAAVTTETEVIWAKALPAGTSAQRAELIALTQALKMAEGKKLNVYTDSRYAFATAHIHGEIYRRRGLLTSEGKEIKNKDEILALLKALFLPKRLSIIHCPGHQKGHSAEARGNRMADQAARKAAITETPDTSTLLI

SEQ ID NO: 317 - protein, 671 aa, Moloney murine leukemia virus; identical to SEQ ID NO: 316